(19) United States
(12) Patent Application Publication
(10) Pub. No.: US 2026/0021066 A1
(43) Pub. Date: Jan. 22, 2026

(54) Chelation Therapy to Limit Cell Senescence

(71) Applicant: CLEMSON UNIVERSITY, CLEMSON, SC (US)

(72) Inventors: Narendra R Vyavahare, CLEMSON, SC (US); Shivani Arora, CLEMSON, SC (US)

(21) Appl. No.: 19/273,800

(22) Filed: Jul. 18, 2025

Related U.S. Application Data
(60) Provisional application No. 63/673,447, filed on Jul. 19, 2024.
(60) Provisional application No. 63/784,472, filed on Apr. 7, 2025.

(51) Int. Cl. A61K 31/198; A61K 9/51; A61P 13/12

(52) U.S. Cl. CPC A61K 31/198; A61K 9/5169; A61P 13/12

(57) ABSTRACT

The present application is generally directed to methods for chelating calcium deposits within calcified blood vessels, as well as methods and compositions for use in regulating various senescence-related inflammatory pathways.

### CROSS REFERENCE TO RELATED APPLICATION

This application claims priority to U.S. Provisional Application No. 63/673,447 and U.S. Provisional Application No. 63/784,472 having a filing date of Jul. 19, 2024 and Apr. 7, 2025, respectively, both of which are incorporated herein.

### FEDERAL RESEARCH STATEMENT

This invention was made with Government support under Contract No. HL145064, awarded by the National Institute of Health. The Government has certain rights in the invention.

### SEQUENCE LISTING

The instant application contains a Sequence Listing which has been submitted electronically in XML file format and is hereby incorporated by reference in its entirety. Said XML copy, created on Jul. 17, 2025, is named CXU-1080_SL.xml and is 54,803 bytes in size.

### BACKGROUND

When cells no longer divide and reproduce, but do not undergo apoptosis, they are said to be senescent. Aging causes a slow and gradual buildup of senescent cells in vital organs, specifically in the kidney, heart, aorta (blood vessels), muscles, lungs, brain, liver, bones, and even in an immune cell population. Senescent cells are important for a variety of functions within the body, such as wound healing, tumor suppression and embryonic regulation.

However, senescent cells additionally secrete a variety of pro-inflammatory cytokines, growth factors and proteases, collectively known as senescence associated secretory phenotype (SASP). One effect of SASP is to cause other, non-senescent cells to enter senescence, thereby causing a cascade. Additionally, senescent cells are highly correlated to age related diseases, including arthritis, atherosclerosis and neurodegenerative disorders. Hyperphosphatemia associated with chronic kidney disorder and aging-associated decline in kidney function is specifically responsible for the premature and accelerated accumulation of senescent cells in the blood vessels.

Senolytics have been previously described as a measure to reduce the number of senescent cells in a tissue. These senolytics typically function by causing a senescent cell to undergo apoptosis, i.e. cell death. However, this is not always favorable. For instance, in blood vessels mass apoptosis may lead to hemorrhage, increased strain on blood filtration systems and off-targeting associated side effects. What is needed in the art, therefore, is a therapy which may reduce the negative effects of senescent cells, without inducing said cells to undergo apoptosis.

### BRIEF DESCRIPTION OF THE FIGURES

FIGS. 1A-1D show the calcification of arterial vessels under alizarin red stain and MicroCT. FIG. 1C shows that early-stage and late-stage kidneys were enlarged in size, and FIG. 1D shows that creatinine was increased for early-stage and late-stage samples;

FIGS. 2A-2D are a series of graphs and charts showing the increase in SA-βGal, p21 and p19 expression;

FIG. 3A is a series of images showing aorta samples when subject to a high phosphate environment;

FIG. 3B-C is a series of graphs showing the increase of NLRP3, SA-βGal, Caspase3, IL-1β and IL-6 expression when arterial cells are subject to a high phosphate environment;

FIGS. 4A-4B is a series of images and graphs showing the relatively higher expression of Pit-1 in a high phosphate environment as compared to the high phosphate+EDTA aorta;

FIG. 5A is a series of images showing calcification by alizarin red staining;

FIG. 5B is a series of images showing calcification by MicroCT;

FIGS. 5C and 5D is a transcript level analysis of ossification markers OCN and RUNX2;

FIG. 5E shows the survival curve for control cells, cells treated with blank nanoparticles and cells treated with EDTA-loaded nanoparticles;

FIGS. 5F and 5G are graphs showing the concentration in serum of IL-1β and IL-6;

FIGS. 6A-6C are a series of graphs and images showing the transcriptional expression and activity of senescence and SASP markers;

FIGS. 7A-8B are a series of stains and summary data relating to the expression of Caspase3 and NLRP3 in cells after treatment with EDTA and ABT 263;

FIGS. 9A-9E are graphs and stains showing that treatment with EDTA and EDTA nanoparticles may decrease NLRP3, Capase3, IL-1β and IL-6;

FIG. 10 is a Venn diagram of protein isolated from the abdominal aorta of EDTA NP treated vs blank NP treated animals;

FIG. 11 is a plot showing the log p-value as a function of log2 fold change for the proteins differentially expressed; and

FIGS. 12A-12B are pie charts showing the top 25 and top 20 proteins upregulated and downregulated respectively after treatment with EDTA.

### SUMMARY

Generally, the present disclosure is directed to therapeutics for reducing the accumulation and effect of senescent cells within tissues, and the administration of said therapeutics.

For instance, the present claims are generally directed to a method for reducing senescent cell accumulation, the method comprising delivering a chelating agent to a tissue. Further, in other embodiments of the present disclosure, a method for treating a patient with vascular calcification, the method comprising administering a nanoparticle which comprises a polymeric component, a chelating agent and an antibody to the patient is described. In other embodiments, a method for reducing SASP in non-calcified tissue comprising administering to a patient a nanoparticle which comprises a polymeric component, such as a liposome, a chelating agent and an antibody to the patient may be described.

### DETAILED DESCRIPTION

Reference will now be made in detail to various embodiments of the disclosed subject matter, one or more examples of which are set forth below. Each embodiment is provided by way of explanation the subject matter, not limitation thereof. In fact, it will be apparent to those skilled in the art that various modifications and variations may be made in the present disclosure without departing from the scope or spirit of the subject matter. For instance, features illustrated or described as part of one embodiment, may be used in another embodiment to yield a still further embodiment.

Additionally, for the purposes of this disclosure, the terms “therapeutic agent”, “therapeutic”, “active agent”, “biologically active agent”, “biologically active ingredient”, or other common variations thereof are understood to be interchangeable.

In general, the present disclosure is directed to therapeutics which may be administered to a patient to reduce the accumulation of senescent cells, and the effects thereof. Further, the present application is additionally directed to method of administering said therapeutics. The therapeutics generally comprise a chelating agent. In some embodiments, a nanoparticle may comprise the chelating agent. In further embodiments, the nanoparticle may comprise peptides or proteins which exhibit tissue selectivity. The preceding statements serve only as a brief description of the therapeutic, and not as a limitation as to the makeup of a therapeutic, nor a method of administration.

Of susceptibility to calcification are blood vessels. A common age-related disease is atherosclerotic calcification, named as intimal calcification and elastin-specific medial arterial calcification, named as Monckeberg's arterioscelrosis. Healthy blood vessels typically experience vasoconstriction and vasodilation to raise or lower blood pressure, typically in reaction to some form of stimuli. However, in blood vessels which have become calcified, vasoconstriction and vasodilation are inhibited, leading to poor health outcomes.

While calcification has impacts on the elasticity and health of blood vessels, excess calcium accumulation can have negative effects elsewhere, and the present disclosure is not particularly limited to treating the calcification of blood vessels.

The inventors of the present disclosure have found that one surprising effect of increased calcium deposits throughout the body is increased expression of the NLRP3 inflammasome. While not being bound by theory, it is believed that excess extra-cellular calcium can stimulate the NLRP3 pathway, leading to increased inflammasome expression. Increased expression of the inflammasome is found to increase the likelihood that proximal cells become senescent as well, thereby increasing the total expression of senescent cells. In this sense, it may be said that extracellular calcium deposits may lead to a senescence cascade.

Additionally, the present inventors have found that extracellular calcium may increase the expression of SASP (senescence-associated secretory phenotype).

Further, the inventors of the present disclosure have found that in vascular calcification, the presence of senescent cells precedes calcification.

As stated above, however, senescent cells are not without their use. Senescent cells serve important functions, such as in wound healing and tumor prevention. Additionally, their elimination has been found to increase liver failure and cardiac fibrosis, among other things.

Accordingly, the present disclosure is generally directed to compositions and methods for reducing the accumulation of extracellular calcium deposits, as well as decreasing the expression of the NLRP3 inflammasome, as well as SASP. As will be made clear by the following disclosure, the present invention is not particularly limited to any one of these specific methods. For instance, the present inventors have found that the NLRP3 inflammasome and SASP expression can be reduced even in living tissues wherein extracellular calcium deposits are not significant.

The present inventors have found that an effective therapeutic for reducing extra-cellular calcium is chelating agents. Chelating agents, such as EDTA, can sequester metals from tissues. Chelating agents typically are ligands with plural hapticity. Once a metal is bound by a chelating agent, the chelating agent may be excreted from the body, such as through urine. The chelating agent is not particularly limited either, and includes any known chelating agent which is safe for humans, such as EDTA, EGTA, Fura-2, BAPTA, NTA, IDS, EDDS, polyaspartic acid, MGDA, L-glutamic acid, N,N-diacetic acid, GLDA, citric acid, or salts thereof.

In some embodiments, the therapeutic agent may be carried by a carrier. In general, any bulk biocompatible synthetic or natural material capable of being formed to a useful size and shape can be utilized in forming the carrier. In one embodiment, a polymeric particle can be utilized. For instance, particles formed from natural or synthetic polymers including, without limitation, polystyrene, poly(lactic acid), polyketal, butadiene styrene, styrene-acrylic-vinyl terpolymer, poly(methyl methacrylate), poly(ethyl methacrylate), poly(alkyl cyanoacrylate), styrene-maleic anhydride copolymer, poly(vinyl acetate), poly(vinyl pyridine), poly(divinylbenzene), poly(butylene terephthalate), acrylonitrile, vinyl chloride-acrylates, poly(ethylene glycol), and the like, or an aldehyde, carboxyl, amino, hydroxyl, or hydrazide derivative thereof can be utilized. Particles formed of biological polymers such as proteins can be used. For instance, particles formed of albumin (e.g., bovine serum albumin), dextran, gelatin, chitosan, dendrimers, liposomes, etc. can be utilized. Such particles can be preferred in certain embodiments as they can be formed without the use of organic solvents according to known methods. Other biocompatible materials as may be utilized in forming carrier particles can include, without limitation, oxides such as silica, titania, zirconia, and the like, and noble metals such as gold, silver, platinum, palladium, and the like. In general, the materials will be biocompatible and non-immunogenic. Suitable biodegradable materials can include, without limitation, polysaccharide and/or poly(lactic acid) homopolymers and copolymers. For example, particles formed of poly(lactic-co-glycolic acid) (PLGA) copolymers, poly(ethylene glycol) (PEG)/poly(lactic acid) (PLA) block copolymers, and derivatives thereof can be utilized.

In embodiments, the carrier may comprise a liposome. A liposome is a small vesicle having at least one lipid bilayer. In some embodiments, the liposome may comprise a singular component. In embodiments, the liposome may comprise a plurality of components, such as two components, such as three components, such as four components, such as five components, such as more than five components. For example, the liposome may comprise a lipid coupled to a polymer. Furthermore, the polymer comprise a linking group, such as a maleimide, which can allow for the liposome to become conjugated to a targeting agent, among other things. The liposome may comprise lipids including, but not limited to, SPC3, Cholesterol, DSPE-PEG and DSPE-PEG Maleimide.

The liposome carrier of the present disclosure may take several forms. Liposomes may form multilamellar vesicles (MLVs), unilamellar vesicles (ULVs), small unilamellar vesicles (SUVs), large unilamellar vesicles (LUVs), multivesicular vesicles (MVVs), or mixtures thereof. MLVs are vesicles which comprise multiple lipid bilayers, thereby forming an onion-like structure. ULVs may comprise a single lipid bilayer. SUVs are a type of ULV with a smaller size (ULVs in general can have a size of from 20 nm to 1000 nm, whereas SUVs have a size of from 20 nm to 100 nm). LUVs are a type of ULV with a larger size (LUVs have a size of from 100 to 1000). MVVs contain a plurality of vesicles on the interior of a larger vesicle.

Such different forms of vesicle may allow for the agent to have a varied concentration through the thickness of the vesicle in the case of an MLV. Such a configuration allows for a varied release of the agent as different layers of the liposome degrade in-vivo. Further, altering the size of the liposome can allow for control of degradation rate, and thereby release of the agent in-vivo.

The liposome may comprise a bulk lipid. The bulk lipid may form the bulk of the lipid bilayer, and thereby serve to give the lipid bilayer its integrity, size and thickness. In some embodiments of the present disclosure, the bulk lipid may comprise a plant-derived lipid, such as a lipid derived from a seed oil. In some embodiments, the seed oil may comprise soybean oil, canola oil, cotton oil, peanut oil, corn oil, grapeseed oil, or mixtures thereof. One such seed oil may be a modified soybean oil, such as a soybean phosphatidylcholine phospholipid (SPC3).

The liposome may comprise a stability modifier. The stability modifier can modify the stability of the liposome by having a gradual decay or uptake process in-vivo. For instance, the stability modifier may comprise a sterol, such as cholesterol, phytosterols, ergosterols, or mixtures thereof. Because the stability modifier may have effect on the rate of degradation of the liposome, the wt. % of the stability modifier, or the composition of the stability modifier itself, may be used to control the rate of release of the agent in-vivo.

The liposome may further comprise a tertiary lipid that can be conjugated to a polymer component. In embodiments, tertiary lipid may comprise a phospholipid. Said phospholipid may comprise an aminophospholipid, a type of phospholipid with an amine group bonded to the head of the phospholipid. For example, the phospholipid may comprise Distearoylphosphatidylethanolamine (DSPE). In embodiments, a fraction of the tertiary lipid may be conjugated to the polymer component. For example, a liposome may comprise a portion of the tertiary lipid that is conjugated to the polymer component, and a portion of the tertiary lipid that is not conjugated to the polymer component.

Groups that can be conjugated to the tertiary lipid may comprise a polymer component. The polymer component may serve to modulate the circulation time of the liposome and reduce its immunogenicity and antigenicity. Further, the polymer component may serve to reduce aggregation of the liposomes, thereby increasing the stability of the liposomes. In embodiments of the present disclosure, the polymer component may be conjugated to the tertiary lipid. The polymer component, without wishing to be limited, may comprise polyethylene glycol (PEG), polyvinyl alcohol (PVA), polyethylene (PE), or combinations thereof.

The liposome may, as described above, further comprise a linking group. The linking group may comprise, but is not limited to, a maleimide group, a carbodiimide, an N-hydrixysccunimide (NHS) ester, homobifunctional crosslinkers such as gluteraldehydes, or combinations thereof. The linking group may be linked to any portion of the liposome, such as the bulk lipid, the stability modifier, or the tertiary lipid.

Carbodiimide linkers may serve as a linking group by activating carboxyl groups on the surface of the liposome lipids, thereby allowing said carboxyl links to form amide bonds between the carboxyl on the surface of the liposome and an amine on a target.

NHS esters can be used to modify the surface of the liposome, which can react with an amine group on a target. NHS esters may form a bond with a terminal carboxyl group on the surface a liposome, the target thereafter forming an amide bond between itself and the liposome.

Further, the linking group may comprise a non-covalent linkage. Such a linkage may comprise Protein A-mediated conjugation. In Protein A-mediated conjugation, staphylococcal protein A can bind the Fc region of antibodies. The protein may additionally be modified to bind to the surface of a liposome. Other non-covalent linkages may comprise a biotin-streptavidin system. In the biotin-streptavidin system, the liposome may be biotinylated, and thereby allowed to bind to streptavidin. Streptavidin can be conjugated to an antibody, thereby allowing an antibody to bind to the biotin on the surface of the liposome.

The bulk lipid may be present in the liposome on a weight basis of 40 to 85 wt. %, such as between 50 wt. % to 75 wt. %, such as between 55 wt. % and 70 wt. %. The stability modifier may be present in the liposome on a weight basis of 5 wt. % to 35 wt. %, such as between 10 and 25 wt. %, such as between 15 wt. % and 25 wt. %. The tertiary lipid may be present in the liposome on a weight basis of 5 wt. % to 35 wt. %, such as between 10 and 25 wt. %, such as between 15 wt. % and 25 wt. %. Further, the liposome may comprise a conjugated tertiary lipid that is linked to a linking group, which may be present in the liposome in a wt. % that is lower than that of the tertiary lipid. The conjugated tertiary lipid that is linked to a linking group may be present in the liposome on a weight basis of 0.1 wt. % to 5 wt. %, such as between 0.5 wt. % to 2 wt. %.

Selection of the carrier, such as the liposome, can be utilized to provide control of release rate of a biologically active agent from the liposome. For instance, selection of a biodegradable material, such as the stability modifier, can be utilized to control the rate of agent release and provide a release mechanism that can be controlled to a large extent by particle degradation rate and to a lesser extent by diffusion of the active agent through and out of the bulk particle. Materials can be utilized such that active agent release rate is limited by one of diffusion (e.g., a nondegradable carrier) or carrier degradation rate (e.g., essentially no diffusion of the active agent through the particle due to small matrix mesh size), or to some combination thereof that can be engineered for a desired release rate.

Particles can be microparticles or nanoparticles. As utilized herein, the term nanoparticle generally refers to a particle of which the size, i.e., the average diameter, can be about 1000 nanometers (nm) or less, generally about 500 nm or less, for instance about 200 nm or less, or about 100 nm or less. In one particular embodiment, nanoparticles can be about 50 nm or less in size, for instance about 20 nm in average diameter. In one embodiment, nanoparticles can have an average diameter of from about 50 nm to about 400 nm, or from about 100 nm to about 300 nm.

Larger particles can alternatively be utilized. For instance, in other embodiments, microparticles having an average size of up to about 50 micrometers (μm) can be utilized as a carrier.

In general, the preferred size of particles can depend upon the specific application, e.g., the specific method of delivery of the agents, such as via surface application (as in a cream or lotion), via parenteral injection using the circulatory or digestive tract, via inhalation, etc., as well as the desired release rate of an agent from the particles. For instance, particles can be of a size to prevent cellular uptake so as to remain in the extracellular matrix and available for interaction with damaged elastic fibers. Thus, the particles may be about 100 nm or larger in one embodiment, as smaller particles have been shown to exhibit higher cellular uptake. Particles can also be small enough so as to penetrate endothelium and penetrate basement membrane so as to contact the elastic fibers of the connective tissue. For instance, particles can be about 400 nm or less in average diameter in one embodiment so as to penetrate endothelium and basement membrane. When intended for use in an intravenously administered formulation, large particles (e.g., greater than about 1 μm) are typically disfavored because they can become lodged in the microvasculature. In addition, larger particles can accumulate or aggregate in vivo. As such, for intravenous administration, particles under 1 μm are typically used.

As will be appreciated by those skilled in the art, the composition, shape, size, and/or density of the particles may vary widely. In embodiments wherein the carrier comprises a liposome, the liposome may be generally spherical.

When utilizing a single-step formation process, an agent for delivery (e.g., a therapeutic) can also be included in either a first solution or a second solution. Upon formation of the particles, the agent can be incorporated in the particles with the bulk material.

Initial concentration of an agent within or on a particle will vary depending upon the nature of the agent, delivery rate, etc. For example, in one embodiment, loading concentration of a biologically active agent in/on a particle can vary from about 4 wt. % to greater than about 60 wt. % by weight of the particle, with higher and lower concentrations possible depending upon specific agent, particle bulk material, and the like. For instance, in an embodiment in which an agent for delivery exhibits high solubility in the bulk particle material, a very high loading level can be attained, particularly when both materials are highly hydrophobic.

Formation processes can include two-step processes in which particles are first formed followed by a second loading step in which one or more active agents are loaded into the formed particles or onto the surface of the formed particles. Formation processes may vary depending on the desired carrier for the final product. For instance, should the carrier comprise a liposome, a formation process may comprise mixing a first fluid stream containing liposome precursors with a second fluid stream carrying the agent. Said mixing may be accomplished in a variety of ways, as will be described below, but include, among other methods, mixing the fluid streams in a microfluidic device, such as a microfluidic “Y” junction, a microfluidic “T” junction or a coaxial flow microfluidic device. The mixing of the liposome precursors with the second fluid stream may cause the liposome precursor to self-assemble into micelles. The interior of the micelles, or liposomes, may comprise a fluid as stated above. The fluid may comprise an active agent.

After formation of the liposomes, the combined fluid streams form a liposome solution may undergo continuous stirring. During said stirring step, targeting peptides or proteins may be added to the liposome solution. The targeting peptides or proteins may be functionalized with a moiety which can conjugate with a component on the surface of the liposome. For instance, the targeting peptides or proteins may be sulfonated, which can then conjugate with the linking group present on the surface of the liposome. After the formation of the liposomes, or after their functionalization with a targeting peptide or protein, the liposomes may be filtered or washed in order to remove any free agents or targeting proteins or peptides. For instance, the targeting-labeled liposomes may be filtered, such as by using tangential flow, to separate any unconjugated targeting peptides or proteins or agent.

The first fluid stream may comprise liposome components in a solvent including, but not limited to, alcohols such as ethanol, propanol, isopropyl alcohol, ethers such as ether, diethyl ether, methyl ethyl ether, sulfones such as DMSO, ketones such as acetone or methyl ethyl ketone, or mixtures thereof. Generally, the first fluid stream may comprise an organic solvent.

Included in the first fluid stream can be the liposome components or liposome precursors as described above. Without wishing to be bound to any particular theory, the use of an organic solvent as the fluid in which liposomes are formed can aid in their stabilization during formation.

The liposomes or liposome precursors may be present in the first fluid stream at a concentration of 0.01 to 100 millimolar, such as between 0.1 millimolar and 20 millimolar, such as between 1 millimolar and 10 millimolar.

The flow rate of the first fluid stream may be between 5 and 150 milliliters per minute, such as between 10 and 100 milliliters per minute, such as between 20 and 90 milliliters per minute, such as between 35 and 85 milliliters per minute.

The second fluid stream may comprise an aqueous phase comprising water. The agent may be dissolved in or dispersed in the second fluid stream. Further, the second fluid stream may comprise a buffer.

The agent may have a concentration in the second fluid stream of between 10 millimolar and 1000 millimolar, such as between 20 millimolar and 600 millimolar, such as between 80 and 400 millimolar.

The buffer, when present, may comprise, but is not limited to, a PB buffer, a PBS buffer, a HEPES buffer, a Tris (hydroxymethyl) aminomethane buffer, a carbonate buffer or mixtures thereof. The buffer can have a concentration in the second fluid stream of between 1 and 40 millimolar, such as between 3 and 20 millimolar, such as between 5 and 15 millimolar.

The flow rate of the second fluid stream may be between 10 milliliters per minute and 200 milliliters per minute, such as between 20 milliliters per minute and 150 milliliters per minute, such as between 50 milliliters per minute and 120 milliliters per minute.

While the above description lists various flow rates for both of the first fluid stream and the second fluid stream, the present inventors have found that the ratio of the two flow rates may impact liposome formation and loading. Thus, the present method contemplates a ratio of flow rates of the first fluid stream to the second fluid stream of between 0.5 to 5 and 5 to 6, such as between 0.75 to 4 and 4 to 5, such as between 1 to 4 and 2 to 3, such as 1.5.

Furthermore, in embodiments of the present disclosure, the first fluid stream may have the flow rates as described above through a tube with an inner diameter of between 1/64^{th} of an inch and ¼^{th} of an inch, such as between 1/48^{th} of an inch and 1/10^{th} of an inch, such as between 1/32^{nd} of an inch and 1/16^{th} of an inch. Similarly, the second fluid stream may have the flow rates as described above through a tube with an inner diameter of between 1/64^{th} of an inch and ¼^{th} of an inch, such as between 1/48^{th} of an inch and 1/10^{th} of an inch, such as between 1/32^{nd} of an inch and 1/16^{th} of an inch. In some embodiments, second fluid stream may the flow rates as described above through a tube with an inner diameter of between 1/10^{th} and 1/32^{nd} of an inch.

The device in which mixing occurs, such as the T mixer, Y mixer or coaxial flow microfluidic device described above, may have a mix tube diameter of between ¼^{th} of an inch and 1/64^{th} of an inch, such as between ⅙^{th} of an inch and 1/48^{th} of an inch, such as between ⅛^{th} of an inch and 1/32^{nd} of an inch, such as ⅙^{th} of an inch.

Further, the intensity of the stirring in the targeting protein or peptide conjugation step may be adjusted to form liposomes of alternate sizes. For example, when a stirring rate of 300 rpm is used, liposomes with sizes of 80 to 215 nanometers may be formed. The polydispersity index, which is a measure of the breadth of distribution of sizes around a mean, may be between 0.15 and 0.5, such as between 0.2 and 0.35.

Loaded carriers can be formed so as to control the rate of release of active compound from a particle. Suitable control mechanisms are known to those of skill in the art. For instance, release rates can depend upon the relative concentration of an agent for delivery to bulk carrier material, upon the molecular weight and degradation characteristics of the bulk carrier material. In any of these cases, one of ordinary skill in the art is capable of engineering a system so as to achieve desirable release rate. For instance, in the case of purely diffusion-limited release, such control can be achieved by variation of agent concentration within particles and/or particle size. Agent concentration within particles, particularly within liposomes as described above, can be controlled by variation of the concentration of the agent in the second fluid stream. Release rate of an agent from particles can be adjusted utilizing the above parameters so as to produce carriers capable of sustained release for periods varying from a few days to a few months, with the maximum release rates generally varying from a few hours to a few weeks.

In embodiments of the present disclosure, the liposomes may have an agent loading of greater than 20%, such as greater than 40%, such as greater than 60%. Furthermore, the liposomes may, when conjugated to a targeting peptide or protein, have percent of antibody conjugation greater than 80%, such as greater than 85%, such as greater than 90%. Furthermore, liposomes may undergo filtration and/or separation to increase one or both of the loading % or conjugation %.

Loading of liposomes may refer to the content of the agent within the liposome, such as on a weight basis. Loading content may be measured by drying liposomes and measuring the total weight of the dried liposomes. The liposomes may then be re-hydrated and heating, so as to disrupt the lipid bilayers of the liposomes. The amount of agent in the sample may then be measured, and compared against the total weight of the liposomes and agent. Further, while liposomes can be formed and loaded simultaneously as in above, other methods exist for loading liposomes with an agent. Loading of the agent into liposomes can be achieved through various methods, broadly categorized as passive or active, including techniques like thin-film hydration, detergent depletion, and emulsion methods, each with advantages for specific drug types and loading efficiencies. In the thin-film hydration method, a thin lipid film is created by evaporating a lipid-solvent solution, followed by hydration of the lipid film with an aqueous solution containing the agent. In the detergent depletion method, lipids are solubilized with a solution comprising a detergent and the agent to form lipid-detergent micelles, followed by detergent removal, leading to the formation of homogeneous liposomes. In the emulsion method, a water in oil emulsion is transferred to a large aqueous solution and agitated to form a double emulsion (water in oil in water), wherein the agent is dissolved in either of the first or second sets of water, or the oil. In the mechanical dispersion method, sonication or extrusion is used to create small-sized liposomes, suitable for both hydrophilic and hydrophobic drugs. The supercritical anti-solvent (SAS) method can be used for for the preparation of proliposomes, offering a simple approach with low solvent residue and is suitable for drugs with low solubility in the SCFs. Similarly, the polyol dilution method can be used for the mass production of liposomes. The active drug-loading approach involves creating a concentration gradient (e.g., pH or ion gradient) to force drug molecules into the liposome. The injection method involves injecting the liposomes with the drug into the liposome after they are formed. The dilution method involves diluting concentrated dispersions of liposomes with different concentrations of drug solutions. Agents can also be conjugated to the surface of a liposome, similar to how targeting proteins or peptides may be. Agents for delivery need not necessarily be incorporated within the liposome. For example, in one embodiment, an agent can be bonded to the surface of a particle. For example, an agent can be bonded to the surface of a particle utilizing chemistry similar to that as is described in more detail below with regard to the binding of the epitope binding antibodies or fragments to the particles.

In embodiments of the present disclosure, the liposome may have a negative surface charge. Such a negative surface charge may be determined by zeta potential analysis.

Selection of bulk carrier material can be utilized to provide control of release rate of a biologically active agent from the loaded particle. For instance, selection of a biodegradable material can be utilized to control the rate of agent release and provide a release mechanism that can be controlled to a large extent by particle degradation rate and to a lesser extent by diffusion of the active agent through and out of the bulk particle. Materials can be utilized such that active agent release rate is limited by one of diffusion (e.g., a nondegradable particle) or nanoparticle degradation rate (e.g., essentially no diffusion of the active agent through the particle due to small matrix mesh size), or to some combination thereof that can be engineered for a desired release rate.

Particles can be microparticles or nanoparticles. As utilized herein, the term nanoparticle generally refers to a particle of which the size, i.e., the average diameter, can be about 1000 nanometers (nm) or less, generally about 500 nm or less, for instance about 200 nm or less, or about 100 nm or less. In one particular embodiment, nanoparticles can be about 50 nm or less in size, for instance about 20 nm in average diameter. In one embodiment, nanoparticles can have an average diameter of from about 50 nm to about 400 nm, or from about 100 nm to about 300 nm.

Larger particles can alternatively be utilized. For instance, in other embodiments, microparticles having an average size of up to about 50 micrometers (μm) can be utilized as a carrier.

In general, the preferred size of particles can depend upon the specific application, e.g., the specific method of delivery of the agents, such as via surface application (as in a cream or lotion), via parenteral injection using the circulatory or digestive tract, via inhalation, etc., as well as the desired release rate of an agent from the particles. For instance, particles can be of a size to prevent cellular uptake so as to remain in the extracellular matrix and available for interaction with damaged elastic fibers. Thus, the particles may be about 100 nm or larger in one embodiment, as smaller particles have been shown to exhibit higher cellular uptake. Particles can also be small enough so as to penetrate endothelium and penetrate basement membrane so as to contact the elastic fibers of the connective tissue. For instance, particles can be about 400 nm or less in average diameter in one embodiment so as to penetrate endothelium and basement membrane. When intended for use in an intravenously administered formulation, large particles (e.g., greater than about 1 μm) are typically disfavored because they can become lodged in the microvasculature. In addition, larger particles can accumulate or aggregate in vivo. As such, for intravenous administration, particles under 1 μm are typically used.

Generally, particulate carriers can be substantially spherical in shape, although other shapes including, but not limited to, plates, rods, bars, irregular shapes, etc., are suitable for use. As will be appreciated by those skilled in the art, the composition, shape, size, and/or density of the particles may vary widely.

When utilizing a single-step formation process, an agent for delivery (e.g., a therapeutic) can also be included in either the first solution or the second solution. Upon formation of the particles, the agent can be incorporated in the particles with the bulk material.

Initial concentration of an agent within or on a particle will vary depending upon the nature of the agent, delivery rate, etc. For example, in one embodiment, loading concentration of a biologically active agent in/on a particle can vary from about 4 wt. % to about 40 wt. % by weight of the particle, with higher and lower concentrations possible depending upon specific agent, particle bulk material, and the like. For instance, in an embodiment in which an agent for delivery exhibits high solubility in the bulk particle material, a very high loading level can be attained, particularly when both materials are highly hydrophobic. In embodiments of the present disclosure, the concentration of the agent may be from 8 wt. % to 32 wt. % by weight of the particle, such as from 15 wt. % to 28 wt. % by weight of the particle.

Formation processes can include two-step processes in which particles are first formed followed by a second loading step in which one or more active agents are loaded into the formed particles or onto the surface of the formed particles. For instance, a method can include swelling a pre-formed, optionally crosslinked, polymeric particle in a solution that includes the agent for delivery so as to load the particle via a diffusion process. In another embodiment, loading method can include double emulsion polymerization, which enables loading of hydrophilic compounds into hydrophobic particles. The formation method for nanoparticles is not particularly limited and other formation methods as are known in the art, e.g., sonication methods, solvent precipitation methods, etc., may be utilized.

Loaded particles can be formed so as to control the rate of release of active compound from a particle. Suitable control mechanisms are known to those of skill in the art. For instance, release rates can depend upon the relative concentration of an agent for delivery to bulk particle material, upon the molecular weight and degradation characteristics of the bulk nanoparticle material, upon the mesh size of a polymer particle matrix, upon the binding mechanism between the surface of a particle and an agent, and so forth, as is known. In any of these cases, one of ordinary skill in the art is capable of engineering a system so as to achieve desirable release rate. For instance, in the case of purely diffusion-limited release, such control can be achieved by variation of agent concentration within particles and/or particle size, particle polymer mesh size, and so forth. In the case of purely degradation-limited release, polymer monomer units, for instance glycolic acid content of a PLGA polymer, and/or molecular weight of particle bulk material, as well as particle size, can be adjusted to “fine tune” active compound release rate. For example, use of PLGA polymers with higher glycolic acid content and lower molecular weight can lead to an increased degradation rate of a particle formed with the polymer. Release rate of an agent from particles can be adjusted utilizing the above parameters so as to produce carriers capable of sustained release for periods varying from a few days to a few months, with the maximum release rates generally varying from a few hours to a few weeks.

Agents for delivery need not necessarily be incorporated within the bulk material. For example, in one embodiment, an agent can be bonded to the surface of a particle. For example, an agent can be bonded to the surface of a particle utilizing chemistry similar to that as is described in more detail below with regard to the binding of the epitope binding antibodies or fragments to the particles.

Further, the surface of the particle may comprise targeting peptides or proteins, such as antibodies. In one embodiment described below, a particular therapeutic target may be damaged elastin, such as is found in calcified blood vessels. However, in general, the present disclosure is broadly applicable to a variety of therapeutic targets which can be targeted in some fashion, particularly as through a peptide or protein. For instance, other targets may include neural tissues, connective tissues, or fatty tissues.

For example, in one embodiment, the antibody may comprise an anti-elastin antibody. In some embodiments, the disclosed antibodies and antigen binding fragments specifically recognize and bind an epitope sequence of one or more of GALGPGGKPPKPGAGLL (SEQ ID NO: 1), LGYPIKAPKLPGGYGLPYTTGKLPYGYPGGVAGAAGKAGYPTTGTGV (SEQ ID NO: 2), or PGGYGLPYTTGKLPYGYP (SEQ ID NO: 3). Also disclosed are delivery agents that can incorporate the anti-elastin antibodies and antigen binding fragments thereof as targeting agents for delivery of biologically active agents to an area that includes elastin.

The epitope sequences exemplified by SEQ ID NOs: 1-3 are polypeptide components of the amorphous, crosslinked elastin component of an elastic fiber that can become exposed and accessible upon degradation of the elastic fiber, and in particular, upon degradation of the microfibril scaffolding structures of elastic fibers. As such, in one embodiment, the disclosed targeting agents can be utilized to bind to damaged elastic fibers and can exhibit little or no binding to healthy elastic fibers or soluble elastin precursors or break-down components as may circulate in the blood. For instance, a targeting agent that includes an antibody or antigen binding fragment(s) thereof that specifically recognizes and binds one or more of SEQ ID NOs: 1-3 can exhibit little or no binding to alpha-elastin degradation products. In one embodiment, targeting agents can bind immature elastin that is no longer soluble but that is not fully crosslinked and formed as elastic fibers, e.g., immature elastin in atherosclerotic fibrous caps.

The disclosed antibodies/fragments encompass immunoglobulin molecules and immunologically active portions of immunoglobulin molecules (i.e., molecules that contain an antigen binding site that immuno-specifically bind one or more of the polypeptides described herein). A complete antibody can generally be comprised of two immunoglobulin heavy chains and two immunoglobulin light chains. In one particular embodiment, an antibody as disclosed herein can include as heavy chain SEQ ID NO: 5 and as light chain SEQ ID NO: 23. However, it should be understood that the invention encompasses complete antibodies that include the variable portions of the disclosed antibodies (SEQ ID NO: 7 (V_{H}) and SEQ ID NO: 25 (V_{L})) in conjunction with alternative constant regions, as well as isolated antigen binding portions thereof (e.g., one or more CDR regions SEQ ID NOs: 9, 11, 13, 27, 29, and 31, optionally in conjunction with their respective FR regions SEQ ID NOs: 15, 17, 19, 21, 33, 35, 37, 39). Targeting agents disclosed herein based upon the disclosed antibodies can include, without limitation, an immunoglobulin molecule, a monoclonal antibody, a polyclonal antibody, a chimeric antibody, a CDR-grafted antibody, a non-human antibody (e.g., from mouse, rat, goat or any other animal), a fully-human antibody, a humanized antibody, a Fab, a Fab′, a F(ab′)2, a Fv, a disulfide-linked Fv, a scFv, a single-domain antibody based on either a heavy chain variable domain or a light chain variable domain (a nanobody), a diabody, a multispecific antibody, a dual-specific antibody, an anti-idiotypic antibody, a bispecific antibody, a functionally active epitope-binding fragment thereof, bifunctional hybrid antibodies, a single chain of an antibody, etc. An antibody may be of any type (e.g., IgG, IgA, IgM, IgE, or IgD). In general, the antibody is an IgG, e.g., an IgG1, IgG2, or an IgG3 isotype. In one particular embodiment, an antibody can be an IgG1 isotype. In addition, an antibody can generally include kappa light chains.

Antigen binding compounds as disclosed herein are not limited to complete antibodies. In one embodiment, disclosed compounds and methods can utilize one or more antigen binding fragments of a complete antibody. For instance, methods and materials can incorporate one or more CDR regions of a full antibody that can target and bind an epitope of elastin. By way of example, a targeting agent can include one or more of SEQ ID NO: 9, SEQ ID NO: 11 or SEQ ID NO: 13, which describe CDR fragments of a variable region of a heavy chain (SEQ ID NO: 7) as described herein, optionally in conjunction with one or more of SEQ ID NO: 27, SEQ ID NO: 29, or SEQ ID NO: 31, which describe CDR fragments of a variable region of a light chain (SEQ ID NO: 25) as described herein. A CDR fragment can be provided in one embodiment bounded by one or both FR fragments as found in a complete variable region, or alternatively, can be utilized in an isolated format, independent of the natural FR fragments. By way of example, in one embodiment, a targeting agent as described herein can incorporate a peptide sequence including SEQ ID NOs: 15, 9, and 17, in sequential order, which includes a CDR fragment (SEQ ID NO: 9) of a monoclonal antibody described herein in conjunction with the FR fragments naturally found on either end of the CDR fragment (SEQ ID NO: 15 and SEQ ID NO: 17). FR fragments that can be utilized in conjunction with CDR fragments can include one or more of SEQ ID NOs: 15, 17, 19, 21, 33, 35, 37, and 39 in formation of a targeting agent that selectively recognizes an epitope of degraded elastin.

As utilized herein, the terms “selectively recognizes” and “selectively binds” mean that binding of the molecule to an epitope is 2-fold greater or more, for instance from about 2 fold to about 5 fold greater, than the binding of the molecule to an unrelated epitope or than the binding of an unrelated molecule to the epitope, as determined by techniques known in the art, such as, for example, ELISA, immunoprecipitation, two-hybrid assays, cold displacement assay, etc. Typically, specific binding can be distinguished from non-specific binding when the dissociation constant (K_{D}) is about 1×10⁻⁵ M or less, or about 1×10⁻⁶ M or less, for instance about 1×10⁻⁷ M in some embodiments.

In some embodiments, functional antigen binding fragments of the disclosed antibodies can include Fab, a scFv-Fc bivalent molecule, F(ab′)2, and Fv that are capable of specifically recognizing and binding with one or more of SEQ ID NOs: 1-3, e.g., one or more of SEQ ID NOs: 7, 9, 11, 13, 25, 27, 29, or 31.

Antigen binding peptides as described herein can incorporate modifications as would be understood by one of skill in the art. For instance, there are many natural amino acids, which occur as L-isomers in most living organisms; however, embodiments of the disclosure are not limited to only L-amino acids and can include modifications that substitute D-amino acids or other non-proteinogenic amino acids that are not naturally encoded by humans or any other organism. Herein, unless specifically referenced as a D-amino acid (i.e., the amino acid identifier followed by (d)), reference to a generic amino acid indicates the L-amino acid.

In embodiments of the disclosure, a targeting agent can include an ornithine substitution to disclosed peptides, e.g., to disclosed CDR fragments as may be utilized in a targeting agent. In some embodiments, a targeting agent can include one or more amino acid substitutions of a human proteinogenic amino acids selected from the following group: alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, and valine.

In one embodiment, a targeting agent can include structurally and/or functionally similar peptides to those disclosed herein. Structurally similar peptides can encompass variations such as the substitution of one amino acid having a first amino acid side chain with a second amino acid having a second amino acid side chain. Both the first amino acid side chain and the second amino acid side chain provide a similar characteristic to maintain functional similarity of the targeting agent, i.e., elastin epitope binding. A similar characteristic can include a side chain that has a similar polarity, charge, or size as the first amino acid side chain. As an example, leucine includes a hydrophobic side chain, and in some embodiments, a targeting agent can include substitution of a leucine of a disclosed sequence (e.g., a CDR sequence) with an isoleucine, valine, or alanine, as each of these amino acids includes a similar hydrophobic side chain. As another example, histidine includes an aromatic side chain that can also carry a positive charge, and in some embodiments, one or more histidines of an elastin binding antibody or fragment thereof can be substituted with an amino acid that includes an aromatic side chain or with an amino acid that can carry a positive charge, such as phenylalanine, tyrosine, tryptophan, arginine, or lysine. These are provided as examples of possible substitutions and are not meant to limit the scope of variations contemplated by substituting amino acids that have similar side chain properties.

In some embodiments, the antigen binding fragments comprise a Fab, in which the fragment contains a monovalent antigen binding fragment of the antibody molecule, and which can be produced by digestion of whole antibody with the enzyme papain to yield an intact light chain (e.g., SEQ ID NO: 23) or the variable region thereof (e.g., SEQ ID NO: 25) and a portion of one heavy chain (e.g., one or more of SEQ ID NO: 9, 11, 13, optionally in conjunction with one or more of SEQ ID NOs: 15, 17, 19, 21).

In one embodiment, the antigen binding fragment can comprise a Fab′, which is the fragment of the antibody molecule that can be obtained by treating whole antibody with pepsin, followed by reduction, to yield an intact light chain (e.g., SEQ ID NO: 23) or the variable region thereof (e.g., SEQ ID NO: 25) and a portion of the heavy chain (e.g., one or more of SEQ ID NO: 9, 11, 13, optionally in conjunction with one or more of SEQ ID NOs: 15, 17, 19, 21); two Fab′ fragments can be obtained per antibody molecule. A (Fab′)2 fragment of the antibody is encompassed, which can be obtained by treating a whole antibody with the enzyme pepsin without subsequent reduction. A F(ab′)2 fragment is a dimer of two Fab′ fragments held together by two disulfide bonds. Also encompassed is a Fv, which is a genetically engineered fragment containing the variable region of the light chain and the variable region of the heavy chain expressed as two chains. In one embodiment, the antibody can encompass a single chain antibody (“SCA”), which is a genetically engineered molecule containing the variable region of the light chain and the variable region of the heavy chain, linked by a suitable polypeptide linker as a genetically fused single chain molecule. An antibody fragment can be an scFv-Fc, which is produced in one embodiment by fusing single-chain Fv (scFv) with a hinge region from an immunoglobulin (Ig), such as an IgG, and Fc regions.

An antibody or antigen binding fragment thereof can include a modification as is known in the art that does not interfere with the specific recognition and binding with the targeted epitope. For instance, a modification can minimize conformational changes during the shift from displayed to secreted forms of the antibody or fragment. As is understood by a skilled artisan, the modification can be a modification known in the art to impart a functional property that would not otherwise be present if it were not for the presence of the modification. The invention encompasses materials that are differentially modified during or after translation, e.g., by glycosylation, acetylation, phosphorylation, amidation, derivatization by known protecting/blocking groups, proteolytic cleavage, linkage to a particle, another molecule or other cellular ligand, etc. Any of numerous chemical modifications may be carried out by known techniques, including, but not limited to, specific chemical cleavage by cyanogen bromide, trypsin, chymotrypsin, papain, V8 protease, NaBH₄, acetylation, formylation, oxidation, reduction, metabolic synthesis in the presence of tunicamycin, etc.

A modification can include a N-terminus modification and/or a C-terminal modification. For example, the modification can include a N-terminus biotinylation and/or a C-terminus biotinylation. In one embodiment, the secretable form of the antibody or antigen binding fragment comprises a N-terminal modification that allows binding to an Immunoglobulin (Ig) hinge region. In another embodiment, the Ig hinge region is from, but is not limited to, an IgA hinge region. In another embodiment, the secretable form of the antibody or antigen binding fragment comprises a N-terminal modification and/or a C-terminal modification that allows binding to an enzymatically biotinylatable site. In another embodiment, biotinylation of said site can functionalize the site to bind to any surface coated with streptavidin, avidin, avidin-derived moieties, or a secondary reagent.

A modification can include, for example, addition of N-linked or O-linked carbohydrate chains, attachment of chemical moieties to the amino acid backbone, chemical modifications of N-linked or O-linked carbohydrate chains, and addition or deletion of a N-terminal methionine residue.

The antibodies or antigen binding fragments can be produced by any synthetic or recombinant process such as is well known in the art. The antibodies or antigen binding fragments can further be modified to alter biophysical or biological properties by means of techniques known in the art. For example, an antibody can be modified to increase its stability against proteases, or to modify its lipophilicity, solubility, or binding affinity to one or more of SEQ ID NOs: 1-3.

By way of example, the antibodies can be produced by the immunization of various animals, including mice, rats, rabbits, goats, primates, chickens and humans with a target antigen such as an entire peptide sequence as described or a peptide fragment of elastin containing one or more of the sequences as described that include at least one anti-elastin epitope. In one embodiment, the antigen or peptide fragment containing the antigen can be purified prior to immunization of the animal. The antibody or antigen binding fragment obtained following the immunization can be purified by methods known in the art, for example, gel filtration, ion exchange, affinity chromatography, etc. Affinity chromatography or any of a number of other techniques known in the art can be used to isolate polyclonal or monoclonal antibodies from serum, ascites fluid, or hybridoma supernatants.

“Purified” means that the antibody is separated from at least some of the proteins normally associated with the antibody and preferably separated from all cellular materials other than proteins.

The antibodies or antigen binding fragments thereof may be produced by using gene recombination techniques. For example, in formation of a chimeric antibody, a humanized antibody, a functional fragment of antibody or the like, such as a Fv, a SCA, a scFv-Fc or the like, genetic recombination techniques.

In one embodiment, a method for producing a targeting agent that incorporates all or a portion of a variable region of a heavy chain (SEQ ID NO: 7) and a variable region of a light chain (SEQ ID NO: 25), e.g., including one or more CDR regions (SEQ ID NOs: 9, 11, 13, 27, 29, 31), for instance in formation of a chimeric antibody, can be carried out through utilization of genetic recombination techniques.

By way of example, DNA encoding an amino acid sequence (V_{H} region) represented by SEQ ID NO: 7 is prepared. Likewise, DNA encoding an amino acid sequence (V_{L}) represented by SEQ ID NO: 25 is prepared. Examples of such DNA include those represented by SEQ ID NO: 6 and SEQ ID NO: 24; however, those having other nucleotide sequences may be used.

Portions or mutants of disclosed sequences, which still retain desired activity, are also considered within the scope of this disclosure. For example, mutants can include alterations to SEQ ID NO: 6 or SEQ ID NO: 24 that encode one or more amino acid substitutions (e.g., mutating a codon for valine to a codon for alanine). Additionally, or alternatively, mutants of a DNA sequence can include one or more point mutations to the native cDNA sequence to substitute a degenerate codon for the native codon.

For embodiments of the disclosure that include a mutant of a nucleic acid sequence as disclosed (e.g., SEQ ID NO: 6 or SEQ ID NO: 24 or portions thereof encoding a CDR region of an antibody), the mutant can include one or more codon mutations that modify the expressed protein to substitute one hydrophobic amino acid (e.g., valine) for another hydrophobic amino acid (e.g., alanine, leucine, isoleucine, proline, phenylalanine, methionine, or tryptophan) to produce an antibody variant.

Due to codon redundancy, there are many theoretically possible cDNA sequence variants that could encode an antibody or antigen binding fragment as described herein. Additionally, variants that modify the native protein sequence, while retaining binding activity, further increase this number. For these embodiments, a genetic modification can result in the expression of a peptide (e.g., SEQ ID NO: 7) or a peptide variant that retains the binding function of the native peptide.

A DNA encoding V_{H} (e.g., SEQ ID NO: 7) or V_{L} (e.g., SEQ ID NO: 25) can be inserted into a vector having a sequence encoding the respective constant regions (C_{H} or C_{L}) of human antibody in one embodiment to construct a chimeric antibody expression vector. Vectors having a sequence encoding C_{H} or C_{L} of a human antibody as may be utilized are commercially available. By introducing the constructed expression vector into a host cell, a recombinant cell that expresses a chimeric antibody can be obtained. Following, the recombinant cell can be cultured, and a desired chimeric antibody can be acquired from the culture.

A host cell is not particularly limited as long as the expression vector is able to function therein. By way of example, animal cells (e.g., COS cells, CHO cells, HEK cells, and the like), yeast, bacteria (Escherichia coli and the like), plant cells, insect cells and the like may be appropriately employed.

In one embodiment, a recombination technique can be utilized to produce an antibody including specific CDR including one or more of SEQ ID NOs: 9, 11, 13, 27, 29, or 31. For instance, a method can be utilized in forming a humanized antibody, which, as utilized herein, refers to an antibody having a CDR derived from an animal other than human, and other regions (framework region, constant region and the like) derived from human.

For example, nucleotide sequences encoding heavy chain CDRs (SEQ ID NOs: 9, 11, 13) and light chain CDRs (SEQ ID NOs: 27, 29, 31) of an antibody can be prepared. As the DNA, a sequence corresponding to each CDR nucleotide sequence represented by SEQ ID NOs: 8, 10, 12, 26, 28, 30 is exemplified; however, as discussed above, those having other nucleotide sequences may be used. DNA may be prepared by known methods such as PCR. The DNA may be prepared by chemical synthesis. SEQ ID NO: 12 may have a nucleotide sequence of gaagactac. SEQ ID NO: 13 may have an amino acid sequence of Glu Asp Tyr.

Using these sequences, a sequence encoding a variable region in which heavy chain CDR encoding regions (e.g., SEQ ID NOs: 8, 10, 12) are grafted to the respective regions encoding framework regions (FR) of V_{H} in a human antibody can be prepared. Likewise, sequences encoding a variable region in which light chain CDR encoding regions (e.g., SEQ ID NOs: 26, 28, 30) are grafted to the respective regions encoding FR of V_{L} in a human antibody can be prepared. The prepared nucleic acid sequence can then be inserted into a vector having a sequence encoding the desired constant region (C_{H} or C_{L}) of a human antibody, so as to construct a humanized antibody expression vector. By introducing the constructed expression vector into a host cell, a recombinant cell that expresses a humanized antibody can obtained. The recombinant cell can then be cultured, and a desired humanized antibody can be acquired from the culture.

A targeting agent including fewer than all of the CDRs of a full antibody can be produced in a similar procedure. For instance, a targeting agent that includes only the V_{H} or only the V_{L} region of an antibody, absent the constant region, can be produced in a similar fashion.

Methods for purifying a targeting agent formed according methods as described herein are not particularly limited and known techniques may be employed. For example, a culture supernatant of a hybridoma or a recombinant cell may be collected, and the antibody or antigen binding fragment may be purified by a combination of known techniques such as various kinds of chromatography, salt precipitation, dialysis, membrane separation and the like. When the isotype of the antibody is IgG, the antibody may be conveniently purified by affinity chromatography using protein A.

In utilization of disclosed materials, an antibody or antigen binding fragment can be operably linked to a secondary material for targeting and delivery of an agent to a degraded elastic fiber or to an area near a degraded elastic fiber. As utilized herein, the term “operably linked” refers to a direct or indirect linkage that can be either a permanent or temporary (e.g., degradable) linkage in which two or more molecules, sequences, particles or combination thereof are attached in such a manner as to ensure the proper function of the components, and in particular, in such a manner that the antibody or antigen binding fragment thereof can bind its epitope. As such, the antibodies or antigen binding fragment thereof can deliver any kind of useful agent to areas in or near connective tissues such as arteries, lungs, skin, etc. Moreover, in some embodiments, an antibody or antigen binding fragment can be directly linked to a carrier (e.g., a particle as described further herein) that can carry and deliver one or more active agents. As such, a composition can be utilized to deliver an active agent over an extended time period via controlled release of the agent from the carrier.

The antibodies or antigen binding fragments thereof can be utilized for delivery of biologically active agents in treatment or diagnosis of diseases for which elastin protein degradation is a hallmark including cardiovascular diseases, such as atherosclerosis and arteriosclerosis, and lung diseases, such as chronic bronchitis, COPD, and emphysema. Other conditions that can include elastic fiber degradation and for which the antibodies or antigen binding fragments thereof can be utilized in agent delivery can include those associated with aneurysm, arteriosclerosis, atherosclerosis, genetic disorders, blunt force injury, Marfan's syndrome, pseudoxanthoma elasticum, skin aging, and so forth. In one embodiment, the materials can be utilized for treatment of vascular calcification which is common in aging, as well as in a number of genetic and metabolic disorders. Vascular calcification is now recognized as a strong predictor of cardiovascular events in those suffering from other disorders such as in diabetes and chronic kidney disease (CKD), as well as in the general population. The materials can be utilized in treatment of medial arterial calcification (MAC), which can exist independently of atherosclerosis and is typically associated with elastic fiber degradation. Elastin-specific medial calcification leads to an elevation of systolic blood pressure (SBP) and pulse pressure (PP) and contributes to isolated systolic hypertension (ISH). In one embodiment, disclosed materials can be utilized in targeting immature and/or damaged elastin fiber simultaneously in intimal and medial calcification. For instance, when both atherosclerotic and medial calcification are present in a subject, disclosed materials can target by calcifications simultaneously.

In one embodiment, disclosed materials and methods can show benefit in stabilizing vulnerable atherosclerotic plaque. Atherosclerotic plaques have been found to include a fibrous cap that is produced over the plaque. It has recently been discovered that these fibrous caps can include immature (i.e., not fully crosslinked and formed). Currently research shows that some patients have stable plaques with thick fibrous cap, and some have a vulnerable thin cap. Rupture of plaque due to the presence of a relatively thin cap can lead to death. Disclosed antibodies can bind the immature elastin in these atherosclerotic fibrous caps and thereby assist in delivering bioactive agents to the local area, e.g., in conjunction with carrier nanoparticles. For example, agents that can stabilize collagen/elastin of the fibrous cap or that can otherwise increase the strength of the cap and prevent rupture can be delivered by use of the targeting antibodies.

The materials may have application in skin care, such as for conditions including scarring, skin sagging and wrinkles, which often occur with age due to loss/degradation of elastic fiber including that due to sun exposure or other disease states. Patients as may benefit from utilization of the delivery agents can also include those suffering from skin arterial conditions such as cutaneous vasculitis. Cutaneous vasculitis can cause elastic lamina damage in the small arteries in the skin, and use of the materials for delivery of treatment compositions can alleviate such damage.

Agents that can be delivered by use of the antibodies or antigen binding fragments thereof can include biologically active agents such as, and without limitation to, anticoagulants, antiplatelet agents, anti-inflammatory agents, SMC proliferation inhibitors, MMP and cathepsin inhibitors, cytostatic agents, antioxidants, chelating agents, elastin-stabilizing and regeneration agents, cytokines, enzymes, chemokines, radioisotopes, enzymatically active toxins, or chemotherapeutic agents.

In one embodiment, the materials can be utilized in delivery of genetic material that can include DNA and/or RNA nucleic acid constructs. Genetic material that can be delivered by use of the targeting materials described can include, without limitation, microRNA, transfer RNA, ribosomal RNA, silencing RNA, regulating RNA, antisense RNA, RNA interference, non-coding and coding RNA, DNA fragments, plasmids including genes in conjunction with regulatory sequences, precursors of functional constructs (e.g., mRNA precursors), DNA/RNA probes, etc., and the like.

An antibody or antigen binding fragment thereof can be utilized in delivery of one or more immunomodulatory agents that may increase or decrease production of one or more cytokines, up-or down-regulate self-antigen presentation, mask MHC antigens, or promote the proliferation, differentiation, migration, or activation state of one or more types of immune cells. Immunomodulatory agents include, but are not limited to, non-steroidal anti-inflammatory drugs (NSAIDs); topical steroids; cytokine, chemokine, or receptor antagonists; heterologous anti-lymphocyte globulin; etc.

In one embodiment a biologically active compound for targeted delivery can include a compound as may be utilized to directly treat degraded elastin. Such compounds can include those that can encourage crosslinking of elastin, so as to provide additional structural support to the connective tissue, and compounds that can upregulate elastin formation, particularly through increased formation and/or crosslinking of tropoelastin. For instance, an elastin crosslinking agent such as pentagalloylglucose (PGG) can be delivered by use of the antibodies or antigen binding fragments thereof. Biologically active compounds that can encourage the formation and/or crosslinking of tropoelastin so as to encourage formation of new elastic fibers include lysyl oxidase enzyme and/or agents that increase lysyl oxidase activity such as copper ions, or forskolin, which is a cyclic AMP (CAMP) inducer. Another compound that can be utilized to encourage crosslinking of tropoelastin is TGF-β, which has been shown to increase lysyl oxidase activity. Copper ions (Cu²⁺) can enhance extracellular transport of endogenous lysyl oxidase and functional activity of endogenous and exogenous lysyl oxidase by enabling electron transfer from oxygen to facilitate oxidative deamination and aldehyde formation at lysine residues in elastin. Accordingly, an antibody or antigen binding fragment thereof can be directly or indirectly linked with copper ions for delivery to a degraded elastic fiber.

In one embodiment, an agent that can dissolve minerals, such as for example, ethylenediaminetetraacetic acid (EDTA), which has been shown to be a versatile chelating agent; ethylene glycol-bis(β-aminoethyl ether)-N,N,N′,N′-tetraacetic acid (EGTA), a calcium specific chelator; ethylene glycol tetraacetic acid; nitrilotriacetic acid, hydroxyethyl ethylenediaminetriacetic acid; 8-Hydroxy-7-iodo-5-quinolinesulfonic acid; poly(gamma-glutamic acid; sodium thiosulphate; alpha-lipoic acid; bisphosphonates; diethylenetriaminepentaacetic acid (DTPA); and/or other chelators as are known in the art can be delivered.

An antibody or antigen binding fragment thereof can be directly or indirectly linked to an imaging agent. Upon binding to degraded elastic fiber via the antibody, an imaging agent can be used in determination of the location and extent of elastic fiber degradation and diagnosis of a related or unrelated disease condition. Imaging agents can include those for CT or MRI scans, or SPECT imaging as is known in the art. Detectable markers as may be directly or indirectly linked to the materials can include photoactivatable agents, fluorophores, radioisotopes, bioluminescent proteins or peptides, fluorescent tags (e.g., fluorescein, isothiocyanate (FITC), a cyanine dye, etc.), fluorescent proteins or peptides, affinity labels (e.g., biotin, avidin, protein A, etc.), enzymatic labels (e.g., horseradish peroxidase or alkaline phosphatase), or isotopic labels (e.g., 125I), gold particles, rods, x-ray opaque substances, and micro bubbles (e.g., for ultrasound imaging), or any other such detectable moiety to allow for detection of the antibody and optionally imaging of the area.

Further, while the above disclosure is related specifically to the delivery of nanoparticles to damaged elastin, the carrier, or nanoparticle, may be conjugated to a variety of antibodies. For example, antibodies may be ones that target neural tissues, connective tissues, or fatty tissues.

While the present method may be used to reduce calcium deposit within calcified blood vessels, the present method may also be used to treat blood vessels or other tissues which have not undergone calcification. As described above, the present inventors have found that cell senescence can occur before calcification occurs, particularly with respect to the medial artery calcification. In addition to this, the present inventors have found that administration of chelating agents reduces the number of cells which transition to senescence, even before calcium deposits are formed.

In addition to the conditions described above, such as those stemming from calcification, the nanoparticles of the present disclosure may be used as a treatment, or prophylactic, for a variety of other diseases. For instance, nanoparticles of the present disclosure may be used in the treatment of age-related diseases, such as neurodegeneration, neuropathy or age-related macular degeneration. Further, nanoparticles of the present disclosure may be used to treat conditions such as primary mitochondrial disease, glycogen storage disease, hereditary hemolytic anemia or hemolytic uremic syndrome.

In general, as will be discussed with respect to the examples below, the present disclosure may enable one of skill to treat a given condition based upon whether a gene should be upregulated or downregulated. Thus, the present disclosure also contemplates methods of upregulating or downregulating gene expression, such as those genes disclosed in Table 1.

The present disclosure may be used to treat a patient with any of the above described conditions, or as a preventative therapy. For instance, the treatment or therapy may be prescribed according to a dosing regimen, such as once daily for a week, for a period of weeks greater than or equal to 1 week, such as greater than 1 month, such as greater than 3 months, such as greater than 6 months, such as up to a year. Further, said dosing regimens may comprise multiple doses per day. In other embodiments of the present disclosure, the dosing regimen may be taken persistently, such as one dose, or multiple doses, per day. In other embodiments, doses may be taken weekly, or every other day.

Said doses are not particularly limited as to the concentration of the dose. A dose may comprise 2 mg/kg of body weight to 50 mg/kg of body weight, such as between 5 mg/kg of body weight to 30 mg/kg of body weight, such as between 10 mg/kg of body weight to 20 mg/kg of body weight, such as between 15 mg/kg of body weight to 18 mg/kg of body weight.

Administration of said doses are not particularly limited, but include intravenous administration or oral administration. In some embodiments, said doses may include stabilizers, pharmaceutical excipients, pH adjusters or antioxidants.

Administration methods of nanoparticle formulations of the present disclosure are not particularly limited. Without wishing to be bound to any particular mode of administration, nanoparticle formulations of the present disclosure may be administered using local administration techniques. For example, a catheter-based device may be used in order to inject and perfuse nanoparticle formulations locally to a tissue in need of treatment. The catheter-based device is not particularly limited, but may comprise a perforated balloon, or a double balloon which may isolate a portion of a tissue to be treated, such as an artery. In embodiments, the catheter-based device may comprise a device that can directly inject into an artery in need of treatment. In embodiments, the local delivery method may comprise use of a device which can administer nanoparticles periadventitially with or without radiologic guiding. In general, the present disclosure contemplates administration of nanoparticle formulations using local delivery techniques as are known in the art.

In embodiments, nanoparticle formulations of the present disclosure may be delivered using systemic administration. For instance, nanoparticle formulations may be delivered by injection of the nanoparticle formulation into a blood stream, wherein the nanoparticle formulation may be circulated throughout the body. While the present disclosure specifically contemplates methods such as intravenous administration, it is within the scope of the present disclosure to administer liposome formulations in other fashions, such as through an artery. In general, the present disclosure contemplates administration of nanoparticle formulations using systemic delivery techniques as are known in the art.

Additionally, delivery of the therapeutics, such as nanoparticles, of the present disclosure may be administered after patient evaluation. For instance, a clinician may evaluate a patient for a particular pathology and its resulting symptoms. Thereafter, administration of the therapeutic of the present disclosure may begin. Such an evaluation may be for any one or a combination of the conditions referenced above including, but not limited to, vascular calcification, macular degeneration, atherosclerosis or neurodegeneration. Further, a biopsy of a tissue may be taken from a patient, and thereafter evaluated for signs of disease, such as those mentioned previously. Further, the biopsied tissue may be evaluated for the accumulation of senescent cells or SASP. Thereafter, a therapeutic of the present disclosure may be administered to a patient.

The present invention may be better understood with reference to the examples, set forth below.

### EXAMPLES

While certain embodiments of the disclosed subject matter have been described using specific terms, such description is for illustrative purposes only, and it is to be understood that changes and variations may be made without departing from the spirit or scope of the subject matter.

### Nanoparticle Formation

The desolation method was used to make EDTA-loaded human serum albumin (HSA) nanoparticles following the method described earlier (Lei et al., 2014)] with slight modifications. Briefly, 200 mg of HSA (SeraCare, Milford, MA) was dissolved in 4 ml of deionized water, and 50 mg of EDTA (Fisher Scientific, NJ) was then dissolved in HSA solution. The pH of the solution was adjusted to 8.5. The solution was then added dropwise to the absolute ethanol (Sigma, St. Louis, MO) (1 mL/min) under constant stirring, followed by the addition of 25 μl of 8% glutaraldehyde as a crosslinker. The solution was incubated at room temperature for two hours with constant stirring at 800 rpm. Nanoparticles thus formed were centrifuged at 6000 rpm for 10 minutes, rinsed in deionized water (saturated with EDTA), and resuspended in phosphate-buffered saline before conjugating with thiolated anti-elastin antibody conjugation [REF]. 10 mg of formulated nanoparticles were PEGylated with 2.5 mg of α-maleimide-ω-N-hydroxysuccinimide ester poly (ethylene glycol) (mPEGNHS, MW 2000, Nanocs, NY, USA) for an hour at room temperature under gentle agitation. Meanwhile, 20 μg of custom-made humanized anti-elastin antibody was added to 68 μg of Traut's reagent (G-Biosciences, Saint Louis, MO) for antibody thiolation, and subsequently the mixture was incubated in 4-(2-hydroxyethyl)-1-piperazineethanesulfonic acid (HEPES) buffer (20 mM, pH=8.8) at room temperature for an hour under gentle agitation. Finally, thiolated antibody was added to the PEGylated nanoparticles and incubated overnight (16 hours) at 4° C. under gentle rocking for conjugation.

### Rat CKD Studies

The study was conducted in two parts. In the first part of the study, a short-duration in-vitro aortic ring culture model and an early-stage in-vivo chronic kidney disease model was employed to investigate whether senescence precedes calcification in the aorta. In both the early-stage models; in-vivo CKD model with adenine diet only and short-duration in-vitro ring culture; priming conditions for arterial calcification in the tunica media was simulated, and the samples were harvested before any signs of arterial calcification. The samples were tested for the presence of senescent cells and SASP markers, NLRP3, and Pit-1 (Phosphorus and calcium ion transporter) receptor expression in the aorta. After establishing the existence of senescent cells in the early stages of the disease, the second part commenced, which was to test whether senescence markers can be brought down by manipulating mineral imbalance in the tissue microenvironment. For this, targeted EDTA chelation therapy was used. Human serum albumin nanoparticles loaded with EDTA (EDTA NP) were conjugated with Flexibzumab antibody that can specifically target damaged elastin in the artery (Lei et al., 2014; Nosoudi et al., 2015; Sinha et al., 2014). The EDTA-NPs were injected iv in the late-stage CKD animals, and aortas from the animals were harvested to analyze calcification status, senescence phenotype, and NLRP3 expression. Overall, this study, using the in-vivo CKD model for the first time, provides evidence that senescent cell accumulation in the aorta precedes aorta calcification and emphasizes the role of mineral imbalance in senescence-associated secretory phenotype machinery in vascular smooth muscle cells.

Male Sprague Dawley rats age (12 weeks) were used for the study. Animals obtained from Charles River Laboratories were acclimatized for two weeks before starting the study and were maintained on a standard rodent diet (Teklad Global 18% Protein Rodent Diet, Madison, WI). During the entire study, the animals were monitored for body weight, temperature, and regular activities by an experienced veterinarian and were euthanized by saline perfusion under isoflurane anesthesia when they reached a humane endpoint of >20% weight loss.

The animals were randomly divided into treatment and control groups. Control group animals were maintained on a standard chow diet till the completion of the study, and the animals in the treatment groups were maintained on 0.75% Adenine diet containing 2.5% protein (TD. 130127, Madison, WI) with a modified schedule for 28 days as described previously. Twenty-four hours after the completion of the study, the animals were euthanized by saline perfusion under isoflurane anesthesia, and the aortas (from the heart to the iliac bifurcation), as well as other organs, including lungs, liver, kidneys, and spleen, were harvested and preserved accordingly for further analysis. Blood was also collected by cardiac puncture for serum analysis.

The animals were randomly divided into treatment and control groups. Control group animals were maintained on a standard chow diet till the completion of the study, and the animals in the treatment groups were maintained on a 0.75% Adenine diet containing 2.5% protein (TD. 130127, Madison, WI) with a modified schedule for 28 days as described previously. Five days after completion of the adenine diet, the animals in the treatment groups were either injected intraperitoneally with vitamin D3 (VitD3, Cholecalciferol, Sigma, #C9756-5G) (8.75 mg/kg/day formulated in olive oil) or equivalent olive oil as a vehicle for four consecutive days. 3 days post I.P injections the animals in the treatment group that received vitamin D injections were further randomly subdivided into two groups: (i) the EDTA NP group—in which the animals received two weekly injections (a total of 5 injections) of EDTA-HSA-EI-Ab nanoparticles (10 mg/kg IV), and (ii) the blank NP group—in which the animals received blank-HSA-EI-Ab nanoparticles with the same dosage as EDTA nanoparticles. After three weeks of nanoparticle therapy, the aortas (from the heart to the iliac bifurcation) and other organs, including lungs, liver, kidneys, and spleen, were harvested and preserved accordingly for further analysis. Blood was also collected for serum isolation and biomarker analysis.

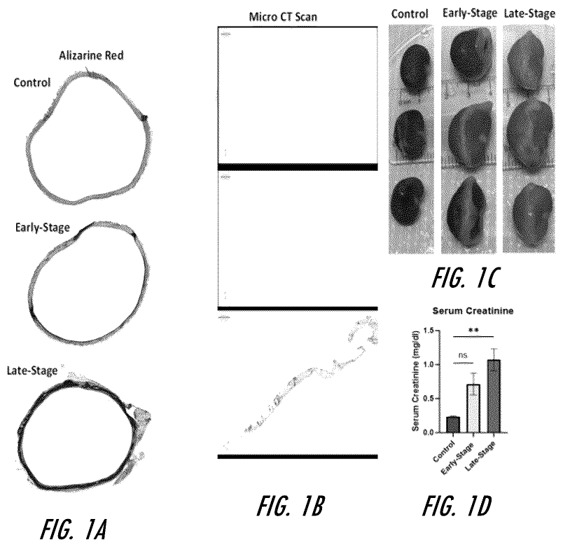

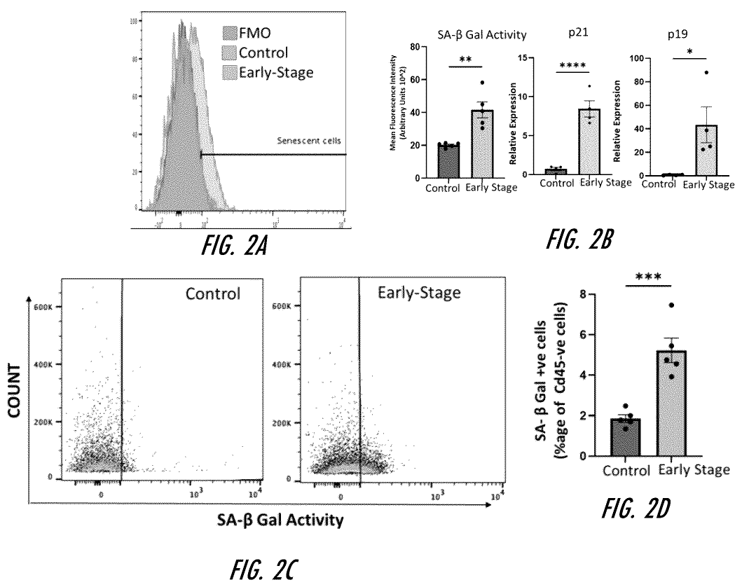

Calcium deposition (Alizarine red staining, Micro CT scans), gross kidney morphology, and serum creatinine levels in the aortas and serum of early and late-stage CKD animals was compared. Alizarin red and Micro CT scans revealed increased calcium deposition in the aortas of late-stage CKD animals, whereas negligible calcium accumulation was observed in the early-stage samples (FIG. 1A, FIG. 1B). Likewise, progressive increase in kidney size and serum creatinine levels was observed, evidence of progressive disease pathology (FIGS. 1C, 1D).

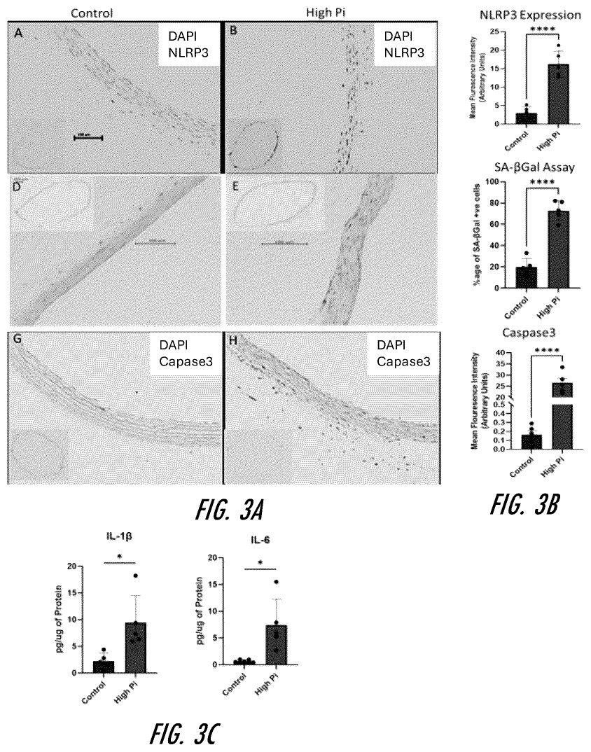

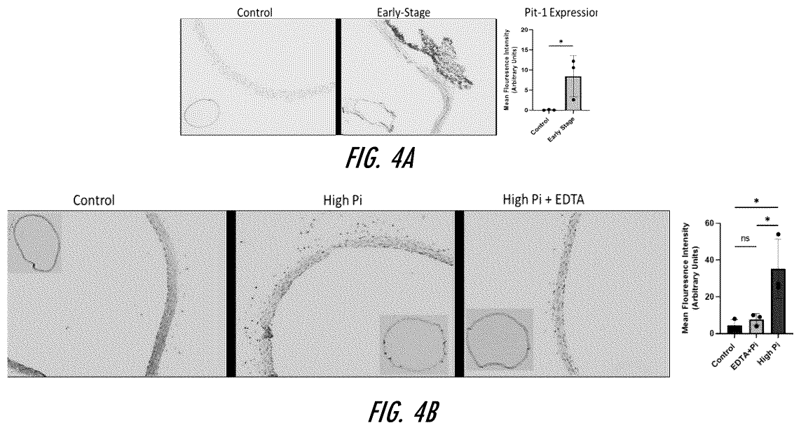

Primary cells isolated from abdominal aorta harvested from early-stage CKD animals (12 weeks old rats maintained on an adenine diet for four weeks) and respective controls (age-matched rats maintained on a chow diet) were subjected to flow cytometry for analysis of senescence markers (SA-β Gal activity, expression analysis of p19, p21). Upon comparison with age-matched control samples, a significant increase in SA-βGal activity, an increased expression of p19 and p21 genes in the aortas of early-stage CKD rats was observed, indicative of senescent cell accumulation (FIG. 2). Specifically, FIGS. 2A-2B show that early-stage CKD rats had an increased degree of activity of SA-βGal. FIG. 2B shows that early-stage CKD rats had increased expression of p21 and p19. FIG. 3C shows the count of senescent cells in the rat's aorta, and FIG. 2D shows the percent of senescent cells in the rat's aorta.

A short-duration in-vitro ring culture under a high phosphate medium (5 days), which simulates the early-stage CKD model, was used to test whether NLRP3 activation accompanies the presence of senescent cells. The aortic rings cultured did not show calcium deposition in the aorta. However, a significant increase in the percentage of senescent cells (as is evident by SA-βGal staining) and in the NLRP3 expression (as validated by IHC) in the high Pi group in comparison to the control group (FIGS. 3A-3B) was observed. Increased caspase3 expression was also observed in the aorta sections cultured under high phosphate conditions. ELISA from the culture supernatant revealed significant differences in the levels of IL-6 and IL-1β (FIG. 3C). These findings indicate that an imbalance in phosphate ion concentration, whether in the microenvironment or globally, can induce activation of NLRP3 signaling machinery and cellular senescence prior to calcification.

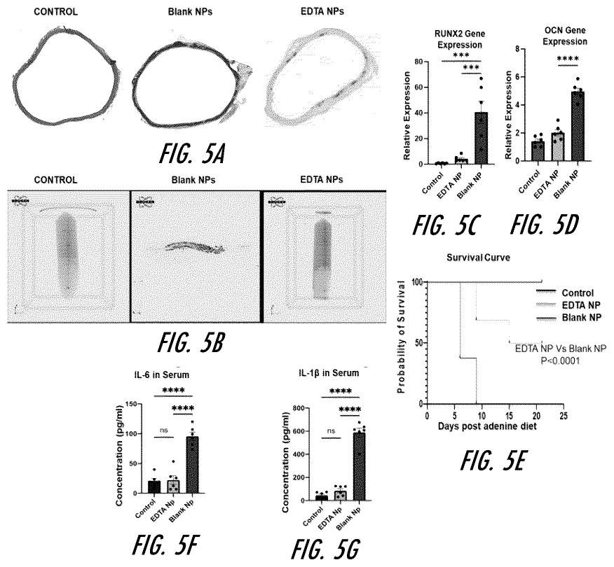

Aortic rings were harvested from age-matched healthy rats and cultured them for five days under normal or high Pi conditions with or without EDTA treatment (FIG. 4A). After the treatment duration, the aortas were analyzed for PiT-1 expression by IHC. A significant increase in Pit 1 expression was observed in the aorta cultured in High Pi conditions, which was brought down by EDTA treatment. It was also found that Pit-1 expression was upregulated in the aortas of early-stage CKD rats (FIG. 4B).

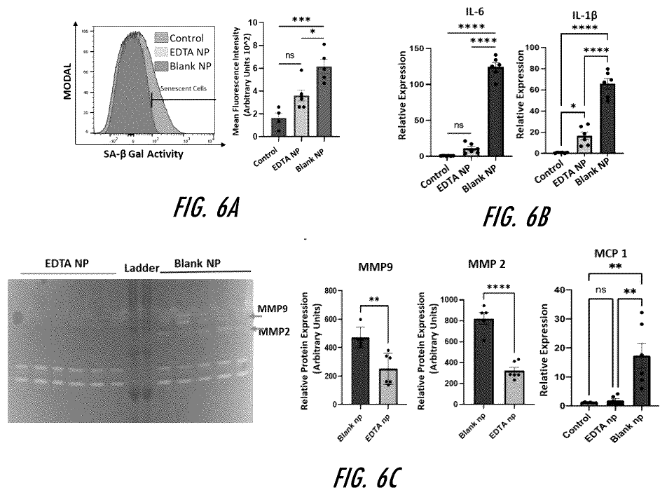

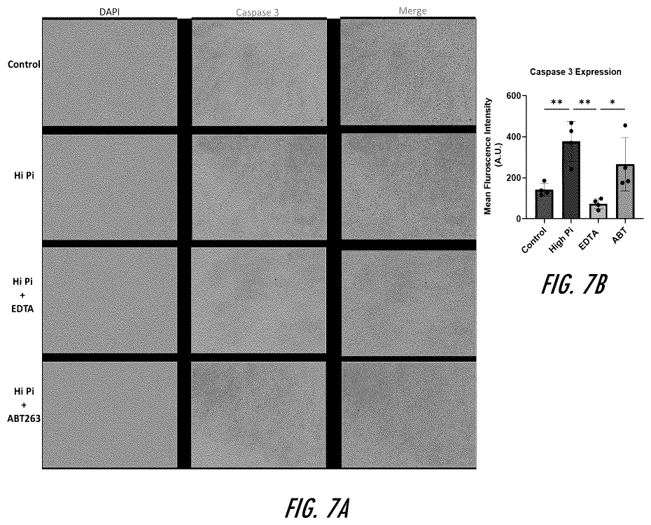

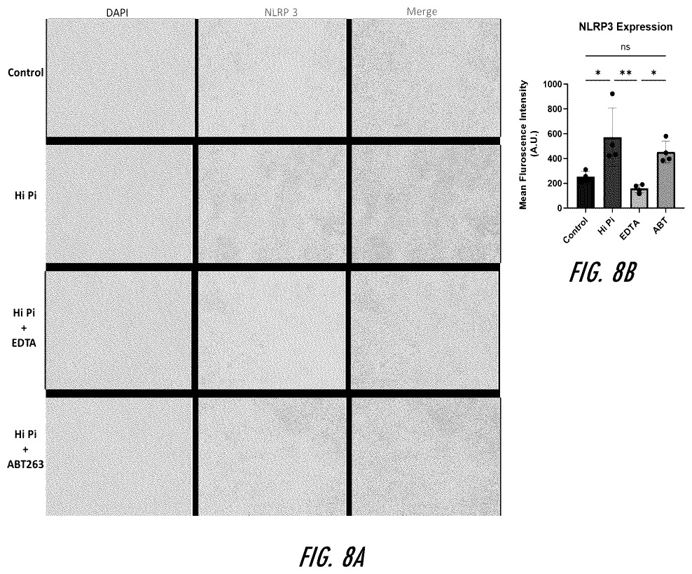

Further, whether EDTA NPs have a serotherapeutic effect was investigated. Using the CKD model, whether EDTA-NP therapy decreases calcium deposition in the aorta was validated. Calcium deposition in aorta were analyzed by Alizarin staining (FIG. 5A) and Micro CT scanning (FIG. 5B). Transcript level analysis of ossification markers Osteocalcin (OCN, FIG. 5D) and RUNX2 (FIG. 5C) and a decrease in their expression level revealed a decreased tendency towards osteoblastic phenotypic switching. Further a significant decrease in circulating levels of pro inflammatory cytokine IL-6 (FIG. 5F) and IL-1β (FIG. 5G) with a simultaneous significant improvement in the survival rate as is reflected by the Survival curve (FIG. 5E) was observed. The survival curve shows that the control remained at 100, the EDTA NP group fell to 50 after 20 days, and the blank NP fell to 0 before 10 days.

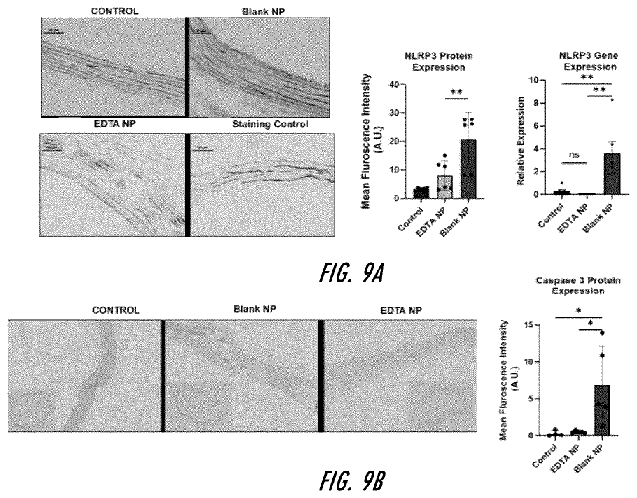

Treatment with EDTA nanoparticles also decreased the concentration of inflammatory cytokines, IL-6 and IL-1β in the serum and improved survival rate in the CKD rodent model (FIG. 6A). The transcriptional expression and activity of senescence and SASP markers (SA-β Gal, IL-6, IL-1β, BMP2, MCP1, MMP9, &2) in the aorta harvested from EL-EDTA-NPs, Blank NP (Flexibzumab conjugated Albumin nanoparticles that are not loaded with any drug, EL-BL-NPs) and control treatment group animals were compared (FIGS. 6A-6C). A significant decrease in senescence build-up and SASP markers in the aorta harvested from the EL-EDTA-NP treatment group (FIG. 6C) was observed.

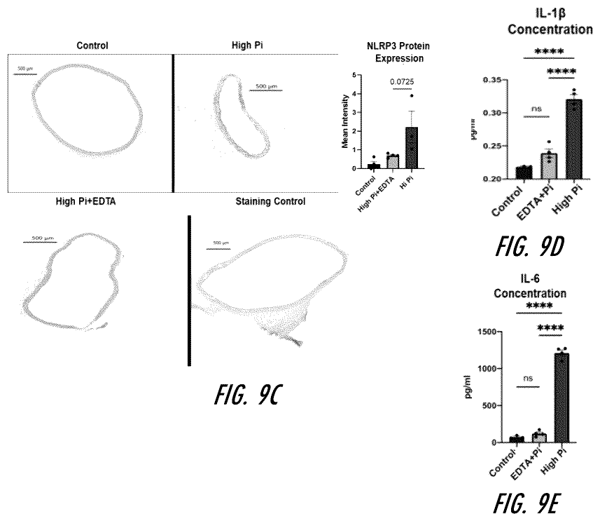

To examine whether EDTA induced apoptosis of senescent cells, a well established senolytic agent ABT 263, which is known to induce apoptosis by recruiting NLRP3 via caspase3 activation and tested it similarly to EDTA was used. EDTA treatment instead decreased Caspase 3 expression in immortalized human vascular smooth muscle cells exposed to high phosphate ion concentration, whereas ABT 263 treatment increased caspase-3 expression, indicating that EDTA does not induce NLRP3 mediated apoptosis was observed. Furthermore, a decrease in Caspase 3 expression suggests transcriptional inhibition of NLRP3 expression during the priming stage (FIGS. 7A-8B).

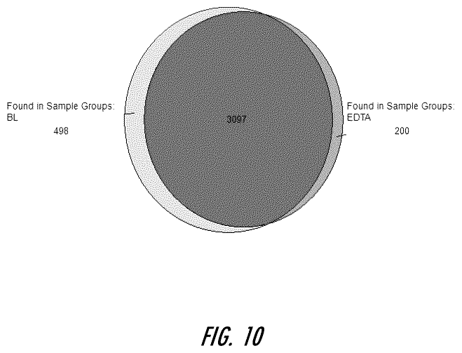

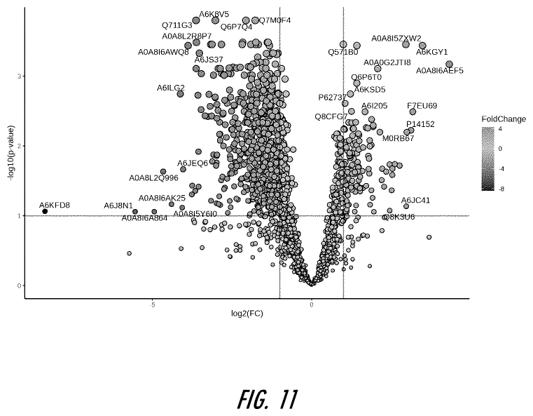

The expression of NLRP3 (FIG. 9A) and Caspase3 (FIG. 9B) in the calcified aorta harvested from late-stage CKD rats was evaluated as well as in the long-duration aortic ring culture model. In the calcified aortas harvested from both aortic ring culture and CKD model, it was observed that treatment with EDTA and EDTA NPs, respectively, caused a significant decrease in NLRP3 expression as quantified using IHC, and qPCR as well as a decrease in the concentration of IL-1 β and IL-6 in the culture supernatants (FIGS. 9C-9E).

### Proteomics

A 3-cm section of abdominal aorta was homogenized, and protein was isolated using T-Perm protein extraction buffer as per manufacturer protocol (Thermo Fischer). Protein concentrations were determined using BCA assay kit (Thermo Fisher Scientific, Watham, MA, USA). Protein samples were normalized to 60 μg with MS-grade water and proteins were reduced with 20 mM tris (2-carboxyethyl) phosphine (TCEP) by incubating at 50° C. for 15 minutes. Proteins were brought to room temperature and then alkylated with 40 mM iodoacetamide (IAA) by incubating in dark at room temperature for 30 min. Tryptic digestion was performed using suspension traps (S-trap mini, Protifi, Fairport, NY, USA) following the manufacturer's protocol. The reduced and alkylated proteins (60 μg) were acidified with 10:1 sample/12% phosphoric acid (v/v) and then diluted with 1:7 acidified sample/Binding Buffer (v/v). Proteins were loaded to S-traps in aliquots of 200 μL, centrifuged at 4,000 g for 30 sec, discarding the flow-through, washed six times with 200 μL Binding Buffer, discarding the flow-through, and centrifuged at 4,000 g for 1 min. Trypsin protease was added 1:10 trypsin/sample protein (w/w), centrifuged 1000 rpm for 10 sec, and incubated in dark water bath at 37° C. for 13 hours. Peptides were eluted from S-traps with 50 mM ammonium bicarbonate in water, centrifuged at 3,000 rpm for 1 min, repeated elution with 0.1% formic acid in water, and then with 40% acetonitrile containing 0.1% formic acid in water, combining eluates in one 2 mL tube. Peptides were concentrated by evaporation under nitrogen gas stream and reconstituted to a final protein concentration of 1.2 μg/μL in 95% water, 5% acetonitrile, 0.1% formic acid containing 50 nM diluted Pierce™ Peptide Retention Time Calibration Mixture.

Protein digests were analyzed on an UltiMate™ 3000 UHPLC (Thermo Scientific) coupled to an Orbitrap Fusion™ Tribrid mass spectrometer (Thermo Scientific) equipped with EASY-spray™ nano-flow source. Two microgram protein digests in 1 μL injections were loaded onto PepMap™ RSLC C18 NanoSpray column (2 μm, 100 Å, 75 μm×50 cm). Peptides were separated using a solvent gradient with 0.1% formic acid in water (mobile phase A) and 0.1% formic acid in 80% acetonitrile (mobile phase B) at a flow rate of 250 nl min⁻¹. For peptide separation, the column was initially equilibrated at 4% B for 3 min., increased to 30% B at 90 min, increased to 55% B at 120 min, increased to 90% B at 130 min, held at 90% B until 134 min., and then decreased to 4% B at 135 min. The solvent gradient included a column flush method that consisted of three rapid gradient flushes of 4% B to 90% B holding at each for 4min and then re-equilibrated at 4% for 25 min. Peptides were ionized in positive ionization mode using 2.2kV spray voltage, 2 Arb sweep gas flow, and 275° C. ion transfer tube temperature. The MS¹ scan (m/z 300-1,500) was performed in orbitrap mass analyzer at 500,000 resolution with a cycle time of 2 sec. MS² scans were collected for ions that passed the following filters: peptide monoisotopic peak determination, charge states 2-7, dynamic exclusion duration of 40 seconds for 10 ppm mass tolerance, minimum intensity of 1.9E4, and isotope exclusion. MS² scans were acquired in the ion trap mass analyzer with an isolation window of 1.2 amu, following activation with collision-induced dissocation (CID) of 35% energy. Data were processed in Proteome Discoverer (Version 3.1.0.638, Thermo Fisher Scientific) with FDR confidence <0.01, using FASTA files for Ratus norvegicus: Rattus norvegicus (sp_tr_incl_isoforms TaxID=10116_and_subtaxonomies), downloaded October 20; 83181 sequences. Normalized data was used from Proteome discoverer for analysis. Features with >50% missing values were removed, for the remaining missing values were estimated with BPCA. The data was auto scaled.

An untargeted top-down proteomics analysis of total protein isolated from abdominal aorta of EDTA NP treated Vs Blank NPs treated animals was performed (results shown in Table 1). The analysis revealed that 498 proteins were exclusively expressed in the Blank treatment group and 200 proteins were exclusively expressed in the EDTA treatment group (FIG. 10).

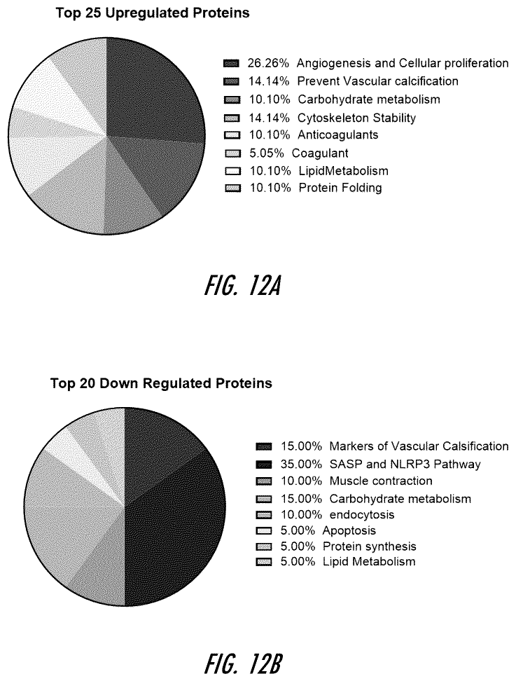

A total of 824 proteins were identified that were significantly differentially expressed when under treatment with the EDTA nanoparticles as compared to the blank nanoparticles. (FIG. 11). Amongst the top 25 upregulated proteins, 26% are directly influence cell proliferation and angiogenesis (Anaxa8, Enpep, Fgf8b, Clec11a, Crlf1, Agc1, Lancl2), 14% prevent osteoblastic transition of VSMCs (MGP, Phosphorylated form of Spp1, and ANKH), 14% play a role in cytoskeleton stabilization (RCG23467, Dmd, and Actb), 10% are involved in mediating proper protein folding (Ppidl1, Hmga1), 10% are involved in anabolic Lipid and carbohydrate metabolism (Mdh1 Mor2, Pla2g4a), and another 10 and 5% are anticoagulant and coagulants (FIG. 12A).

Amongst the top 20 downregulated proteins, 35% were directly involved in facilitating SASP processes (Uchl1, H1-1 Hist1h1a, HP, Cfb C2, Tf, Serpina1), 15% were markers of vascular calcification (Ckmt2, Fetub, Pvalb Pva), 10% regulate muscle contraction (Myl2, RCG36716), 5% are involved in each Lipid metabolism, protein synthesis and apoptosis (Iah1 Harpb64, Eef1a2 Kcnq2, Kng1) (FIG. 12B). The list of downregulated proteins that regulate NLRP3 activation was screened. PIT-1, Csnk2b, and Rbp4 are known to regulate priming of the NLRP3 pathway via the Jak2/MAPK/PI3K-Akt signaling axis. Proteins such as S100a9 and Tf are involved in phase 2 activation through ROS and ion flux-mediated signaling while Jpt1, Sumo2, and Pycard contribute to the assembly and stabilization of the NLRP3 inflammasome complex. Additional downregulated proteins such as Smap1, Serpina3k, Serpin2b, Bcl2l13, and Bin1 serve as molecular markers of senescence and chronic inflammation.

TABLE 1
List of genes downregulated and upregulated by EDTA NP therapy.

| | | | | Abs. | | |
|---|---|---|---|---|---|---|
| | | | | Val. | | |
| Gene | | | | Log | Accesion | |
| Reference | FC | Log2(FC) | P-Value | P-value | Number | Gene Symbol |
| A6KFD8 | 0.003011 | −8.3757 | 0.086559 | 1.0627 | A6KFD8 | Myl1 |
| A6J8N1 | 0.02143 | −5.5442 | 0.087337 | 1.0588 | A6J8N1 | Ckm |
| A0A8I6A864 | 0.032513 | −4.9428 | 0.087426 | 1.0584 | A0A8I6A864 | Ckmt2 |
| A0A8L2Q996 | 0.039492 | −4.6623 | 0.023298 | 1.6327 | A0A8L2Q996 | Eef1a2 |
| A0A8I6AK25 | 0.047357 | −4.4003 | 0.068295 | 1.1656 | A0A8I6AK25 | Eno3 |
| A6ILG2 | 0.057362 | −4.1238 | 0.001801 | 2.7445 | A6ILG2 | Necap1 |
| A0A8I5Y610 | 0.059676 | −4.0667 | 0.076524 | 1.1162 | A0A8I5Y610 | Myl2 |
| A6JEQ6 | 0.060991 | −4.0353 | 0.02163 | 1.6649 | A6JEQ6 | Serpina3c |
| A0A8I6AWQ8 | 0.067863 | −3.8812 | 0.000366 | 3.4371 | A0A8I6AWQ8 | Fetub |
| P02625 | 0.073666 | −3.7629 | 0.049557 | 1.3049 | P02625 | Pvalb |
| Q9R0P9 | 0.074293 | −3.7506 | 0.037038 | 1.4314 | Q9ROP9 | Uchl1 |
| P09117 | 0.079504 | −3.6528 | 0.044088 | 1.3557 | P09117 | Aldoc |
| Q711G3 | 0.08076 | −3.6302 | 0.000159 | 3.7985 | Q711G3 | Iah1 |
| A0A0G2JZ73 | 0.080919 | −3.6274 | 0.00078 | 3.1081 | A0A0G2JZ73 | Serpina1 |
| A0A8L2R8P7 | 0.081458 | −3.6178 | 0.000328 | 3.4835 | A0A8L2R8P7 | Kng1 |
| D4A3K5 | 0.084177 | −3.5704 | 0.001878 | 2.7263 | D4A3K5 | H1-1 |
| O35077 | 0.085198 | −3.553 | 0.012093 | 1.9175 | O35077 | Gpd1 |
| A0A8I5ZPF0 | 0.085421 | −3.5493 | 0.038376 | 1.4159 | A0A8I5ZPF0 | Hp |
| A6JS37 | 0.086837 | −3.5255 | 0.00047 | 3.3281 | A6JS37 | Kng2 |
| P02770 | 0.09014 | −3.4717 | 0.003022 | 2.5198 | P02770 | Alb |
| G3V615 | 0.090598 | −3.4644 | 0.000922 | 3.0352 | G3V615 | Cfb |
| A0A0H2UHJ1 | 0.098299 | −3.3467 | 0.003699 | 2.4319 | A0A0H2UHJ1 | S100a9 |
| A0A0G2JSH5 | 0.10493 | −3.2525 | 0.003793 | 2.4211 | A0A0G2JSH5 | Alb |
| A6HSL7 | 0.10711 | −3.2229 | 0.001825 | 2.7386 | A6HSL7 | rCG_60140 |
| A6ILD6 | 0.10981 | −3.1869 | 0.002714 | 2.5663 | A6ILD6 | rCG_29704 |
| Q7TMC7 | 0.11132 | −3.1672 | 0.000352 | 3.454 | Q7TMC7 | Tf |
| A0A8L2QB18 | 0.1119 | −3.1597 | 0.06091 | 1.2153 | A0A8L2QB18 | Pyroxd2 |
| F7FE81 | 0.11357 | −3.1384 | 0.003732 | 2.4281 | F7FE81 | Ndufa7 |
| A0A5H1ZRV3 | 0.11441 | −3.1277 | 0.000352 | 3.454 | A0A5H1ZRV3 | Tppp |
| P55053 | 0.11489 | −3.1216 | 0.016632 | 1.779 | P55053 | Fabp5 |
| A0A8I6AC90 | 0.11796 | −3.0836 | 0.013343 | 1.8747 | A0A8I6AC90 | Myom2 |
| F7FLZ6 | 0.11807 | −3.0823 | 0.00097 | 3.0132 | F7FLZ6 | Htra2 |
| A0A8L2UK05 | 0.1224 | −3.0303 | 0.001997 | 2.6995 | A0A8L2UK05 | Agt |
| A6IJL5 | 0.12257 | −3.0283 | 0.002034 | 2.6916 | A6IJL5 | Lap3 |
| A6K8V5 | 0.12309 | −3.0222 | 0.000159 | 3.7985 | A6K8V5 | Cfd |
| A0A8I6A6U5 | 0.12502 | −2.9998 | 0.019142 | 1.718 | A0A8I6A6U5 | Pcbd1 |
| Q9JI91 | 0.12594 | −2.9891 | 0.066199 | 1.1792 | Q9JI91 | Actn2 |
| A6K3D6 | 0.12605 | −2.9879 | 0.017419 | 1.759 | A6K3D6 | Phgdh |
| P04633 | 0.12653 | −2.9825 | 0.013841 | 1.8588 | P04633 | Ucp1 |
| O55006 | 0.12728 | −2.9739 | 0.002554 | 2.5927 | | |
| A0A0G2K8U8 | 0.12729 | −2.9738 | 0.004016 | 2.3962 | A0A0G2K8U8 | Fcgr3a |
| A0A8I6A9A6 | 0.1277 | −2.9692 | 0.013391 | 1.8732 | A0A8I6A9A6 | Chl1 |
| A6KKH1 | 0.13034 | −2.9396 | 0.003105 | 2.5079 | A6KKH1 | Gc |
| E0A3N4 | 0.13099 | −2.9325 | 0.00078 | 3.1081 | | |
| A0A8I5ZS45 | 0.13197 | −2.9217 | 0.067982 | 1.1676 | A0A8I5ZS45 | Ppp5c |
| G3V9S4 | 0.13376 | −2.9023 | 0.052665 | 1.2785 | G3V9S4 | Dbh |
| A0A096MKB7 | 0.13449 | −2.8944 | 0.006803 | 2.1673 | A0A096MKB7 | Csnk2b |
| A0A8I6GFV5 | 0.13502 | −2.8888 | 0.004585 | 2.3387 | A0A8I6GFV5 | Rbp4 |
| A0A0G2K1S6 | 0.13625 | −2.8757 | 0.006778 | 2.1689 | A0A0G2K1S6 | Me1 |
| P14046 | 0.13641 | −2.8739 | 0.008329 | 2.0794 | P14046 | A1i3 |
| A0A0G2K9Y5 | 0.13681 | −2.8698 | 0.00078 | 3.1081 | A0A0G2K9Y5 | Hrgl1 |
| A0A0G2JYM0 | 0.13691 | −2.8687 | 0.070302 | 1.153 | A0A0G2JYM0 | Ldb3 |
| D3ZN64 | 0.13964 | −2.8402 | 0.030984 | 1.5089 | D3ZN64 | Col28a1 |
| A6JPR7 | 0.14037 | −2.8327 | 0.000352 | 3.454 | A6JPR7 | Klkb1 |
| A0A8I6AAQ9 | 0.14099 | −2.8264 | 0.000917 | 3.0374 | A0A8I6AAQ9 | Cfi |
| A6K5K8 | 0.14365 | −2.7994 | 0.027607 | 1.559 | A6K5K8 | Ptgr3 |
| A6JCP0 | 0.14476 | −2.7883 | 0.003526 | 2.4527 | A6JCP0 | Fah |
| A6IRE9 | 0.14586 | −2.7774 | 0.003385 | 2.4705 | AGIRE9 | Dtx3 |
| A6IDW2 | 0.14668 | −2.7692 | 0.009998 | 2.0001 | A6IDW2 | Asns |
| F7EWL4 | 0.14784 | −2.7579 | 0.000959 | 3.0183 | F7EWL4 | Blk |
| D3ZV82 | 0.14852 | −2.7512 | 0.041591 | 1.381 | D3ZV82 | Gbp7 |
| Q6LE95 | 0.14911 | −2.7456 | 0.00097 | 3.0132 | | |
| A0A8I5ZTD4 | 0.15087 | −2.7286 | 0.002216 | 2.6544 | A0A8I5ZTD4 | Dbi |
| A6KFG3 | 0.15119 | −2.7255 | 0.039591 | 1.4024 | A6KFG3 | Atic |
| A0A8I6B1P6 | 0.15247 | −2.7134 | 0.004963 | 2.3043 | A0A8I6B1P6 | Clybl |
| A0A8I5ZMF1 | 0.15647 | −2.6761 | 0.007543 | 2.1225 | A0A8I5ZMF1 | Cmbl |
| B2RY15 | 0.1589 | −2.6538 | 0.002443 | 2.612 | B2RY15 | Tln2 |
| G3V733 | 0.15907 | −2.6523 | 0.029436 | 1.5311 | G3V733 | Syn2 |
| A6IR38 | 0.16125 | −2.6326 | 0.090953 | 1.0412 | A6IR38 | Gap43 |
| A0A9K3Y7W5 | 0.1627 | −2.6197 | 0.000918 | 3.0372 | A0A9K3Y7W5 | Gbp2 |
| P50115 | 0.16601 | −2.5906 | 0.000767 | 3.1154 | P50115 | S100a8 |
| A6ICE6 | 0.16634 | −2.5878 | 0.011276 | 1.9479 | A6ICE6 | Rnpep |
| A0A8I6GKQ5 | 0.1675 | −2.5778 | 0.001582 | 2.8009 | A0A8I6GKQ5 | Fkbp3 |
| A0A8I6GJH7 | 0.16823 | −2.5715 | 0.061432 | 1.2116 | A0A8I6GJH7 | Tstd3 |
| Q3MHC0 | 0.16912 | −2.5639 | 0.014056 | 1.8521 | Q3MHC0 | Jam2 |
| A0A8I6GHQ4 | 0.16926 | −2.5627 | 0.024948 | 1.603 | A0A8I6GHQ4 | Ephx2 |
| A0A8I6AHW7 | 0.17038 | −2.5532 | 0.015467 | 1.8106 | A0A8I6AHW7 | Smap1 |
| A6IKP7 | 0.17159 | −2.543 | 0.075447 | 1.1224 | A6IKP7 | Pgam2 |
| A0A0G2K014 | 0.17166 | −2.5424 | 0.002008 | 2.6973 | A0A0G2K014 | Lcp1 |
| A0A0H2UI21 | 0.17241 | −2.5361 | 0.000922 | 3.0352 | A0A0H2UI21 | Crat |
| A0A8L2PZQ2 | 0.17387 | −2.5239 | 0.019538 | 1.7091 | A0A8L2PZQ2 | S100b |
| A1A5L2 | 0.1746 | −2.5179 | 0.005806 | 2.2361 | A1A5L2 | Pgm1 |
| P13668 | 0.17493 | −2.5151 | 0.024883 | 1.6041 | P13668 | Stmn1 |
| A0A8I6A568 | 0.17501 | −2.5144 | 0.003177 | 2.4979 | A0A8I6A568 | Gphn |
| A6IUW5 | 0.17727 | −2.496 | 0.005806 | 2.2361 | A6IUW5 | Isg15 |
| A0A8I5ZUT0 | 0.17765 | −2.4929 | 0.023054 | 1.6373 | A0A8I5ZUT0 | Kyat3 |
| P02696 | 0.17923 | −2.4801 | 0.004585 | 2.3387 | P02696 | Rbp1 |
| G3V918 | 0.18105 | −2.4656 | 0.000746 | 3.1276 | G3V918 | Gart |
| A0A8I6AT16 | 0.18244 | −2.4545 | 0.010028 | 1.9988 | A0A8I6AT16 | Echdc1 |
| Q9QVC8 | 0.18307 | −2.4495 | 0.006931 | 2.1592 | Q9QVC8 | Fkbp4 |
| D3ZA93 | 0.18473 | −2.4365 | 0.002595 | 2.5859 | D3ZA93 | Acot13 |
| A2RRU1 | 0.18495 | −2.4348 | 0.037938 | 1.4209 | A2RRU1 | Gys1 |
| A0A8I6AUX5 | 0.18621 | −2.425 | 0.00931 | 2.031 | A0A8I6AUX5 | Tmsb4x |
| Q9R290 | 0.18631 | −2.4242 | 0.002657 | 2.5755 | Q9R290 | Fabp4 |
| A0A0G2K896 | 0.18771 | −2.4134 | 0.02371 | 1.6251 | A0A0G2K896 | Inhca |
| A618H4 | 0.18898 | −2.4037 | 0.003946 | 2.4038 | A618H4 | Coq7 |
| P04905 | 0.18907 | −2.403 | 0.004986 | 2.3023 | P04905 | Gstm1 |
| A0A8I6A6P6 | 0.1895 | −2.3997 | 0.004734 | 2.3248 | A0A8I6A6P6 | Csad |
| A0A8I5Y697 | 0.19005 | −2.3956 | 0.01147 | 1.9404 | A0A8I5Y697 | Ap1g1 |
| Q5PPN5 | 0.19048 | −2.3923 | 0.020752 | 1.683 | Q5PPN5 | Tppp3 |
| A0A0G2JWX1 | 0.19119 | −2.3869 | 0.017024 | 1.7689 | A0A0G2JWX1 | Psmd11 |
| A0A8L2UHL4 | 0.1941 | −2.3651 | 0.056874 | 1.2451 | A0A8L2UHL4 | TagIn3 |
| D3ZUV3 | 0.19563 | −2.3538 | 0.000767 | 3.1154 | D3ZUV3 | Eif2a |
| A0A8I5XWT5 | 0.19571 | −2.3532 | 0.008346 | 2.0785 | A0A8I5XWT5 | Sirt3 |
| P20761 | 0.19573 | −2.3531 | 0.017479 | 1.7575 | P20761 | Igh-1a |
| B1H271 | 0.19641 | −2.3481 | 0.019362 | 1.713 | B1H271 | Slc25a42 |
| Q5M7V3 | 0.19878 | −2.3307 | 0.005095 | 2.2929 | Q5M7V3 | LOC367586 |
| A0A8I5Y712 | 0.20088 | −2.3156 | 0.058576 | 1.2323 | A0A8I5Y712 | Kctd12 |
| A6KRZ1 | 0.20237 | −2.305 | 0.004792 | 2.3195 | A6KRZ1 | Yme1l1 |
| B2RSR7 | 0.20265 | −2.3029 | 0.002145 | 2.6685 | B2RSR7 | Gpd1l |
| A6HVF3 | 0.20417 | −2.2922 | 0.0118 | 1.9281 | A6HVF3 | Abcd3 |
| A0A0G2JXT3 | 0.2051 | −2.2856 | 0.006779 | 2.1689 | A0A0G2JXT3 | Fdps |
| A0A8I6AI41 | 0.20526 | −2.2845 | 0.005563 | 2.2547 | A0A8I6AI41 | ENSRNOG0 |
| | | | | | | 0000066406 |
| A0A8I6AID3 | 0.20848 | −2.262 | 0.019362 | 1.713 | A0A8I6AID3 | Igkvl-ps19 |
| A0A8I6AQ74 | 0.2087 | −2.2605 | 0.015782 | 1.8018 | A0A8I6AQ74 | ENSRNOG0 |
| | | | | | | 0000064086 |
| A6J957 | 0.21062 | −2.2473 | 0.013841 | 1.8588 | A6J957 | Lipe |
| A0A8I6APJ0 | 0.2112 | −2.2433 | 0.000749 | 3.1253 | A0A8I6APJ0 | Lsm4 |
| A0A8L2QC84 | 0.21158 | −2.2407 | 0.002347 | 2.6295 | A0A8L2QC84 | Cotl1 |
| A0A8L2QX10 | 0.21345 | −2.228 | 0.035335 | 1.4518 | A0A8L2QX10 | Glul |
| P05545 | 0.21454 | −2.2207 | 0.030262 | 1.5191 | P05545 | Serpina3k |
| F1LR92 | 0.21487 | −2.2184 | 0.012965 | 1.8872 | F1LR92 | Serpina3m |
| A0A8I5ZY29 | 0.21503 | −2.2174 | 0.001713 | 2.7662 | A0A8I5ZY29 | Usp14 |
| A6HUV7 | 0.2156 | −2.2136 | 0.002132 | 2.6712 | A6HUV7 | Gstm5 |
| P54645 | 0.21793 | −2.1981 | 0.029354 | 1.5323 | P54645 | Prkaa1 |
| A0A0G2JZH0 | 0.218 | −2.1976 | 0.011847 | 1.9264 | A0A0G2JZH0 | Cab39 |
| A0A8I6ABQ2 | 0.22029 | −2.1826 | 0.009302 | 2.0314 | A0A8I6ABQ2 | Bcl2113 |
| A0A8I6A3F2 | 0.22041 | −2.1817 | 0.006779 | 2.1689 | A0A8I6A3F2 | AABR070 |
| | | | | | | 34730.2 |
| A612S2 | 0.22054 | −2.1809 | 0.000352 | 3.454 | A612S2 | Abhd14b |
| M0RD03 | 0.22196 | −2.1716 | 0.017024 | 1.7689 | M0RD03 | Eif4g1 |
| P07872 | 0.22289 | −2.1656 | 0.000328 | 3.4835 | P07872 | Acox1 |
| A0A8I5XV96 | 0.22487 | −2.1528 | 0.001793 | 2.7464 | A0A8I5XV96 | Syce2 |
| A6KI43 | 0.22654 | −2.1422 | 0.002443 | 2.612 | A6KI43 | Gss |
| Q7TMB9 | 0.22858 | −2.1292 | 0.028841 | 1.54 | Q7TMB9 | Serpina3l |
| Q8VID1 | 0.2286 | −2.1291 | 0.046138 | 1.3359 | Q8VID1 | Dhrs4 |
| A6K5M3 | 0.22901 | −2.1265 | 0.018543 | 1.7318 | A6K5M3 | Timm21 |
| A0A8I6GJ11 | 0.23001 | −2.1202 | 0.003201 | 2.4948 | A0A8I6GJ11 | Acss2 |
| A0A8I5ZUG1 | 0.23003 | −2.1201 | 0.029017 | 1.5374 | A0A8I5ZUG1 | Gpd2 |
| A0A213BPS1 | 0.23027 | −2.1186 | 0.010979 | 1.9595 | A0A213BPS1 | Gmfb |
| D3ZWD6 | 0.23095 | −2.1143 | 0.00097 | 3.0132 | D3ZWD6 | C8a |
| M0RC66 | 0.23177 | −2.1093 | 0.002595 | 2.5859 | M0RC66 | Ak1 |
| A0A8I6ARZ8 | 0.23302 | −2.1015 | 0.002043 | 2.6897 | A0A8I6ARZ8 | Itsn1 |
| F1LMX1 | 0.23525 | −2.0877 | 0.004622 | 2.3352 | F1LMX1 | Bin1 |
| Q80TB8 | 0.23713 | −2.0762 | 0.03861 | 1.4133 | Q80TB8 | Vat11 |
| Q01149 | 0.23923 | −2.0635 | 0.007398 | 2.1309 | Q01149 | Col1a2 |
| Q6P7Q4 | 0.23941 | −2.0624 | 0.000159 | 3.7985 | Q6P7Q4 | Glo1 |
| A0A0G2K3T7 | 0.23978 | −2.0602 | 0.004585 | 2.3387 | A0A0G2K3T7 | Ndrg2 |
| Q63159 | 0.24116 | −2.052 | 0.027708 | 1.5574 | Q63159 | Coq3 |
| A6HS83 | 0.24193 | −2.0473 | 0.012752 | 1.8944 | A6HS83 | Cyc1 |
| A0A140TAE6 | 0.24195 | −2.0472 | 0.017087 | 1.7673 | A0A140TAE6 | Mecr |
| Q6PJ91 | 0.24363 | −2.0372 | 0.004119 | 2.3852 | Q6PJ91 | Gstm7 |
| F7FPV0 | 0.24407 | −2.0346 | 0.004576 | 2.3395 | F7FPV0 | Umps |
| A0A8I5Y4S4 | 0.24415 | −2.0342 | 0.043304 | 1.3635 | A0A8I5Y4S4 | Enah |
| P06801 | 0.24471 | −2.0308 | 0.007133 | 2.1467 | P06801 | Me1 |
| P27881 | 0.2448 | −2.0303 | 0.039533 | 1.403 | P27881 | Hk2 |
| Q6IMX3 | 0.24525 | −2.0277 | 0.008149 | 2.0889 | Q6IMX3 | Acads |
| A617J2 | 0.24529 | −2.0274 | 0.004119 | 2.3852 | A617J2 | Hpx |
| Q3V453 | 0.24702 | −2.0173 | 0.030052 | 1.5221 | Q3V453 | Ywhae |
| F1MAC0 | 0.24716 | −2.0165 | 0.012648 | 1.898 | F1MAC0 | Ifi47 |
| Q03626 | 0.24759 | −2.014 | 0.007336 | 2.1345 | Q03626 | Mug1 |
| A61IA8 | 0.24854 | −2.0084 | 0.006358 | 2.1967 | A61IA8 | Decr1 |
| Q64213 | 0.24903 | −2.0056 | 0.003564 | 2.4481 | Q64213 | Sf1 |
| A613E3 | 0.24918 | −2.0048 | 0.004652 | 2.3324 | A613E3 | Ngp |
| A0A0A0MY22 | 0.24931 | −2.004 | 0.025746 | 1.5893 | A0A0A0MY22 | Siae |
| A0A8I6ALF6 | 0.25079 | −1.9954 | 0.061065 | 1.2142 | A0A8I6ALF6 | F11r |
| A0A8I5ZXI2 | 0.25212 | −1.9878 | 0.000733 | 3.1351 | A0A8I5ZXI2 | Arhgdib |
| A6ISH9 | 0.25264 | −1.9849 | 0.012682 | 1.8968 | A6ISH9 | Yars1 |
| Q8VCE0 | 0.25277 | −1.9841 | 0.029992 | 1.523 | Q8VCE0 | Atp1a3 |
| A6KG34 | 0.25288 | −1.9834 | 0.002846 | 2.5458 | A6KG34 | Tkt |
| A6KBW2 | 0.25374 | −1.9785 | 0.00353 | 2.4522 | A6KBW2 | Pld4 |
| Q3U6P5 | 0.25421 | −1.9759 | 0.061566 | 1.2107 | Q3U6P5 | Hnrnpc |
| A0A8L2PZ08 | 0.25462 | −1.9736 | 0.008696 | 2.0607 | A0A8L2PZ08 | Cox6a1 |
| Q5BJQ0 | 0.25483 | −1.9724 | 0.00791 | 2.1018 | Q5BJQ0 | Coq8a |
| F7F0H6 | 0.25719 | −1.9591 | 0.01209 | 1.9176 | F7F0H6 | RGD1309362 |
| P30713 | 0.25941 | −1.9467 | 0.003327 | 2.478 | P30713 | Gstt2 |
| M0R835 | 0.25966 | −1.9453 | 0.020889 | 1.6801 | M0R835 | Sf3b6 |
| Q9WVK7 | 0.2597 | −1.9451 | 0.002661 | 2.5749 | Q9WVK7 | Hadh |
| A0A8I6ABF6 | 0.26011 | −1.9428 | 0.004855 | 2.3138 | A0A8I6ABF6 | Suox |
| A0A0F7RQJ6 | 0.26016 | −1.9425 | 0.001386 | 2.8581 | A0A0F7RQJ6 | Ddt |
| A0A0G2K6X9 | 0.26016 | −1.9425 | 0.016626 | 1.7792 | A0A0G2K6X9 | Adk |
| A0A668KLB7 | 0.26096 | −1.9381 | 0.094455 | 1.0248 | A0A668KLB7 | Clic5 |
| A0A8I6ARU6 | 0.261 | −1.9379 | 0.014886 | 1.8272 | A0A8I6ARU6 | Galk1 |
| A6IMU4 | 0.26182 | −1.9334 | 0.008527 | 2.0692 | A6IMU4 | Ldhb |
| Q05144 | 0.26406 | −1.9211 | 0.000746 | 3.1276 | Q05144 | Rac2 |
| A0A0G2K1D2 | 0.26492 | −1.9164 | 0.000959 | 3.0183 | A0A0G2K1D2 | Rap1gds1 |
| P14141 | 0.26534 | −1.9141 | 0.015231 | 1.8173 | P14141 | Ca3 |
| A0A8I6AFL1 | 0.26623 | −1.9093 | 0.002714 | 2.5663 | A0A8I6AFL1 | ENSRNOG0 |
| | | | | | | 0000066785 |
| Q9CPU0 | 0.26663 | −1.9071 | 0.000352 | 3.454 | Q9CPU0 | Glo1 |
| Q5U2Q3 | 0.26675 | −1.9065 | 0.007336 | 2.1345 | | |
| A0A8L2QBL5 | 0.26706 | −1.9048 | 0.006362 | 2.1964 | A0A8L2QBL5 | Ttr |
| Q91Y78 | 0.26759 | −1.9019 | 0.01147 | 1.9404 | Q91Y78 | Uchl3 |
| A0A8I6A2H9 | 0.26827 | −1.8983 | 0.010739 | 1.969 | A0A8I6A2H9 | Twf2 |
| A0A0G2K899 | 0.26852 | −1.8969 | 0.001801 | 2.7445 | A0A0G2K899 | Acyp1 |
| A6K2U7 | 0.26968 | −1.8907 | 0.001103 | 2.9576 | A6K2U7 | Tmod4 |
| M0R5J4 | 0.27016 | −1.8881 | 0.002008 | 2.6973 | M0R5J4 | Eno1 |
| Q5FVT5 | 0.27057 | −1.8859 | 0.024526 | 1.6104 | Q5FVT5 | Pdk1 |
| B2GUZ6 | 0.27063 | −1.8856 | 0.007472 | 2.1266 | B2GUZ6 | Rtn4ip1 |
| A6KHG6 | 0.27079 | −1.8847 | 0.002657 | 2.5755 | A6KHG6 | Pygb |
| A0A8I6ADA8 | 0.27167 | −1.8801 | 0.029155 | 1.5353 | A0A8I6ADA8 | Abhd4 |
| A0A8I6AQI7 | 0.27218 | −1.8774 | 0.009372 | 2.0282 | A0A8I6AQI7 | Serpinf2 |
| A6KJ36 | 0.27247 | −1.8758 | 0.015231 | 1.8173 | A6KJ36 | rCG_50926 |
| B0BNM9 | 0.27288 | −1.8737 | 0.042838 | 1.3682 | B0BNM9 | GLTP |
| A6ID81 | 0.2731 | −1.8725 | 0.002216 | 2.6544 | A6ID81 | Prdx6 |
| Q8VI04 | 0.27343 | −1.8708 | 0.022631 | 1.6453 | Q8VI04 | Asrgl1 |
| A0A8I6AX97 | 0.27423 | −1.8665 | 0.00097 | 3.0132 | A0A8I6AX97 | Jpt1 |
| A8DUK2 | 0.27433 | −1.866 | 0.016611 | 1.7796 | A8DUK2 | Hbbt1 |
| P23457 | 0.27454 | −1.8649 | 0.006803 | 2.1673 | P23457 | Akr1c9 |
| A0A8I6ADK6 | 0.27552 | −1.8598 | 0.042429 | 1.3723 | A0A8I6ADK6 | Calb2 |
| A6HW34 | 0.27561 | −1.8593 | 0.004622 | 2.3352 | A6HW34 | Adh5 |
| O35303-5 | 0.27566 | −1.859 | 0.032222 | 1.4919 | | |
| A6K4X8 | 0.2759 | −1.8578 | 0.050281 | 1.2986 | A6K4X8 | Gbe1 |
| D3Z898 | 0.27597 | −1.8574 | 0.009721 | 2.0123 | D3Z898 | Samhd1 |
| Q6ZQ61 | 0.27829 | −1.8454 | 0.048579 | 1.3135 | Q6ZQ61 | Matr3 |
| A6KFS0 | 0.27834 | −1.8451 | 0.058572 | 1.2323 | A6KFS0 | Sncg |
| Q4G069 | 0.27849 | −1.8443 | 0.036125 | 1.4422 | Q4G069 | Rmdn1 |
| A0A8I5ZVT0 | 0.27897 | −1.8418 | 0.000675 | 3.1707 | A0A8I5ZVT0 | Tpt1 |
| A0A8I5ZUK2 | 0.2802 | −1.8355 | 0.054339 | 1.2649 | A0A8I5ZUK2 | Ubap21 |
| O08557 | 0.28088 | −1.832 | 0.010519 | 1.978 | O08557 | Ddah1 |
| G3V762 | 0.28196 | −1.8264 | 0.010489 | 1.9793 | G3V762 | Gfus |
| Q4VBH1 | 0.28243 | −1.824 | 0.012414 | 1.9061 | Q4VBH1 | lghg |
| F1M004 | 0.28342 | −1.819 | 0.026111 | 1.5832 | F1M004 | AABR070 |
| | | | | | | 40565.1 |
| A6KBW4 | 0.28405 | −1.8158 | 0.008581 | 2.0665 | A6KBW4 | RGD1309696_ |
| | | | | | | predicted |
| A0A8I5ZY90 | 0.28408 | −1.8156 | 0.001582 | 2.8009 | A0A8I5ZY90 | Hmgcl |
| A6JK21 | 0.28428 | −1.8146 | 0.001801 | 2.7445 | A6JK21 | Pdxk |
| P97584 | 0.2843 | −1.8145 | 0.023811 | 1.6232 | P97584 | Ptgr1 |
| E9PSP1 | 0.28483 | −1.8118 | 0.030052 | 1.5221 | E9PSP1 | Pltp |
| P05480-1 | 0.2852 | −1.81 | 0.085664 | 1.0672 | | |
| A6JZ88 | 0.28589 | −1.8065 | 0.004619 | 2.3354 | A6JZ88 | Akr1a1 |
| A0A8I6A7Q1 | 0.28662 | −1.8028 | 0.003177 | 2.4979 | A0A8I6A7Q1 | Memo1 |
| Q68FT1 | 0.28759 | −1.7979 | 0.029992 | 1.523 | Q68FT1 | Coq9 |
| P47967 | 0.28846 | −1.7936 | 0.01758 | 1.755 | P47967 | Lgals5 |
| A6JZ44 | 0.28936 | −1.789 | 0.0112 | 1.9508 | A6JZ44 | Cyp4b1 |
| Q6P6V0 | 0.28955 | −1.7881 | 0.005392 | 2.2683 | Q6P6V0 | Gpi |
| D4AC65 | 0.29084 | −1.7817 | 0.004796 | 2.3191 | D4AC65 | Coa7 |
| Q62639 | 0.29087 | −1.7815 | 0.030797 | 1.5115 | Q62639 | Rheb |
| D4A5L9 | 0.29198 | −1.7761 | 0.002714 | 2.5663 | D4A5L9 | Cycsl2 |
| A0A8L2URF4 | 0.29197 | −1.7761 | 0.049316 | 1.307 | A0A8L2URF4 | Ubl4a |
| Q7M0F4 | 0.29333 | −1.7694 | 0.000159 | 3.7985 | | |
| A616G5 | 0.29387 | −1.7667 | 0.048619 | 1.3132 | A616G5 | Map6 |
| A6JT75 | 0.29455 | −1.7634 | 0.000352 | 3.454 | A6JT75 | C8g |
| A0A1S6GWG6 | 0.29587 | −1.757 | 0.005415 | 2.2664 | A0A1S6GWG6 | Atp6v1b2 |
| A0A8I6AHG0 | 0.29649 | −1.7539 | 0.003816 | 2.4184 | A0A8I6AHG0 | Tax1bp3 |
| B9EKL6 | 0.2969 | −1.7519 | 0.000328 | 3.4835 | B9EKL6 | Ptp4a1 |
| P56391 | 0.29697 | −1.7516 | 0.018574 | 1.7311 | P56391 | Cox6b1 |
| A619B9 | 0.29757 | −1.7487 | 0.012504 | 1.903 | A619B9 | Coro1a |
| Q5U2R8 | 0.29794 | −1.7469 | 0.013391 | 1.8732 | Q5U2R8 | Mnda |
| A6K3G6 | 0.29842 | −1.7446 | 0.031115 | 1.507 | A6K3G6 | Man1a2 |
| D4A7L6 | 0.29942 | −1.7397 | 0.004585 | 2.3387 | D4A7L6 | Rpia |
| D3ZW55 | 0.29948 | −1.7395 | 0.000647 | 3.1893 | D3ZW55 | Itpa |
| Q80SW1 | 0.30145 | −1.73 | 0.001825 | 2.7386 | Q80SW1 | Ahcyl1 |
| A6HFM1 | 0.30228 | −1.7261 | 0.036905 | 1.4329 | A6HFM1 | Slc25a35 |
| A0A8I5XVU7 | 0.30237 | −1.7256 | 0.010511 | 1.9783 | A0A8I5XVU7 | Otub1 |
| A6KGD5 | 0.30246 | −1.7252 | 0.056154 | 1.2506 | A6KGD5 | C7 |
| P07335 | 0.30255 | −1.7248 | 0.021229 | 1.6731 | P07335 | Ckb |
| Q499R7 | 0.30256 | −1.7247 | 0.003201 | 2.4948 | Q499R7 | Ppa1 |
| A6JXS7 | 0.30314 | −1.722 | 0.004348 | 2.3618 | A6JXS7 | Pfdn4 |
| A0A8I5ZZZ2 | 0.3032 | −1.7217 | 0.002219 | 2.6539 | A0A8I5ZZZ2 | Serpina4 |
| A0A8L2UIQ9 | 0.30338 | −1.7208 | 0.006168 | 2.2099 | A0A8L2UIQ9 | Esd |
| D4A3E2 | 0.30394 | −1.7181 | 0.004911 | 2.3089 | D4A3E2 | Npepl1 |
| Q510D1 | 0.30397 | −1.718 | 0.004585 | 2.3387 | Q510D1 | Glod4 |
| Q3UYQ4 | 0.30426 | −1.7166 | 0.002443 | 2.612 | Q3UYQ4 | Api5 |
| A0A8I6AAB9 | 0.30499 | −1.7132 | 0.005415 | 2.2664 | A0A8I6AAB9 | Ldha |
| A0A8I5ZYB8 | 0.30537 | −1.7114 | 0.050324 | 1.2982 | A0A8I5ZYB8 | Sh3glb2 |
| A0A8I6ATE4 | 0.30602 | −1.7083 | 0.072059 | 1.1423 | A0A8I6ATE4 | Nmt1 |
| F1LYU4 | 0.30766 | −1.7006 | 0.001232 | 2.9095 | F1LYU4 | ENSRNOG0 |
| | | | | | | 0000071026 |
| A6HGJ6 | 0.30817 | −1.6982 | 0.006259 | 2.2035 | A6HGJ6 | Aspa |
| A0A4E9FT70 | 0.30827 | −1.6977 | 0.044922 | 1.3475 | A0A4E9FT70 | IGHG3 |
| A0A0G2K1F2 | 0.30867 | −1.6959 | 0.015276 | 1.816 | A0A0G2K1F2 | Acacb |
| Q68FU7 | 0.30892 | −1.6947 | 0.019743 | 1.7046 | Q68FU7 | Coq6 |
| A6ISG8 | 0.30922 | −1.6933 | 0.000536 | 3.2712 | A6ISG8 | Ak2 |
| G3UXA6 | 0.30936 | −1.6927 | 0.022997 | 1.6383 | G3UXA6 | Ptbp3 |
| A6K7M6 | 0.30965 | −1.6913 | 0.060729 | 1.2166 | A6K7M6 | Cpt1b |
| Q91Z05 | 0.31076 | −1.6861 | 0.034864 | 1.4576 | Q91Z05 | Ighg |
| A0A8I6A1T4 | 0.31116 | −1.6843 | 0.073702 | 1.1325 | A0A8I6A1T4 | Eif4g2 |
| A0A8I5Y4W3 | 0.31296 | −1.676 | 0.003629 | 2.4403 | A0A8I5Y4W3 | Rab12 |
| A619Y0 | 0.31307 | −1.6755 | 0.025607 | 1.5916 | A619Y0 | Pycard |
| D3Z7U7 | 0.31328 | −1.6745 | 0.021229 | 1.6731 | D3Z7U7 | Ehd2 |
| A6IEL2 | 0.31329 | −1.6744 | 0.000352 | 3.454 | A6IEL2 | Akr1b1 |
| A6J288 | 0.31335 | −1.6742 | 0.035825 | 1.4458 | A6J288 | Hscb |
| M0RCN6 | 0.31422 | −1.6701 | 0.075625 | 1.1213 | M0RCN6 | Igkv2-112l2 |
| A0A8I5ZQN0 | 0.31508 | −1.6662 | 0.001386 | 2.8581 | A0A8I5ZQN0 | Pebp1 |
| Q9CPV4 | 0.31605 | −1.6618 | 0.089397 | 1.0487 | Q9CPV4 | Glod4 |
| Q4QR73 | 0.31675 | −1.6586 | 0.004622 | 2.3352 | Q4QR73 | Dnaja4 |
| A0A8I5ZWS4 | 0.3171 | −1.657 | 0.005789 | 2.2374 | A0A8I5ZWS4 | Hook3 |
| A0A8I5ZQ09 | 0.31731 | −1.656 | 0.012594 | 1.8998 | A0A8I5ZQ09 | ENSRNOG0 |
| | | | | | | 0000064930 |
| A0A8L2UIC7 | 0.3177 | −1.6543 | 0.012843 | 1.8913 | A0A8L2UIC7 | Necap2 |
| F1LZJ4 | 0.31897 | −1.6485 | 0.008346 | 2.0785 | F1LZJ4 | Hyi |
| P30835 | 0.32036 | −1.6423 | 0.004734 | 2.3248 | P30835 | Pfkl |
| A6J4P6 | 0.32159 | −1.6367 | 0.006843 | 2.1648 | A6J4P6 | Etfa |
| A6K1N2 | 0.32222 | −1.6339 | 0.015231 | 1.8173 | A6K1N2 | Fscn1 |
| Q9CWF2 | 0.32248 | −1.6327 | 0.083598 | 1.0778 | Q9CWF2 | Tubb2b |
| P26369 | 0.32311 | −1.6299 | 0.024494 | 1.6109 | P26369 | U2af2 |
| A6KQR3 | 0.3233 | −1.6291 | 0.003908 | 2.4081 | A6KQR3 | C3 |
| D4A9W3 | 0.32462 | −1.6232 | 0.022652 | 1.6449 | D4A9W3 | Dglucy |
| P02793 | 0.32527 | −1.6203 | 0.010289 | 1.9876 | P02793 | Ftl1 |
| A0A0G2JSH2 | 0.32571 | −1.6183 | 0.066267 | 1.1787 | A0A0G2JSH2 | Bdh1 |
| A0A8I6A9W1 | 0.32646 | −1.615 | 0.041372 | 1.3833 | A0A8I6A9W1 | ENSRNOG00 |
| | | | | | | 000065670 |
| P55314 | 0.32656 | −1.6146 | 0.000635 | 3.1973 | P55314 | C8b |
| A6KUH4 | 0.32709 | −1.6122 | 0.061269 | 1.2128 | A6KUH4 | rCG_47027 |
| A0A0G2JZS2 | 0.32824 | −1.6072 | 0.001878 | 2.7263 | A0A0G2JZS2 | Pabpc1 |
| Q499N5 | 0.32831 | −1.6069 | 0.013841 | 1.8588 | Q499N5 | Acsf2 |
| A0A8I5ZY73 | 0.32851 | −1.606 | 0.046991 | 1.328 | A0A8I5ZY73 | Cadm2 |
| D3ZBP4 | 0.32857 | −1.6057 | 0.009318 | 2.0307 | D3ZBP4 | Mical1 |
| A6JLB7 | 0.32896 | −1.604 | 0.00097 | 3.0132 | A6JLB7 | Sod1 |
| Q4G064 | 0.33005 | −1.5993 | 0.098093 | 1.0084 | Q4G064 | Coq5 |
| A0A387KC71 | 0.33021 | −1.5985 | 0.089923 | 1.0461 | A0A387KC71 | Akr1c15 |
| A6JTG5 | 0.33065 | −1.5966 | 0.020062 | 1.6976 | A6JTG5 | Agpat2 |
| A6IHD2 | 0.3317 | −1.592 | 0.011739 | 1.9304 | A6IHD2 | Cpa3 |
| A0A8L2QJE6 | 0.33344 | −1.5845 | 0.001713 | 2.7662 | A0A8L2QJE6 | Nit2 |
| Q5FVJ0 | 0.33389 | −1.5826 | 0.009721 | 2.0123 | Q5FVJ0 | Rufy3 |
| A0A8I5ZUX9 | 0.334 | −1.5821 | 0.071936 | 1.1431 | A0A8I5ZUX9 | Ces1f |
| A6JQC2 | 0.33401 | −1.582 | 0.033952 | 1.4691 | A6JQC2 | Rtn4 |
| A0A8I5ZV20 | 0.33417 | −1.5814 | 0.030538 | 1.5152 | A0A8I5ZV20 | Timm44 |
| A6IW96 | 0.33426 | −1.581 | 0.003353 | 2.4746 | A6IW96 | Defa5 |
| A0A8I6AQW7 | 0.33459 | −1.5795 | 0.003436 | 2.4639 | A0A8I6AQW7 | Msi2 |
| A0A1S6GWJ8 | 0.33524 | −1.5767 | 0.022867 | 1.6408 | A0A1S6GWJ8 | Hnrnpm |
| Q6PER3 | 0.33649 | −1.5714 | 0.008346 | 2.0785 | Q6PER3 | Mapre3 |
| G3V6U3 | 0.33692 | −1.5695 | 0.040533 | 1.3922 | G3V6U3 | Alg2 |
| A0A8I6G5T6 | 0.33737 | −1.5676 | 0.001029 | 2.9874 | A0A8I6G5T6 | Pkm |
| A0A2R8VJW0 | 0.33748 | −1.5671 | 0.065868 | 1.1813 | A0A2R8VJW0 | Aco2 |
| P85973 | 0.33762 | −1.5665 | 0.013318 | 1.8756 | P85973 | Pnp |
| A6J0X1 | 0.3385 | −1.5628 | 0.019763 | 1.7041 | A6J0X1 | Scarb1 |
| A0A0G2K2B3 | 0.33953 | −1.5584 | 0.010519 | 1.978 | A0A0G2K2B3 | Khsrp |
| A0A8I5ZQ28 | 0.33975 | −1.5575 | 0.00452 | 2.3448 | A0A8I5ZQ28 | Eif1 |
| A0A8J8YKQ8 | 0.34198 | −1.548 | 0.002086 | 2.6807 | A0A8J8YKQ8 | C2 |
| A6J9K8 | 0.34226 | −1.5468 | 0.003294 | 2.4823 | A6J9K8 | Sirt2 |
| A2RTT4 | 0.34237 | −1.5464 | 0.000767 | 3.1154 | A2RTT4 | Ube2n |
| Q9JHB5 | 0.34252 | −1.5457 | 0.043793 | 1.3586 | Q9JHB5 | Tsnax |
| A0A0G2K9W6 | 0.34252 | −1.5457 | 0.061065 | 1.2142 | A0A0G2K9W6 | Stam |
| A0A8I5ZXA6 | 0.34254 | −1.5456 | 0.010979 | 1.9595 | A0A8I5ZXA6 | Ndufv2 |
| Q9DBJ1 | 0.34283 | −1.5444 | 0.006431 | 2.1918 | Q9DBJ1 | Pgam1 |
| A0A8L2QD42 | 0.34373 | −1.5406 | 0.011001 | 1.9585 | A0A8L2QD42 | Sord |
| A0A8I6A583 | 0.34381 | −1.5403 | 0.004796 | 2.3191 | A0A8I6A583 | ENSRNOG0 |
| | | | | | | 0000064207 |
| A6HZ18 | 0.34458 | −1.5371 | 0.001801 | 2.7445 | A6HZ18 | Prdx5 |
| Q9DD02 | 0.34462 | −1.5369 | 0.003294 | 2.4823 | Q9DD02 | Hikeshi |
| F7FEX1 | 0.34516 | −1.5347 | 0.023918 | 1.6213 | F7FEX1 | Acadvl |
| A0A8I5ZQK2 | 0.34555 | −1.533 | 0.004622 | 2.3352 | A0A8I5ZQK2 | Tma7 |
| P18886 | 0.34727 | −1.5259 | 0.01771 | 1.7518 | P18886 | Cpt2 |
| A6JV97 | 0.34733 | −1.5256 | 0.004211 | 2.3757 | A6JV97 | Nhlrc3 |
| A0A338P692 | 0.34772 | −1.524 | 0.00314 | 2.5031 | A0A338P692 | Ahsg |
| F1LSS1 | 0.34824 | −1.5218 | 0.015434 | 1.8115 | F1LSS1 | Smc1a |
| A6JU51 | 0.34839 | −1.5212 | 0.003633 | 2.4397 | A6JU51 | Golga2 |
| A0A0R4JOS3 | 0.34914 | −1.5181 | 0.010234 | 1.99 | A0A0R4JOS3 | Rtn4ip1 |
| P46844 | 0.34916 | −1.518 | 0.003126 | 2.505 | P46844 | Blvra |
| A6HJA1 | 0.34928 | −1.5175 | 0.019977 | 1.6995 | A6HJA1 | Coa3 |
| P07633 | 0.34968 | −1.5159 | 0.035164 | 1.4539 | P07633 | Pccb |
| A0A8L2UL04 | 0.34993 | −1.5148 | 0.062182 | 1.2063 | A0A8L2UL04 | Camk1 |
| A0A0G2KB55 | 0.34998 | −1.5147 | 0.001608 | 2.7936 | A0A0G2KB55 | Ube2i |
| A0A8I6AI63 | 0.35021 | −1.5137 | 0.052472 | 1.2801 | A0A8I6AI63 | Sumo1 |
| P27139 | 0.35113 | −1.5099 | 0.0299 | 1.5243 | P27139 | Ca2 |
| A0A8L2Q0Z9 | 0.35131 | −1.5092 | 0.012965 | 1.8872 | A0A8L2Q0Z9 | Qdpr |
| A6HI41 | 0.35236 | −1.5049 | 0.008025 | 2.0956 | A6HI41 | Luc7l3 |
| A6HMR4 | 0.35242 | −1.5046 | 0.004585 | 2.3387 | A6HMR4 | Serping1 |
| A0A8I6G7K6 | 0.353 | −1.5023 | 0.060172 | 1.2206 | A0A8I6G7K6 | Arrb1 |
| A6IU84 | 0.35313 | −1.5017 | 0.012466 | 1.9043 | A6IU84 | Tardbp |
| A0A8L2QXM0 | 0.3534 | −1.5006 | 0.001108 | 2.9556 | A0A8L2QXM0 | Pea15 |
| A9UMV7 | 0.35398 | −1.4983 | 0.06688 | 1.1747 | A9UMV7 | Uqcr11 |
| Q9QYP8 | 0.35452 | −1.4961 | 0.062182 | 1.2063 | Q9QYP8 | RT1-A1b |
| A6IQ98 | 0.35543 | −1.4924 | 0.001582 | 2.8009 | A6IQ98 | rCG_36369 |
| A6KH81 | 0.35582 | −1.4908 | 0.007472 | 2.1266 | A6KH81 | Mcpt1l1 |
| D4ABK7 | 0.35674 | −1.487 | 0.006532 | 2.1849 | D4ABK7 | Hnrnph3 |
| A0A0G2K5D7 | 0.35746 | −1.4841 | 0.017755 | 1.7507 | A0A0G2K5D7 | Specc1 |
| A0A0G2K484 | 0.35749 | −1.484 | 0.098864 | 1.005 | A0A0G2K484 | Myh1 |
| P04961 | 0.35845 | −1.4802 | 0.005721 | 2.2425 | P04961 | Pcna |
| A6HKK8 | 0.35852 | −1.4799 | 0.004119 | 2.3852 | A6HKK8 | Nherf1 |
| Q8CG45 | 0.35888 | −1.4784 | 0.003186 | 2.4967 | Q8CG45 | Akr7a2 |
| A0A1S6GWH2 | 0.35896 | −1.4781 | 0.004395 | 2.3571 | A0A1S6GWH2 | Ddx39b |
| A0A8I6A888 | 0.35909 | −1.4776 | 0.009721 | 2.0123 | A0A8I6A888 | Uqcrc2 |
| D3ZSL2 | 0.36078 | −1.4708 | 0.001713 | 2.7662 | D3ZSL2 | Abracl |
| K3W4V0 | 0.3612 | −1.4691 | 0.011744 | 1.9302 | K3W4V0 | Uqcrb |
| A6IPJ5 | 0.36124 | −1.469 | 0.000634 | 3.1981 | A6IPJ5 | Idh1 |
| A6HI32 | 0.36248 | −1.464 | 0.014761 | 1.8309 | A6HI32 | rCG_34286 |
| A0A8I6AAG6 | 0.36255 | −1.4638 | 0.054681 | 1.2622 | A0A8I6AAG6 | Slc1a3 |
| A0A8I6ADP8 | 0.3629 | −1.4624 | 0.007472 | 2.1266 | A0A8I6ADP8 | Mcts1 |
| A0A0G2JTL5 | 0.36289 | −1.4624 | 0.043272 | 1.3638 | A0A0G2JTL5 | Pc |
| Q3UK30 | 0.36294 | −1.4622 | 0.006349 | 2.1973 | | |
| A6JIC0 | 0.36305 | −1.4618 | 0.017339 | 1.761 | A6JIC0 | Rftn1 |
| B1H267 | 0.36338 | −1.4605 | 0.006947 | 2.1582 | B1H267 | Snx5 |
| A0A991ENV6 | 0.36469 | −1.4552 | 0.010538 | 1.9772 | A0A991ENV6 | Sfpq |
| A0A8I6A721 | 0.3663 | −1.4489 | 0.002595 | 2.5859 | A0A8I6A721 | Mdh1 |
| A0A8L2Q919 | 0.36706 | −1.4459 | 0.020062 | 1.6976 | A0A8L2Q919 | Capg |
| A0A0G2K162 | 0.3671 | −1.4458 | 0.02719 | 1.5656 | A0A0G2K162 | Epb4112 |
| F1M978 | 0.36887 | −1.4388 | 0.002661 | 2.5749 | F1M978 | Impa1 |
| A0A8L2QBS3 | 0.36896 | −1.4385 | 0.000634 | 3.1981 | A0A8L2QBS3 | Eif5a |
| A0A8I5ZYZ4 | 0.36949 | −1.4364 | 0.008017 | 2.096 | A0A8I5ZYZ4 | Dcun1d1 |
| F7F389 | 0.36994 | −1.4346 | 0.009697 | 2.0134 | F7F389 | C9 |
| Q1RP74 | 0.37003 | −1.4343 | 0.00078 | 3.1081 | Q1RP74 | Tbcb |
| A0JPJ7 | 0.37005 | −1.4342 | 0.001261 | 2.8994 | A0JPJ7 | Ola1 |
| A0A8I6AAM9 | 0.37063 | −1.432 | 0.013937 | 1.8558 | A0A8I6AAM9 | ENSRNOG00 |
| | | | | | | 000068499 |
| G3V803 | 0.37107 | −1.4302 | 0.012732 | 1.8951 | G3V803 | Cdh2 |
| A0A8I6AMC9 | 0.37109 | −1.4302 | 0.016133 | 1.7923 | A0A8I6AMC9 | Cpamd8 |
| B5DFK6 | 0.37143 | −1.4288 | 0.050259 | 1.2988 | B5DFK6 | Ap3d1 |
| Q64194 | 0.37177 | −1.4275 | 0.023811 | 1.6232 | Q64194 | Lipa |
| Q68G49 | 0.37238 | −1.4251 | 0.017583 | 1.7549 | Q68G49 | Ces1dl1 |
| A6HXV3 | 0.37241 | −1.425 | 0.007133 | 2.1467 | A6HXV3 | Taldo1 |
| A0A8I6AB87 | 0.37253 | −1.4246 | 0.004845 | 2.3147 | A0A8I6AB87 | Gnpda1 |
| Q9CRA5 | 0.37338 | −1.4213 | 0.002274 | 2.6433 | Q9CRA5 | Golph3 |
| A6J4E7 | 0.37344 | −1.4211 | 0.005302 | 2.2755 | A6J4E7 | Dlat |
| Q570Z8 | 0.37342 | −1.4211 | 0.009159 | 2.0381 | Q570Z8 | Picalm |
| A0A8L2Q7W8 | 0.37357 | −1.4205 | 0.063806 | 1.1951 | A0A8L2Q7W8 | Gars1 |
| A6KKL4 | 0.37363 | −1.4203 | 0.095253 | 1.0211 | A6KKL4 | Gng2 |
| B5DF46 | 0.37365 | −1.4202 | 0.000476 | 3.3226 | B5DF46 | Pmm2 |
| A0A8I6GG93 | 0.37538 | −1.4136 | 0.023749 | 1.6244 | A0A8I6GG93 | Aldh1l1 |
| Q4FZY0 | 0.37631 | −1.41 | 0.03546 | 1.4503 | Q4FZY0 | Efhd2 |
| A0A8L2Q6Y2 | 0.37659 | −1.4089 | 0.018209 | 1.7397 | A0A8L2Q6Y2 | Prxl2a |
| A6JRG3 | 0.37672 | −1.4084 | 0.012732 | 1.8951 | A6JRG3 | Plaa |
| O88544 | 0.37699 | −1.4074 | 0.011411 | 1.9427 | O88544 | Cops4 |
| A0A8I5YOZ3 | 0.37763 | −1.405 | 0.004176 | 2.3793 | A0A8I5YOZ3 | Ptbp1 |
| A0A8I6AJH2 | 0.37847 | −1.4017 | 0.021571 | 1.6661 | A0A8I6AJH2 | Pdk2 |
| A0A8I6B572 | 0.37886 | −1.4003 | 0.00097 | 3.0132 | A0A8I6B572 | ENSRNOG00 |
| | | | | | | 000063840 |
| A61Y37 | 0.37902 | −1.3997 | 0.004176 | 2.3793 | A61Y37 | Tnpo2 |
| Q6AYD3 | 0.38055 | −1.3939 | 0.018228 | 1.7393 | Q6AYD3 | Pa2g4 |
| Q6MG90 | 0.38061 | −1.3936 | 0.004576 | 2.3395 | Q6MG90 | C4b |
| A0PK78 | 0.38096 | −1.3923 | 0.004725 | 2.3256 | A0PK78 | Ccdc25 |
| Q5M8A0 | 0.38102 | −1.3921 | 0.000328 | 3.4835 | Q5M8A0 | Kng2l1 |
| P27321 | 0.38209 | −1.388 | 0.049245 | 1.3076 | P27321 | Cast |
| A0A8I6AD19 | 0.38235 | −1.387 | 0.036216 | 1.4411 | A0A8I6AD19 | Flad1 |
| A0A0F7RQL3 | 0.38416 | −1.3802 | 0.014059 | 1.852 | A0A0F7RQL3 | Mif |
| P97521 | 0.38421 | −1.38 | 0.049316 | 1.307 | P97521 | Slc25a20 |
| A0A8I5ZNQ8 | 0.38488 | −1.3775 | 0.011541 | 1.9378 | A0A8I5ZNQ8 | Carhsp1 |
| F1MAA2 | 0.38509 | −1.3767 | 0.014792 | 1.83 | F1MAA2 | Cops7a |
| A0A0G2K931 | 0.38566 | −1.3746 | 0.033952 | 1.4691 | A0A0G2K931 | Psat1 |
| A6JQU0 | 0.38626 | −1.3724 | 0.035164 | 1.4539 | A6JQU0 | Ndufa10 |
| A0A8L2Q3W7 | 0.38646 | −1.3716 | 0.014533 | 1.8376 | A0A8L2Q3W7 | Pzp |
| A6KH67 | 0.38673 | −1.3706 | 0.018341 | 1.7366 | A6KH67 | Cma1 |
| A0A0G2K2P5 | 0.38714 | −1.3691 | 0.075133 | 1.1242 | A0A0G2K2P5 | Tjp1 |
| O88767 | 0.38765 | −1.3672 | 0.000328 | 3.4835 | O88767 | Park7 |
| A0A8I5ZJK8 | 0.38787 | −1.3663 | 0.0112 | 1.9508 | A0A8I5ZJK8 | Mthfd1 |
| B0K026 | 0.38847 | −1.3641 | 0.021025 | 1.6773 | B0K026 | Letmd1 |
| A0A8I6AQD7 | 0.38957 | −1.36 | 0.064856 | 1.1881 | A0A8I6AQD7 | ENSRNOG0 |
| | | | | | | 0000064041 |
| A0A8I5ZLQ4 | 0.38991 | −1.3588 | 0.002458 | 2.6095 | A0A8I5ZLQ4 | Letm1 |
| A6HCX7 | 0.38994 | −1.3587 | 0.004796 | 2.3191 | A6HCX7 | Hagh |
| A6KEC3 | 0.39038 | −1.357 | 0.025489 | 1.5936 | A6KEC3 | Pip4p1 |
| P18297 | 0.39091 | −1.3551 | 0.006843 | 2.1648 | P18297 | Spr |
| A6HXL9 | 0.39108 | −1.3545 | 0.036867 | 1.4334 | A6HXL9 | Pgghg |
| P20788 | 0.3913 | −1.3537 | 0.018851 | 1.7247 | P20788 | Uqcrfs1 |
| A6JHR8 | 0.39137 | −1.3534 | 0.00442 | 2.3546 | A6JHR8 | Gsto1 |
| D3ZUU6 | 0.39259 | −1.3489 | 0.001646 | 2.7836 | D3ZUU6 | Clec3b |
| A0A096MJY8 | 0.39315 | −1.3469 | 0.07499 | 1.125 | A0A096MJY8 | Acat2 |
| A0A3Q4EC76 | 0.39317 | −1.3468 | 0.010126 | 1.9946 | A0A3Q4EC76 | Eci1 |
| Q5BK81 | 0.39316 | −1.3468 | 0.014909 | 1.8265 | Q5BK81 | Ptgr2 |
| A6J7V5 | 0.39321 | −1.3466 | 0.019538 | 1.7091 | A6J7V5 | Alad |
| Q641W2 | 0.39323 | −1.3466 | 0.041136 | 1.3858 | Q641W2 | Myg1 |
| O08619 | 0.39366 | −1.345 | 0.011392 | 1.9434 | O08619 | F13a1 |
| A0A0G2KBC7 | 0.39445 | −1.3421 | 0.022631 | 1.6453 | A0A0G2KBC7 | Pfkm |
| Q6ZWM4 | 0.39498 | −1.3402 | 0.050264 | 1.2987 | Q6ZWM4 | Lsm8 |
| D4AB01 | 0.3958 | −1.3371 | 0.038442 | 1.4152 | D4AB01 | Hint2 |
| A6IE84 | 0.3975 | −1.331 | 0.010114 | 1.9951 | A6IE84 | Ndufa5 |
| P08010 | 0.39781 | −1.3299 | 0.00403 | 2.3947 | P08010 | Gstm2 |
| A6JRV3 | 0.39789 | −1.3296 | 0.009413 | 2.0263 | A6JRV3 | Cpn2 |
| A0A8L2UJK5 | 0.39816 | −1.3286 | 0.002034 | 2.6916 | A0A8L2UJK5 | Ccn2 |
| A6ITQ1 | 0.39835 | −1.3279 | 0.013883 | 1.8575 | A6ITQ1 | Sdhb |
| A0A0G2JV31 | 0.39839 | −1.3278 | 0.001211 | 2.917 | A0A0G2JV31 | Xpnpep1 |
| A6JAH9 | 0.39846 | −1.3275 | 0.00442 | 2.3546 | A6JAH9 | Etfb |
| A0A8I5ZTN5 | 0.3985 | −1.3273 | 0.00442 | 2.3546 | A0A8I5ZTN5 | Adsl |
| B1WBN3 | 0.39878 | −1.3263 | 0.018889 | 1.7238 | B1WBN3 | Bckdha |
| Q5EBC0 | 0.39902 | −1.3255 | 0.000366 | 3.4371 | Q5EBC0 | Itih4 |
| A0A8I5ZME8 | 0.39911 | −1.3251 | 0.02675 | 1.5727 | A0A8I5ZME8 | Puf60 |
| B0BNN3 | 0.40004 | −1.3218 | 0.063526 | 1.197 | B0BNN3 | Ca1 |
| Q5U300 | 0.40036 | −1.3206 | 0.000507 | 3.2947 | Q5U300 | Uba1 |
| A0A8I5ZN09 | 0.40126 | −1.3174 | 0.054103 | 1.2668 | A0A8I5ZN09 | Sugt1 |
| Q9Z210 | 0.40153 | −1.3164 | 0.004348 | 2.3618 | Q9Z210 | Letm1 |
| A6HY44 | 0.40164 | −1.316 | 0.003639 | 2.439 | A6HY44 | Ctsd |
| B5DER4 | 0.4018 | −1.3155 | 0.000917 | 3.0374 | B5DER4 | Mrpl1 |
| F1LN07 | 0.40193 | −1.315 | 0.037262 | 1.4287 | F1LN07 | Scgn |
| A612B1 | 0.40206 | −1.3145 | 0.02776 | 1.5566 | A612B1 | Nmnat3 |
| A6JLZ9 | 0.4021 | −1.3144 | 0.061972 | 1.2078 | A6JLZ9 | Sephs1 |
| Q61990 | 0.40211 | −1.3143 | 0.009148 | 2.0387 | Q61990 | Pcbp2 |
| Q9JJ54 | 0.40254 | −1.3128 | 0.002657 | 2.5755 | Q9JJ54 | Hnrnpd |
| A0A0G2K626 | 0.40337 | −1.3098 | 0.012962 | 1.8873 | A0A0G2K626 | Sec24c |
| A6JT83 | 0.40357 | −1.3091 | 0.002598 | 2.5854 | A6JT83 | Phpt1 |
| A6IV43 | 0.40367 | −1.3088 | 0.00372 | 2.4294 | A6IV43 | Pgk1 |
| P08503 | 0.40428 | −1.3066 | 0.011847 | 1.9264 | P08503 | Acadm |
| A0A8I6AMJ9 | 0.4044 | −1.3062 | 0.036612 | 1.4364 | A0A8I6AMJ9 | Cpsf6 |
| Q9Z0J5 | 0.40443 | −1.306 | 0.055517 | 1.2556 | Q9Z0J5 | Txnrd2 |
| G3V9U2 | 0.40449 | −1.3058 | 0.043651 | 1.36 | G3V9U2 | Acaa2 |
| A616M6 | 0.40499 | −1.3041 | 0.002008 | 2.6973 | A616M6 | Ppme1 |
| Q9JLZ1 | 0.40564 | −1.3017 | 0.003238 | 2.4898 | Q9JLZ1 | Glrx3 |
| A0A8L2Q447 | 0.40644 | −1.2989 | 0.00335 | 2.4749 | A0A8L2Q447 | Galm |
| A6IRU5 | 0.40763 | −1.2947 | 0.020557 | 1.687 | A6IRU5 | Pak2 |
| A0A991ENW0 | 0.40788 | −1.2938 | 0.038376 | 1.4159 | A0A991ENW0 | H1f10 |
| A0A0G2JY66 | 0.40792 | −1.2936 | 0.012843 | 1.8913 | A0A0G2JY66 | Ces1d |
| A6KMH2 | 0.4081 | −1.293 | 0.018339 | 1.7366 | A6KMH2 | Ppif |
| A6K9W5 | 0.40822 | −1.2926 | 0.06998 | 1.155 | A6K9W5 | Pgls_predicted |
| Q923W4 | 0.4083 | −1.2923 | 0.004585 | 2.3387 | Q923W4 | Hdgfl3 |
| D4A9N5 | 0.40874 | −1.2907 | 0.054194 | 1.266 | D4A9N5 | Trim25 |
| F7FG85 | 0.40907 | −1.2896 | 0.045445 | 1.3425 | F7FG85 | Man2b1 |
| A0A8I6AE43 | 0.40934 | −1.2886 | 0.08447 | 1.0733 | A0A8I6AE43 | Erbin |
| A0A8I6AJE6 | 0.40999 | −1.2863 | 0.00907 | 2.0424 | A0A8I6AJE6 | Decr2 |
| A6J219 | 0.41033 | −1.2852 | 0.061432 | 1.2116 | A6J219 | Mapre2 |
| F1LQS6 | 0.4107 | −1.2839 | 0.019538 | 1.7091 | F1LQS6 | Xdh |
| A6K4P2 | 0.41097 | −1.2829 | 0.089718 | 1.0471 | A6K4P2 | Ppl |
| F1LQ48 | 0.41156 | −1.2808 | 0.006431 | 2.1918 | F1LQ48 | Hnrnpl |
| B2GVB9 | 0.41188 | −1.2797 | 0.029203 | 1.5346 | B2GVB9 | Fermt3 |
| A61047 | 0.4127 | −1.2768 | 0.002661 | 2.5749 | A61047 | Ddb1 |
| E9PY39 | 0.41335 | −1.2746 | 0.001103 | 2.9576 | E9PY39 | Gm20431 |
| D3ZD11 | 0.41344 | −1.2743 | 0.031948 | 1.4956 | D3ZD11 | Spcs2 |
| A6JF15 | 0.41388 | −1.2727 | 0.083592 | 1.0778 | A6JF15 | Atp6v1h |
| F7FLI1 | 0.41409 | −1.272 | 0.059594 | 1.2248 | F7FLI1 | Lbp |
| A0A8I6A790 | 0.41454 | −1.2704 | 0.001744 | 2.7584 | A0A8I6A790 | Clic1 |
| A0A0G2K3Z9 | 0.41473 | −1.2698 | 0.00047 | 3.3281 | A0A0G2K3Z9 | Prdx1l1 |
| A0A9K3Y7E2 | 0.41471 | −1.2698 | 0.013495 | 1.8698 | A0A9K3Y7E2 | Uqcr10 |
| F1LRV6 | 0.41506 | −1.2686 | 0.021728 | 1.663 | F1LRV6 | Gmpr |
| D3ZPL2 | 0.41544 | −1.2673 | 0.08986 | 1.0464 | D3ZPL2 | ENSRNOG |
| | | | | | | 00000063422 |
| O70492 | 0.41591 | −1.2656 | 0.001582 | 2.8009 | O70492 | Snx3 |
| Q5VLR6 | 0.41654 | −1.2635 | 0.014787 | 1.8301 | | |
| D3ZVQ0 | 0.41656 | −1.2634 | 0.008527 | 2.0692 | D3ZVQ0 | Usp5 |
| P10760 | 0.41658 | −1.2633 | 0.000987 | 3.0056 | P10760 | Ahcy |
| A0A8I5ZQ10 | 0.41695 | −1.262 | 0.09639 | 1.016 | A0A8I5ZQ10 | Naa50 |
| A0A8I6AES4 | 0.417 | −1.2619 | 0.005677 | 2.2459 | A0A8I6AES4 | Ctss |
| A0A8I6ATZ3 | 0.41729 | −1.2609 | 0.026071 | 1.5838 | A0A8I6ATZ3 | Cisd3 |
| Q3UGB5 | 0.41776 | −1.2593 | 0.010842 | 1.9649 | Q3UGB5 | Dazap1 |
| A6JIA5 | 0.41794 | −1.2586 | 0.009536 | 2.0206 | A6JIA5 | Eif3a |
| A0A8L2QFW5 | 0.41819 | −1.2578 | 0.04991 | 1.3018 | A0A8L2QFW5 | Mrps26 |
| A619E6 | 0.41868 | −1.2561 | 0.01091 | 1.9622 | A619E6 | Aldoa |
| A0A8I6AC39 | 0.41874 | −1.2559 | 0.063686 | 1.196 | A0A8I6AC39 | Acsl3 |
| Q3ULN8 | 0.41928 | −1.254 | 0.043272 | 1.3638 | Q3ULN8 | Ppp2r5a |
| A0A0G2KAW7 | 0.41937 | −1.2537 | 0.010234 | 1.99 | A0A0G2KAW7 | Eif4h |
| A0A9K3Y6Z3 | 0.41996 | −1.2517 | 0.011744 | 1.9302 | A0A9K3Y6Z3 | Hebp1 |
| A6KDI8 | 0.42124 | −1.2473 | 0.074743 | 1.1264 | A6KDI8 | rCG_21034 |
| P23514 | 0.42126 | −1.2472 | 0.011276 | 1.9479 | P23514 | Copb1 |
| A0A8I6AD89 | 0.42244 | −1.2432 | 0.03642 | 1.4387 | A0A8I6AD89 | ENSRNOG0 |
| | | | | | | 0000064245 |
| D4A962 | 0.42283 | −1.2419 | 0.005818 | 2.2352 | D4A962 | Hnrnpul1 |
| G3V837 | 0.42332 | −1.2402 | 0.008527 | 2.0692 | G3V837 | Cd1d1 |
| A6K8E8 | 0.4241 | −1.2375 | 0.003732 | 2.4281 | A6K8E8 | Lsm7_predicted |
| B0K010 | 0.42458 | −1.2359 | 0.001386 | 2.8581 | B0K010 | Txndc17 |
| P04636 | 0.42562 | −1.2324 | 0.001499 | 2.8243 | P04636 | Mdh2 |
| A0A8I5YC99 | 0.4263 | −1.2301 | 0.026658 | 1.5742 | A0A8I5YC99 | Eps15 |
| Q61206 | 0.42646 | −1.2295 | 0.003699 | 2.4319 | Q61206 | Pafah1b2 |
| Q5FVH2 | 0.42683 | −1.2283 | 0.057178 | 1.2428 | Q5FVH2 | Pld3 |
| A0A8I6A1R3 | 0.42745 | −1.2262 | 0.027799 | 1.556 | A0A8I6A1R3 | Retsat |
| Q9DC70 | 0.42786 | −1.2248 | 0.016591 | 1.7801 | Q9DC70 | Ndufs7 |
| P19357 | 0.42852 | −1.2226 | 0.016232 | 1.7896 | P19357 | Slc2a4 |
| F1LTN6 | 0.42954 | −1.2191 | 0.009125 | 2.0398 | F1LTN6 | AABR07 |
| | | | | | | 060872.1 |
| A0A8I6AC45 | 0.43034 | −1.2165 | 0.091043 | 1.0408 | A0A8I6AC45 | Impdh1 |
| A0A8I6AJ25 | 0.43059 | −1.2156 | 0.081134 | 1.0908 | A0A8I6AJ25 | Bpifb1 |
| F1LXA0 | 0.43118 | −1.2136 | 0.005818 | 2.2352 | F1LXA0 | Ndufa12 |
| A6J7C8 | 0.43194 | −1.2111 | 0.005095 | 2.2929 | A6J7C8 | F13a1 |
| A0A077S116 | 0.43202 | −1.2108 | 0.011934 | 1.9232 | A0A077S116 | Lyz2 |
| A6JS43 | 0.43349 | −1.2059 | 0.00047 | 3.3281 | A6JS43 | Hrg |
| P04355 | 0.43351 | −1.2059 | 0.039591 | 1.4024 | P04355 | Mt2 |
| A0A8I5Y6N4 | 0.43404 | −1.2041 | 0.086807 | 1.0614 | A0A8I5Y6N4 | Parvb |
| A0A8I6AB78 | 0.43406 | −1.204 | 0.094792 | 1.0232 | A0A8I6AB78 | Lypla2 |
| G3V9N0 | 0.43441 | −1.2029 | 0.02452 | 1.6105 | G3V9N0 | Pabpc4 |
| A0A096MJT0 | 0.43515 | −1.2004 | 0.004622 | 2.3352 | A0A096MJT0 | Cacybp |
| A6KDI1 | 0.43523 | −1.2001 | 0.027607 | 1.559 | A6KDI1 | rCG_21092 |
| QOOP19 | 0.43524 | −1.2001 | 0.061065 | 1.2142 | Q00P19 | Hnrnpul2 |
| A2NW55 | 0.43597 | −1.1977 | 0.093368 | 1.0298 | | |
| A0A8I6AG01 | 0.43639 | −1.1963 | 0.011321 | 1.9461 | A0A8I6AG01 | Nedd8 |
| F1LM47 | 0.43644 | −1.1962 | 0.008665 | 2.0623 | F1LM47 | Sucla2 |
| P15650 | 0.43672 | −1.1952 | 0.018208 | 1.7397 | P15650 | Acadl |
| A0A8I6AI37 | 0.4383 | −1.19 | 0.011276 | 1.9479 | A0A8I6AI37 | Snrpd3 |
| A6HK92 | 0.43901 | −1.1877 | 0.001801 | 2.7445 | A6HK92 | Apoh |
| A0A0U1RS25 | 0.43969 | −1.1855 | 0.089229 | 1.0495 | A0A0U1RS25 | Upf1 |
| A0A8I6AB50 | 0.44057 | −1.1826 | 0.012594 | 1.8998 | A0A8I6AB50 | Ada |
| Q60587 | 0.4414 | −1.1798 | 0.012093 | 1.9175 | Q60587 | Hadhb |
| Q9D1K2 | 0.44259 | −1.176 | 0.037262 | 1.4287 | Q9D1K2 | Atp6v1f |
| Q01986 | 0.44401 | −1.1713 | 0.033362 | 1.4767 | Q01986 | Map2k1 |
| A0A8I5ZWI8 | 0.44457 | −1.1695 | 0.02776 | 1.5566 | A0A8I5ZWI8 | Dnajc19 |
| D4AE56 | 0.44498 | −1.1682 | 0.079663 | 1.0987 | D4AE56 | Ptges2 |
| A6IX75 | 0.44573 | −1.1658 | 0.002034 | 2.6916 | A6IX75 | Prrc1 |
| A0A140TAH1 | 0.44581 | −1.1655 | 0.033093 | 1.4803 | A0A140TAH1 | Hgs |
| Q4QQV4 | 0.44629 | −1.164 | 0.02064 | 1.6853 | Q4QQV4 | Hars1 |
| P61971 | 0.44656 | −1.1631 | 0.001103 | 2.9576 | P61971 | Nutf2 |
| A0A8J8XSI7 | 0.44715 | −1.1612 | 0.006349 | 2.1973 | A0A8J8XSI7 | Oas1a |
| F6X4N5 | 0.44713 | −1.1612 | 0.017024 | 1.7689 | | |
| A0A8L2Q6N7 | 0.44717 | −1.1611 | 0.014056 | 1.8521 | A0A8L2Q6N7 | Caprin1 |
| F1LS86 | 0.44747 | −1.1601 | 0.016337 | 1.7868 | F1LS86 | lars1 |
| Q9Z269 | 0.44764 | −1.1596 | 0.005806 | 2.2361 | Q9Z269 | Vapb |
| D3ZC54 | 0.44775 | −1.1592 | 0.051083 | 1.2917 | D3ZC54 | AABR07 |
| | | | | | | 065823.2 |
| F7FF93 | 0.44786 | −1.1589 | 0.009721 | 2.0123 | F7FF93 | Arsa |
| Q6IG11 | 0.44862 | −1.1564 | 0.085007 | 1.0705 | Q6IG11 | Krt81 |
| A6IKS7 | 0.44881 | −1.1558 | 0.038427 | 1.4154 | A6IKS7 | Ogdh |
| A0A8L2QTB7 | 0.45032 | −1.151 | 0.010631 | 1.9734 | A0A8L2QTB7 | Cox7a2 |
| A0A8I6GKW3 | 0.45038 | −1.1508 | 0.005677 | 2.2459 | A0A8I6GKW3 | Nans |
| Q811A2 | 0.4511 | −1.1485 | 0.005607 | 2.2513 | Q811A2 | Bst2 |
| Q7TMC3 | 0.4516 | −1.1469 | 0.012414 | 1.9061 | Q7TMC3 | Saa4 |
| Q4V8H9 | 0.45238 | −1.1444 | 0.079232 | 1.1011 | Q4V8H9 | Ifit2 |
| A0A0G2JVL6 | 0.45289 | −1.1428 | 0.014059 | 1.852 | A0A0G2JVL6 | Ndufa8 |
| A0A8I6A4W2 | 0.45293 | −1.1426 | 0.005677 | 2.2459 | A0A8I6A4W2 | Ktn1 |
| A6K246 | 0.45311 | −1.1421 | 0.009615 | 2.0171 | A6K246 | Steap4 |
| Q8BPF4 | 0.45312 | −1.142 | 0.037038 | 1.4314 | | |
| A0A0G2JT06 | 0.45365 | −1.1403 | 0.000675 | 3.1707 | A0A0G2JT06 | Gps1 |
| A0A8I6AS83 | 0.45393 | −1.1395 | 0.011541 | 1.9378 | A0A8I6AS83 | Tfrc |
| A0A8I5ZVD5 | 0.45431 | −1.1382 | 0.003177 | 2.4979 | A0A8I5ZVD5 | Dnajc8 |
| B2GUV5 | 0.45442 | −1.1379 | 0.036216 | 1.4411 | B2GUV5 | Atp6v1g1 |
| P17563 | 0.45441 | −1.1379 | 0.040961 | 1.3876 | P17563 | Selenbp1 |
| A6KRR0 | 0.45521 | −1.1354 | 0.010035 | 1.9985 | A6KRR0 | Gdi1 |
| D3YTQ3 | 0.45594 | −1.1331 | 0.054194 | 1.266 | D3YTQ3 | Hnrnpdl |
| P53987 | 0.45663 | −1.1309 | 0.01771 | 1.7518 | P53987 | Slc16a1 |
| A0A8I5Y0X7 | 0.45775 | −1.1274 | 0.016337 | 1.7868 | A0A8I5Y0X7 | Lrrfip1 |
| A6IT06 | 0.4584 | −1.1253 | 0.006185 | 2.2087 | A6IT06 | Sh3bgrl3 |
| A0A8I5ZUV1 | 0.45859 | −1.1247 | 0.036692 | 1.4354 | A0A8I5ZUV1 | Cox4i1 |
| A6J7X0 | 0.4587 | −1.1244 | 0.005415 | 2.2664 | A6J7X0 | Ambp |
| F1LW91 | 0.45947 | −1.122 | 0.004585 | 2.3387 | F1LW91 | Numa1 |
| B2RYP4 | 0.4608 | −1.1178 | 0.00509 | 2.2933 | B2RYP4 | Snx2 |
| Q80ZA3 | 0.46092 | −1.1174 | 0.012351 | 1.9083 | Q80ZA3 | Serpinf1 |
| F1M6X7 | 0.46127 | −1.1163 | 0.063662 | 1.1961 | F1M6X7 | Arhgap17 |
| A0A8I5ZTU5 | 0.46143 | −1.1158 | 0.002274 | 2.6433 | A0A8I5ZTU5 | Ranbp1 |
| Q8VH51 | 0.46149 | −1.1156 | 0.018341 | 1.7366 | Q8VH51 | Rbm39 |
| A0A8I5ZXC8 | 0.46311 | −1.1106 | 0.004995 | 2.3014 | A0A8I5ZXC8 | Mrps16 |
| A0A8I6AJF4 | 0.46318 | −1.1104 | 0.018208 | 1.7397 | A0A8I6AJF4 | Ube2v2 |
| Q5BK33 | 0.46386 | −1.1082 | 0.017458 | 1.758 | Q5BK33 | Mpp1 |
| A0A8I6A6C3 | 0.46524 | −1.1039 | 0.072625 | 1.1389 | A0A8I6A6C3 | ENSRNOG0 |
| | | | | | | 0000067603 |
| A0A8I5Y7D7 | 0.4653 | −1.1038 | 0.047922 | 1.3195 | A0A8I5Y7D7 | LOC134 |
| | | | | | | 483981 |
| A0A8J8XPQ6 | 0.46548 | −1.1032 | 0.02929 | 1.5333 | A0A8J8XPQ6 | Dcps |
| P53812-2 | 0.46567 | −1.1026 | 0.014056 | 1.8521 | | |
| A0A0G2JYJ7 | 0.46573 | −1.1024 | 0.008321 | 2.0798 | A0A0G2JYJ7 | Rbms2 |
| B1WC32 | 0.46667 | −1.0995 | 0.030822 | 1.5111 | B1WC32 | Uba2 |
| Q68FT7 | 0.46704 | −1.0984 | 0.003109 | 2.5075 | Q68FT7 | Farsb |
| P05370 | 0.46729 | −1.0976 | 0.00049 | 3.3099 | P05370 | G6pdx |
| A0A8I6AHS3 | 0.46736 | −1.0974 | 0.004067 | 2.3907 | A0A8I6AHS3 | Txnl1 |
| Q3VOZ8 | 0.46792 | −1.0957 | 0.02835 | 1.5474 | Q3V0Z8 | Ddx5 |
| A6J9L4 | 0.46793 | −1.0956 | 0.046858 | 1.3292 | A6J9L4 | Ech1 |
| P56812 | 0.46822 | −1.0947 | 0.008696 | 2.0607 | P56812 | Pdcd5 |
| G3V8D2 | 0.46937 | −1.0912 | 0.024331 | 1.6138 | G3V8D2 | Prx |
| F7FFD0 | 0.46944 | −1.091 | 0.022902 | 1.6401 | F7FFD0 | Timp3 |
| A0A8I5YBK9 | 0.46952 | −1.0907 | 0.017616 | 1.7541 | A0A8I5YBK9 | Xirp1 |
| P35235-1 | 0.46955 | −1.0906 | 0.016827 | 1.774 | | |
| Q921M3 | 0.46973 | −1.0901 | 0.035641 | 1.448 | Q921M3 | Sf3b3 |
| A0A8I5ZDN9 | 0.47094 | −1.0864 | 0.019957 | 1.6999 | A0A8I5ZDN9 | C5 |
| Q5M9G9 | 0.47151 | −1.0846 | 0.012752 | 1.8944 | Q5M9G9 | Tbrg4 |
| A0A8I6AL00 | 0.47156 | −1.0845 | 0.053039 | 1.2754 | A0A8I6AL00 | Ndufs8 |
| Q920F5 | 0.4721 | −1.0828 | 0.06955 | 1.1577 | Q920F5 | Mlycd |
| D4A7D7 | 0.47264 | −1.0812 | 0.077713 | 1.1095 | D4A7D7 | H6pd |
| Q6AXY0 | 0.4731 | −1.0798 | 0.006168 | 2.2099 | Q6AXY0 | Gsta6 |
| D3ZVM5 | 0.47387 | −1.0774 | 0.050717 | 1.2948 | D3ZVM5 | Hspa12b |
| A0A8I5ZU95 | 0.47463 | −1.0751 | 0.050264 | 1.2987 | A0A8I5ZU95 | Tmem126a |
| A6HBY8 | 0.475 | −1.074 | 0.050264 | 1.2987 | A6HBY8 | Pygl |
| D3YXF5 | 0.47586 | −1.0714 | 0.000917 | 3.0374 | D3YXF5 | C7 |
| P38656 | 0.47593 | −1.0712 | 0.010489 | 1.9793 | P38656 | Ssb |
| Q6IMZ5 | 0.4766 | −1.0691 | 0.004119 | 2.3852 | Q6IMZ5 | Tmod1 |
| D3ZE08 | 0.47664 | −1.069 | 0.011744 | 1.9302 | D3ZE08 | ENSRNOG00 |
| | | | | | | 000065564 |
| F1M9A7 | 0.47696 | −1.0681 | 0.020872 | 1.6804 | F1M9A7 | Acox3 |
| A6JC64 | 0.47709 | −1.0677 | 0.033059 | 1.4807 | A6JC64 | Plin1 |
| A0A8I6A2B3 | 0.4783 | −1.064 | 0.003816 | 2.4184 | A0A8I6A2B3 | Guk1 |
| A6KIK8 | 0.47874 | −1.0627 | 0.018228 | 1.7393 | A6KIK8 | Akap12 |
| P15327 | 0.47919 | −1.0613 | 0.065747 | 1.1821 | P15327 | Bpgm |
| P30349 | 0.47979 | −1.0595 | 0.025607 | 1.5916 | P30349 | Lta4h |
| P62311 | 0.48011 | −1.0586 | 0.007072 | 2.1504 | P62311 | Lsm3 |
| A0A8I6A5G9 | 0.48039 | −1.0577 | 0.010489 | 1.9793 | A0A8I6A5G9 | Gm2a |
| P62962 | 0.48046 | −1.0575 | 0.00047 | 3.3281 | P62962 | Pfn1 |
| A6JE02 | 0.48083 | −1.0564 | 0.015771 | 1.8021 | A6JE02 | DIst |
| P45591 | 0.48164 | −1.054 | 0.001743 | 2.7586 | P45591 | Cfl2 |
| A0A8I5ZZ13 | 0.48186 | −1.0533 | 0.063686 | 1.196 | A0A8I5ZZ13 | Ppa2 |
| Q5UT85 | 0.48274 | −1.0507 | 0.081277 | 1.09 | Q5UT85 | RT1-Ba |
| Q3TDF8 | 0.48285 | −1.0504 | 0.012262 | 1.9114 | Q3TDF8 | Etf |
| A6JB99 | 0.48299 | −1.0499 | 0.036794 | 1.4342 | A6JB99 | Tmem143 |
| Q9CSU0 | 0.48381 | −1.0475 | 0.083329 | 1.0792 | Q9CSU0 | Rprd1b |
| A0A8I6AM99 | 0.48424 | −1.0462 | 0.085323 | 1.0689 | A0A8I6AM99 | Ppp1r7 |
| A0A8I5ZY32 | 0.48432 | −1.046 | 0.012648 | 1.898 | A0A8I5ZY32 | Ehbp1l1 |
| Q3ZAV2 | 0.48429 | −1.046 | 0.017003 | 1.7695 | Q3ZAV2 | Ybx1 |
| A0A0H2UHF8 | 0.48434 | −1.0459 | 0.001801 | 2.7445 | A0A0H2UHF8 | Orm1 |
| Q3UPA3 | 0.48434 | −1.0459 | 0.004786 | 2.3201 | Q3UPA3 | Gdi2 |
| D4A4U3 | 0.48447 | −1.0455 | 0.001801 | 2.7445 | D4A4U3 | Mdp1 |
| Q5RKI0 | 0.48451 | −1.0454 | 0.009936 | 2.0028 | Q5RKI0 | Wdr1 |
| A0A8J8XBZ3 | 0.48577 | −1.0416 | 0.008527 | 2.0692 | A0A8J8XBZ3 | Parp3 |
| F1M9V7 | 0.48645 | −1.0396 | 0.057974 | 1.2368 | F1M9V7 | Npepps |
| A0A8I6A9H4 | 0.48654 | −1.0394 | 0.091516 | 1.0385 | A0A8I6A9H4 | Art3 |
| Q9JHL4 | 0.48712 | −1.0377 | 0.009721 | 2.0123 | Q9JHL4 | Dbnl |
| Q2MHH0 | 0.4873 | −1.0371 | 0.040134 | 1.3965 | Q2MHH0 | Trarg1 |
| F7FKI8 | 0.48828 | −1.0342 | 0.008429 | 2.0742 | F7FKI8 | Hspb7 |
| A0A0G2JX93 | 0.49041 | −1.0279 | 0.016632 | 1.779 | A0A0G2JX93 | Stat1 |
| D3ZH41 | 0.49125 | −1.0255 | 0.091972 | 1.0363 | D3ZH41 | Ckap4 |
| Q9D868 | 0.49196 | −1.0234 | 0.03744 | 1.4267 | Q9D868 | Ppih |
| D3Z5F7 | 0.49247 | −1.0219 | 0.005677 | 2.2459 | D3Z5F7 | Gm20521 |
| Q63707 | 0.49282 | −1.0209 | 0.018785 | 1.7262 | Q63707 | Dhodh |
| Q3TIQ3 | 0.49338 | −1.0192 | 0.002464 | 2.6084 | Q3TIQ3 | Pitpna |
| A0A8I5ZPN3 | 0.4939 | −1.0177 | 0.011001 | 1.9585 | A0A8I5ZPN3 | Map2k2 |
| Q9JLT5 | 0.49408 | −1.0172 | 0.017315 | 1.7616 | Q9JLT5 | Wfs1 |
| A6HZN1 | 0.49413 | −1.017 | 0.001801 | 2.7445 | A6HZN1 | Stip1 |
| Q3UUU2 | 0.49414 | −1.017 | 0.02163 | 1.6649 | Q3UUU2 | Fubp1 |
| Q9WV02 | 0.49528 | −1.0137 | 0.01108 | 1.9555 | Q9WV02 | Rbmx |
| Q9QZA6 | 0.49547 | −1.0131 | 0.004619 | 2.3354 | Q9QZA6 | Cd151 |
| A0A8I6A906 | 0.49556 | −1.0129 | 0.020216 | 1.6943 | A0A8I6A906 | Eif3g |
| A0JPK5 | 0.4956 | −1.0127 | 0.062182 | 1.2063 | A0JPK5 | Abhd5 |
| A6HAE4 | 0.49599 | −1.0116 | 0.023262 | 1.6334 | A6HAE4 | Hadha |
| Q921A4 | 0.49763 | −1.0068 | 0.054396 | 1.2644 | Q921A4 | Cygb |
| Q3U8W9 | 0.49781 | −1.0063 | 0.044586 | 1.3508 | Q3U8W9 | Hnrnpr |
| A6HNR6 | 0.49929 | −1.002 | 0.043082 | 1.3657 | A6HNR6 | Cat |
| Q510D7 | 0.49976 | −1.0007 | 0.012262 | 1.9114 | Q510D7 | Pepd |
| A0A8I6GLL0 | 0.49977 | −1.0007 | 0.038376 | 1.4159 | A0A8I6GLL0 | C4bpb |
| Q9Z1H9 | 2.0079 | 1.0057 | 0.023396 | 1.6309 | Q9Z1H9 | Cavin3 |
| Q5RKI5 | 2.0123 | 1.0088 | 0.005806 | 2.2361 | Q5RKI5 | Flii |
| A0A8I6GJJ3 | 2.0163 | 1.0117 | 0.022751 | 1.643 | A0A8I6GJJ3 | Stat3 |
| A0A8I6A0L3 | 2.0204 | 1.0146 | 0.005677 | 2.2459 | A0A8I6A0L3 | Tmem43 |
| Q6T487 | 2.0262 | 1.0188 | 0.008905 | 2.0504 | Q6T487 | Actn1 |
| D3ZC19 | 2.0289 | 1.0207 | 0.018228 | 1.7393 | D3ZCI9 | Myl10 |
| Q1A602 | 2.0405 | 1.0289 | 0.021571 | 1.6661 | Q1A602 | Actn4 |
| A6ID16 | 2.0435 | 1.031 | 0.03209 | 1.4936 | A6ID16 | Fam20b |
| D3ZDQ9 | 2.0481 | 1.0343 | 0.036094 | 1.4426 | D3ZDQ9 | Sgca |
| A0A8I5Y510 | 2.0517 | 1.0368 | 0.067942 | 1.1679 | A0A8I5Y510 | Capn2 |
| A0A8I5ZXA1 | 2.0527 | 1.0375 | 0.005818 | 2.2352 | A0A8I5ZXA1 | Myl12a |
| A0A8L2Q617 | 2.0626 | 1.0444 | 0.009721 | 2.0123 | A0A8L2Q617 | Dad1 |
| F1LS40 | 2.0691 | 1.049 | 0.097888 | 1.0093 | F1LS40 | Col1a2 |
| A6K9Q7 | 2.0695 | 1.0493 | 0.026071 | 1.5838 | A6K9Q7 | Tpm4 |
| P62737 | 2.0725 | 1.0513 | 0.002464 | 2.6084 | P62737 | Acta2 |
| A0A8I6GFI0 | 2.0907 | 1.064 | 0.017024 | 1.7689 | A0A8I6GFI0 | Dcn |
| Q52L67 | 2.091 | 1.0642 | 0.086764 | 1.0617 | Q52L67 | Tecr |
| A0A8I6AAP2 | 2.1194 | 1.0836 | 0.010739 | 1.969 | A0A8I6AAP2 | ENSRNOG00 |
| | | | | | | 000063112 |
| Q9CQ19 | 2.1193 | 1.0836 | 0.094792 | 1.0232 | Q9CQ19 | Myl9 |
| A0A8I6GJY7 | 2.1272 | 1.0889 | 0.036101 | 1.4425 | A0A8I6GJY7 | Snx9 |
| P97449 | 2.1358 | 1.0948 | 0.048217 | 1.3168 | P97449 | Anpep |
| Q3U926 | 2.1362 | 1.095 | 0.010489 | 1.9793 | Q3U926 | Ptpmt1 |
| B1WC61 | 2.1361 | 1.095 | 0.033362 | 1.4767 | B1WC61 | Acad9 |
| Q9EQP5 | 2.1475 | 1.1027 | 0.008696 | 2.0607 | Q9EQP5 | Prelp |
| A0A8L2Q0U6 | 2.1482 | 1.1031 | 0.061697 | 1.2097 | A0A8L2Q0U6 | Pycr2 |
| G3V9Y9 | 2.1509 | 1.1049 | 0.081325 | 1.0898 | G3V9Y9 | Ap3s1 |
| F1M6W2 | 2.153 | 1.1064 | 0.062082 | 1.207 | F1M6W2 | Ermp1 |
| A6KAG2 | 2.1581 | 1.1098 | 0.086559 | 1.0627 | A6KAG2 | Gas1 |
| A0A8I6A609 | 2.1718 | 1.1189 | 0.021587 | 1.6658 | A0A8I6A609 | Gpc6 |
| A6K3J1 | 2.1772 | 1.1225 | 0.011662 | 1.9332 | A6K3J1 | Tspan2 |
| B2RYD7 | 2.179 | 1.1236 | 0.050674 | 1.2952 | B2RYD7 | Stt3b |
| A6KNQ6 | 2.1887 | 1.13 | 0.023749 | 1.6244 | A6KNQ6 | Ssc5d |
| F6T7Z2 | 2.194 | 1.1335 | 0.07161 | 1.145 | F6T7Z2 | Tmem119 |
| A0A0G2KAT5 | 2.2049 | 1.1407 | 0.05107 | 1.2918 | A0A0G2KAT5 | Ptk2 |
| A6JTC7 | 2.211 | 1.1447 | 0.043602 | 1.3605 | A6JTC7 | Qsox2 |
| Q3TWV0 | 2.2218 | 1.1517 | 0.040436 | 1.3932 | Q3TWV0 | Vim |
| A0A096MK61 | 2.2282 | 1.1559 | 0.029386 | 1.5319 | A0A096MK61 | Crtap |
| A6IWJ9 | 2.2342 | 1.1598 | 0.024883 | 1.6041 | A6IWJ9 | Proz_predicted |
| A0A8I6AL43 | 2.2383 | 1.1624 | 0.029386 | 1.5319 | A0A8I6AL43 | Ate1 |
| A6KMB1 | 2.2407 | 1.164 | 0.004619 | 2.3354 | A6KMB1 | Lama5 |
| Q8R4A1 | 2.2531 | 1.1719 | 0.06955 | 1.1577 | Q8R4A1 | Ero1a |
| A6K3V1 | 2.2723 | 1.1841 | 0.008346 | 2.0785 | A6K3V1 | Mcu |
| Q6LC76 | 2.2754 | 1.1861 | 0.030797 | 1.5115 | Q6LC76 | fn-1 |
| P82349 | 2.2825 | 1.1906 | 0.01375 | 1.8617 | P82349 | Sgcb |
| A0A8I6AC07 | 2.2844 | 1.1918 | 0.012811 | 1.8924 | A0A8I6AC07 | Sfxn1 |
| Q5M823 | 2.291 | 1.196 | 0.012648 | 1.898 | Q5M823 | Nudcd2 |
| Q69ZX3 | 2.3024 | 1.2031 | 0.020835 | 1.6812 | Q69ZX3 | Myh11 |
| G3V6E7 | 2.3061 | 1.2055 | 0.01406 | 1.852 | G3V6E7 | Fmod |
| Q3KR94 | 2.3186 | 1.2132 | 0.01399 | 1.8542 | Q3KR94 | Vtn |
| Q66HA8 | 2.3212 | 1.2149 | 0.036222 | 1.441 | Q66HA8 | Hsph1 |
| B2RZ77 | 2.3261 | 1.2179 | 0.014792 | 1.83 | B2RZ77 | Dpt |
| A6KSD5 | 2.3283 | 1.2193 | 0.001801 | 2.7445 | A6KSD5 | rCG_42490 |
| A0A0G2KAE1 | 2.3504 | 1.2329 | 0.036222 | 1.441 | A0A0G2KAE1 | Lims2 |
| A6J2Z8 | 2.3633 | 1.2408 | 0.039591 | 1.4024 | A6J2Z8 | Cxxc5 |
| D3ZAI6 | 2.3793 | 1.2506 | 0.022902 | 1.6401 | D3ZAI6 | Nt5dc3 |
| Q8CFG7 | 2.3838 | 1.2532 | 0.003201 | 2.4948 | Q8CFG7 | Cacna2d1 |
| Q642B0 | 2.3994 | 1.2627 | 0.012648 | 1.898 | Q642B0 | Gpc4 |
| A6JTK8 | 2.4002 | 1.2631 | 0.004652 | 2.3324 | A6JTK8 | Sardh |
| A6K450 | 2.4101 | 1.2691 | 0.04254 | 1.3712 | A6K450 | Srgn |
| F7EWJ6 | 2.4135 | 1.2712 | 0.018799 | 1.7259 | F7EWJ6 | Nt5e |
| A0A8I6AEQ6 | 2.4221 | 1.2763 | 0.024948 | 1.603 | A0A8I6AEQ6 | ENSRNOG0 |
| | | | | | | 0000068254 |
| A0A213BQG3 | 2.4275 | 1.2795 | 0.031587 | 1.5005 | A0A213BQG3 | Spcs1 |
| A0A0G2K6S9 | 2.4388 | 1.2862 | 0.007133 | 2.1467 | A0A0G2K6S9 | Myh11 |
| A6IJ58 | 2.4526 | 1.2943 | 0.005644 | 2.2484 | A6IJ58 | Reck |
| P70490 | 2.4609 | 1.2992 | 0.020503 | 1.6882 | P70490 | Mfge8 |
| A6KD08 | 2.4669 | 1.3027 | 0.009512 | 2.0217 | A6KD08 | Itga5 |
| E9PZ16 | 2.4767 | 1.3084 | 0.021229 | 1.6731 | E9PZ16 | Hspg2 |
| A0A8J8XAG4 | 2.4911 | 1.3168 | 0.005677 | 2.2459 | A0A8J8XAG4 | Itpr1 |
| A0A0G2K695 | 2.5054 | 1.3251 | 0.015353 | 1.8138 | A0A0G2K695 | Myof |
| A0A0G2KAJ7 | 2.5439 | 1.347 | 0.012594 | 1.8998 | A0A0G2KAJ7 | Col12a1 |
| P97927 | 2.5481 | 1.3494 | 0.011276 | 1.9479 | P97927 | Lama4 |
| A0A8I5ZMG0 | 2.5481 | 1.3494 | 0.025489 | 1.5936 | A0A8I5ZMG0 | Tmod2 |
| Q8VHS9 | 2.5521 | 1.3517 | 0.009372 | 2.0282 | Q8VHS9 | Cacna2d1 |
| A0A0A6YXI2 | 2.5566 | 1.3542 | 0.087337 | 1.0588 | A0A0A6YXI2 | Fabp9 |
| A6IMC9 | 2.5676 | 1.3604 | 0.023811 | 1.6232 | A6IMC9 | rCG_29673 |
| O54698 | 2.5804 | 1.3676 | 0.040727 | 1.3901 | 054698 | Slc29a1 |
| E9PSY0 | 2.5909 | 1.3735 | 0.045557 | 1.3414 | E9PSY0 | Axl |
| B2GV54 | 2.6039 | 1.3807 | 0.008905 | 2.0504 | B2GV54 | Nceh1 |
| D4ABF0 | 2.6139 | 1.3862 | 0.004585 | 2.3387 | D4ABF0 | P3h4 |
| F7EXA4 | 2.6354 | 1.398 | 0.071357 | 1.1466 | F7EXA4 | Mx1 |
| Q8BRW2 | 2.6641 | 1.4137 | 0.009721 | 2.0123 | Q8BRW2 | Adipoq |
| A0A0G2K052 | 2.6654 | 1.4144 | 0.024331 | 1.6138 | A0A0G2K052 | Ano1 |
| A6JED2 | 2.666 | 1.4147 | 0.039989 | 1.3981 | A6JED2 | Sel1l |
| D3ZA76 | 2.6682 | 1.4159 | 0.037329 | 1.428 | D3ZA76 | Htra3 |
| Q6P6T0 | 2.674 | 1.419 | 0.001261 | 2.8994 | Q6P6T0 | Sfxn3 |
| Q571B0 | 2.6774 | 1.4209 | 0.000366 | 3.4371 | Q571B0 | Tm9sf3 |
| A0A8I6AMG2 | 2.6865 | 1.4257 | 0.012752 | 1.8944 | A0A8I6AMG2 | Bgn |
| A6JL62 | 2.7255 | 1.4465 | 0.017107 | 1.7668 | A6JL62 | App |
| A0A0G2K7B6 | 2.7345 | 1.4513 | 0.039533 | 1.403 | A0A0G2K7B6 | Dysf |
| 14DUB5 | 2.7363 | 1.4522 | 0.037358 | 1.4276 | 14DUB5 | Itga3 |
| O08564 | 2.7546 | 1.4618 | 0.023298 | 1.6327 | O08564 | Plpp1 |
| Q5PQQ8 | 2.7634 | 1.4664 | 0.020432 | 1.6897 | Q5PQQ8 | Itgbl1 |
| D4ACX8 | 2.7686 | 1.4692 | 0.006676 | 2.1755 | D4ACX8 | Dchs1 |
| A0A8I5ZN98 | 2.7895 | 1.48 | 0.019957 | 1.6999 | A0A8I5ZN98 | Agps |
| O35786 | 2.7954 | 1.483 | 0.011276 | 1.9479 | O35786 | Cmklr1 |
| E9QA15 | 2.7964 | 1.4836 | 0.031115 | 1.507 | E9QA15 | Cald1 |
| D4A447 | 2.8085 | 1.4898 | 0.006947 | 2.1582 | D4A447 | Cd109 |
| F1LNS2 | 2.8201 | 1.4957 | 0.008483 | 2.0714 | F1LNS2 | Dennd10 |
| A0A1S7IVG9 | 2.8629 | 1.5175 | 0.040454 | 1.393 | A0A1S7IVG9 | Vkorc1 |
| A0A8I6AJ52 | 2.8723 | 1.5222 | 0.044753 | 1.3492 | A0A8I6AJ52 | Ap1m1 |
| P47819 | 2.8787 | 1.5254 | 0.027799 | 1.556 | P47819 | Gfap |
| A6IAI6 | 2.8938 | 1.533 | 0.039912 | 1.3989 | A6IAI6 | Loxl3 |
| A0A0G2K9X1 | 3.0274 | 1.5981 | 0.022902 | 1.6401 | A0A0G2K9X1 | Spp2 |
| A6JN89 | 3.0575 | 1.6124 | 0.03997 | 1.3983 | A6JN89 | Endod1 |
| A6HAT8 | 3.0702 | 1.6183 | 0.010538 | 1.9772 | A6HAT8 | Slc66a3 |
| A61205 | 3.2006 | 1.6783 | 0.003242 | 2.4892 | A61205 | Tmed3 |
| Q9QZK5 | 3.28 | 1.7137 | 0.005456 | 2.2632 | Q9QZK5 | Htra1 |
| A0A8I6ASG6 | 3.2844 | 1.7157 | 0.037262 | 1.4287 | A0A8I6ASG6 | Sdc2 |
| A6J3W5 | 3.3996 | 1.7653 | 0.005563 | 2.2547 | A6J3W5 | NIrx1 |
| Q8CG09 | 3.439 | 1.782 | 0.013883 | 1.8575 | Q8CG09 | Abcc1 |
| A0A8I6ATN0 | 3.4616 | 1.7914 | 0.021409 | 1.6694 | A0A8I6ATN0 | Stum |
| B5DF94 | 3.4896 | 1.8031 | 0.015231 | 1.8173 | B5DF94 | Srpx2 |
| Q60847 | 3.5107 | 1.8117 | 0.024871 | 1.6043 | Q60847 | Col12a1 |
| A6JYG2 | 3.5397 | 1.8236 | 0.008346 | 2.0785 | A6JYG2 | Aplp2 |
| Q8BNY6 | 3.5594 | 1.8316 | 0.010114 | 1.9951 | Q8BNY6 | Ncs1 |
| P63005 | 3.5752 | 1.838 | 0.06955 | 1.1577 | P63005 | Pafah1b1 |
| P24090 | 3.6673 | 1.8747 | 0.009721 | 2.0123 | P24090 | Ahsg |
| A6HBX3 | 3.6772 | 1.8786 | 0.043272 | 1.3638 | A6HBX3 | Dmac2l |
| A0A8I5ZYJ7 | 3.6909 | 1.884 | 0.004176 | 2.3793 | A0A8I5ZYJ7 | Podn |
| A0A8L2Q098 | 3.7705 | 1.9148 | 0.055517 | 1.2556 | A0A8L2Q098 | Kdelr2 |
| A0A0G2JSI0 | 3.8207 | 1.9338 | 0.005302 | 2.2755 | A0A0G2JSI0 | Fmo3 |
| Q9JHY2 | 3.8336 | 1.9387 | 0.005095 | 2.2929 | Q9JHY2 | Sfxn3 |
| Q4FZU6 | 3.8794 | 1.9558 | 0.087798 | 1.0565 | Q4FZU6 | Anxa8 |
| A0A0G2JTI8 | 4.2173 | 2.0763 | 0.00078 | 3.1081 | A0A0G2JTI8 | Enpep |
| Q3UAA9 | 4.4204 | 2.1442 | 0.006349 | 2.1973 | Q3UAA9 | Actb |
| A0A7U3JWB2 | 4.5986 | 2.2012 | 0.030984 | 1.5089 | A0A7U3JWB2 | Fgf8b |
| F7EZZ6 | 4.6533 | 2.2183 | 0.021229 | 1.6731 | F7EZZ6 | Pla2g4a |
| A6KA56 | 4.8449 | 2.2765 | 0.038442 | 1.4152 | A6KA56 | Comp |
| A6JAM8 | 5.0784 | 2.3444 | 0.023396 | 1.6309 | A6JAM8 | Clec11a |
| F7FMY6 | 5.4719 | 2.452 | 0.018438 | 1.7343 | F7FMY6 | Proc |
| Q8K3U6 | 5.4755 | 2.453 | 0.084411 | 1.0736 | Q8K3U6 | F7 |
| A6KA48 | 5.7159 | 2.515 | 0.043272 | 1.3638 | A6KA48 | Crlf1 |
| P11530 | 5.7956 | 2.5349 | 0.026071 | 1.5838 | P11530 | Dmd |
| A6ITI9 | 5.8086 | 2.5382 | 0.021229 | 1.6731 | A6ITI9 | Pla2g2a |
| A6IMJ2 | 6.1223 | 2.6141 | 0.018228 | 1.7393 | A6IMJ2 | Mgp |
| P08721 | 6.7923 | 2.7639 | 0.019133 | 1.7182 | P08721 | Spp1 |
| A0A8I5ZXW2 | 7.7833 | 2.9604 | 0.000352 | 3.454 | A0A8I5ZXW2 | Hmga1 |
| A6JC41 | 7.8509 | 2.9729 | 0.073221 | 1.1354 | A6JC41 | Acan |
| M0RB67 | 7.9561 | 2.9921 | 0.006349 | 2.1973 | M0RB67 | Ppidl1 |
| P14152 | 8.7027 | 3.1215 | 0.005975 | 2.2237 | P14152 | Mdh1 |
| F7EU69 | 9.0557 | 3.1788 | 0.003242 | 2.4892 | F7EU69 | Lancl2 |
| A6KGY1 | 11.186 | 3.4836 | 0.000366 | 3.4371 | A6KGY1 | Myh7 |
| A0A8I6AEF5 | 20.026 | 4.3238 | 0.000675 | 3.1707 | A0A8I6AEF5 | Ankh |

Bioinformatic enrichment analysis using the DAVID platform identified several disease phenotypes potentially amenable to EDTA-based therapeutic intervention, including glycogen storage disorders, primary mitochondrial pathologies, age-related macular degeneration, hereditary hemolytic anemias, peripheral neuropathies, neurodegenerative conditions, syndromic disease variants, and hemolytic uremic syndrome. Table 2 is a list of conditions. Tables 3-9 list genes involved in their progression that were downregulated by EDTA treatment. All these aging related disorders are characterized by mitochondrial dysfunction, metabolic stress, and chronic inflammation—hallmarks that overlap mechanistically with vascular calcification and the senescence-associated secretory phenotype (SASP).

TABLE 2
List of Conditions Amenable to EDTA NP Therapy

| | Gene | | P- | Benjamini- |
|---|---|---|---|---|
| Condition | Count | % | Value | Hochberg CV |
| Disease Variant | 202 | 34.4 | 9.50E−02 | 8.50E−01 |
| Neurodegeneration | 29 | 4.9 | 8.20E−02 | 8.50E−01 |
| Primary Mitochondrial | 22 | 3.7 | 9.50E−04 | 3.30E−02 |
| Disease | | | | |
| Neuropathy | 13 | 2.2 | 3.90E−02 | 5.50E−01 |
| Glycogen Storage Disease | 8 | 1.4 | 5.50E−05 | 3.80E−03 |
| Hereditary Hemolytic | 7 | 1.2 | 1.70E−02 | 3.00E−01 |
| Anemia | | | | |
| Age-Related Macular | 5 | 0.9 | 6.30E−03 | 1.50E−01 |
| Degeneration | | | | |
| Hemolytic Uremic | 3 | 0.5 | 9.70E−02 | 8.50E−01 |
| Syndrome | | | | |

For the general disease variants in Table 2, 202 genes downregulated by EDTA NPs were identified.

For neurodegeneration, 29 genes were identified, shown below in Table 3.

TABLE 3
Genes amenable to EDTA NP therapy related to neurodegeneration

| Gene | |
|---|---|
| Symbol | Gene Name |
| Htra2 | HtrA serine peptidase 2(HTRA2) |
| Park7 | Parkinsonism associated deglycase(PARK7) |
| Tardbp | TAR DNA binding protein(TARDBP) |
| Vapb | VAMP associated protein B and C(VAPB) |
| Aco2 | aconitase 2(ACO2) |
| Crat | carnitine O-acetyltransferase(CRAT) |
| Ctsd | cathepsin D(CTSD) |
| Caprin1 | cell cycle associated protein 1(CAPRIN1) |
| Coq7 | coenzyme Q7, hydroxylase(COQ7) |
| Coq8a | coenzyme Q8A(COQ8A) |
| Coa7 | cytochrome c oxidase assembly factor 7(COA7) |
| Cox6a1 | cytochrome c oxidase subunit 6A1(COX6A1) |
| Eif4g1 | eukaryotic translation initiation factor 4 gamma 1(EIF4G1) |
| Gars1 | glycyl-tRNA synthetase 1 (GARS1) |
| Hars1 | histidyl-tRNA synthetase 1(HARS1) |
| Letm1 | leucine zipper and EF-hand containing transmembrane protein |
| | 1 (LETM1) |
| Matr3 | matrin 3(MATR3) |
| Prx | periaxin(PRX) |
| Farsb | phenylalanyl-tRNA synthetase subunit beta(FARSB) |
| Pld3 | phospholipase D family member 3(PLD3) |
| Pfn1 | profilin 1(PFN1) |
| Pcna | proliferating cell nuclear antigen(PCNA) |
| Ppp5c | protein phosphatase 5 catalytic subunit(PPP5C) |
| Pdxk | pyridoxal kinase(PDXK) |
| Sirt2 | sirtuin 2(SIRT2) |
| Sod1 | superoxide dismutase 1(SOD1) |
| Yars1 | tyrosyl-tRNA synthetase 1(YARS1) |
| Uchl1 | ubiquitin C-terminal hydrolase L1(UCHL1) |
| Uba1 | ubiquitin like modifier activating enzyme 1 (UBA1) |

For primary mitochondrial disease, 22 genes were identified, shown below in Table 4.

TABLE 4
Genes amenable to EDTA NP therapy related
to primary mitochondrial disease

| Gene | |
|---|---|
| Symbol | Gene Name |
| Ndufs7 | NADH:ubiquinone oxidoreductase core subunit S7(NDUFS7) |
| Ndufs8 | NADH:ubiquinone oxidoreductase core subunit S8(NDUFS8) |
| Ndufv2 | NADH:ubiquinone oxidoreductase core subunit V2(NDUFV2) |
| Ndufa10 | NADH:ubiquinone oxidoreductase subunit A10(NDUFA10) |
| Ndufa12 | NADH:ubiquinone oxidoreductase subunit A12(NDUFA12) |
| Ndufa8 | NADH:ubiquinone oxidoreductase subunit A8(NDUFA8) |
| Coq5 | coenzyme Q5, methyltransferase(COQ5) |
| Coq6 | coenzyme Q6, monooxygenase(COQ6) |
| Coq7 | coenzyme Q7, hydroxylase(COQ7) |
| Coq8a | coenzyme Q8A(COQ8A) |
| Coq9 | coenzyme Q9(COQ9) |
| Coa3 | cytochrome c oxidase assembly factor 3(COA3) |
| Cox4i1 | cytochrome c oxidase subunit 4I1(COX411) |
| Cox6b1 | cytochrome c oxidase subunit 6B1(COX6B1) |
| Cyc1 | cytochrome c1(CYC1) |
| Flad1 | flavin adenine dinucleotide synthetase 1(FLAD1) |
| Mrps16 | mitochondrial ribosomal protein S16(MRPS16) |
| Sdhb | succinate dehydrogenase complex iron sulfur subunit |
| | B(SDHB) |
| Sucla2 | succinate-CoA ligase ADP-forming subunit beta(SUCLA2) |
| Uqcrb | ubiquinol-cytochrome c reductase binding protein(UQCRB) |
| Uqcrc2 | ubiquinol-cytochrome c reductase core protein 2(UQCRC2) |
| Uqcrfs1 | ubiquinol-cytochrome c reductase, Rieske iron-sulfur |
| | polypeptide 1(UQCRFS1) |

For neuropathy, 13 genes were identified, shown below in Table 5.

TABLE 5
Genes amenable to EDTA NP therapy related to neuropathy

| Gene | |
|---|---|
| Symbol | Gene Name |
| Gbe1 | 1,4-alpha-glucan branching enzyme |
| | 1(GBE1) |
| Acox1 | acyl-CoA oxidase 1(ACOX1) |
| Coq7 | coenzyme Q7, hydroxylase(COQ7) |
| Coa7 | cytochrome c oxidase assembly factor |
| | 7(COA7) |
| Cox6a1 | cytochrome c oxidase subunit |
| | 6A1(COX6A1) |
| Gars1 | glycyl-tRNA synthetase 1(GARS1) |
| Hars1 | histidyl-tRNA synthetase 1(HARS1) |
| Prx | periaxin(PRX) |
| Pdxk | pyridoxal kinase(PDXK) |
| Rpia | ribose 5-phosphate isomerase A(RPIA) |
| Sord | sorbitol dehydrogenase(SORD) |
| Ttr | transthyretin(TTR) |
| Yars1 | tyrosyl-tRNA synthetase 1(YARS1) |

For glycogen storage disease, 8 genes were identified, shown below in Table 6.

TABLE 6
Genes amenable to EDTA NP therapy
related to glycogen storage disease

| Gene | |
|---|---|
| Symbol | Gene Name |
| Gbe1 | 1,4-alpha-glucan branching enzyme |
| | 1(GBE1) |
| Aldoa | aldolase, fructose-bisphosphate |
| | A(ALDOA) |
| Eno3 | enolase 3(ENO3) |
| Pygl | glycogen phosphorylase L(PYGL) |
| Ldha | lactate dehydrogenase A(LDHA) |
| Pfkm | phosphofructokinase, muscle(PFKM) |
| Pgm1 | phosphoglucomutase 1(PGM1) |
| Pgam2 | phosphoglycerate mutase 2(PGAM2) |

For hereditary hemolytic anemia, 7 genes were identified, shown below in Table 7.

TABLE 7
Genes amenable to EDTA NP therapy related
to hereditary hemolytic anemia

| Gene | |
|---|---|
| Symbol | Gene Name |
| Ada | adenosine deaminase(ADA) |
| Ak1 | adenylate kinase 1(AK1) |
| Aldoa | aldolase, fructose-bisphosphate |
| | A(ALDOA) |
| Bpgm | bisphosphoglycerate mutase(BPGM) |
| Gpi | glucose-6-phosphate |
| | isomerase(GPI) |
| Gss | glutathione synthetase(GSS) |
| Pgk1 | phosphoglycerate kinase 1(PGK1) |

For age-related macular degeneration, 5 genes were identified, shown below in Table 8.

TABLE 8
Genes amenable to EDTA NP therapy related
to age-related macular degeneration

| Gene | |
|---|---|
| Symbol | Gene Name |
| C2 | complement C2(C2) |
| C3 | complement C3(C3) |
| C9 | complement C9(C9) |
| Cfb | complement factor |
| | B(CFB) |
| Cfi | complement factor |
| | I(CFI) |

For hemolytic uremic syndrome, 3 genes were identified, shown below in Table 9.

TABLE 9
Genes amenable to EDTA NP therapy related
to hemolytic uremic syndrome

| Gene | |
|---|---|
| Symbol | Gene Name |
| C3 | complement C3(C3) |
| Cfb | complement factor |
| | B(CFB) |
| Cfi | complement factor |
| | I(CFI) |

Further analysis with DAVID (online bioinformatics tool from NIH) revealed that EDTA-NP treatment majorly exerts its therapeutic effect via regulating the Post Translational Modifications on proteins involved in lipid metabolism and TCA cycle (acetylation, phosphorylation and methylation; Table 10). Such modifications are implicated in a spectrum of pathological conditions relevant to vascular aging and inflammation. This independent bioinformatics analysis strengthens the claim that targeted EDTA-NP delivery not only attenuates calcific remodeling but can also mitigate molecular features associated with systemic aging associated degenerative disorders and inflammatory diseases.

TABLE 10
Enzymes involved in Post Translational
Modifications downregulated by EDTA

| | Gene | | | Benjamini- |
|---|---|---|---|---|
| Term | Count | % | P-Value | Hochberg CV |
| Acetylation | 323 | 55 | 9.3E−75 | 2.8E−73 |
| Phosphoprotein | 367 | 62.5 | 0.000000089 | 0.0000013 |
| Methylation | 58 | 9.9 | 0.002 | 0.02 |
| Hydroxylation | 17 | 2.9 | 0.0036 | 0.027 |
| S-nitrosylation | 7 | 1.2 | 0.038 | 0.23 |
| Glutathionylation | 3 | 0.5 | 0.069 | 0.34 |
| Glycation | 3 | 0.5 | 0.08 | 0.34 |
| Thioester bond | 3 | 0.5 | 0.091 | 0.34 |

Further, biological processes were altered by EDTA treatment, shown in Tables 11 and 12 below.

TABLE 11
Biological Processes Altered by EDTA treatment (Cluster 2)

| | | | | Benjamini- |
|---|---|---|---|---|
| Annotation | Enrichment | | | Hochberg |
| Cluster 2 | Score: 9.43 | Count | P_Value | CV |
| Goterm_bp_{—} | Glycolytic | 18 | 2.9E−14 | 7.4E−11 |
| direct | Process | | | |
| Up_kw | Glycolysis | 15 | 2.5E−12 | 1.8E−10 |
| biological_{—} | | | | |
| process | | | | |
| Kegg_pathway | Glycolysis/ | 21 | 6.5E−12 | 6.7E−10 |
| | Gluconeogenesis | | | |
| Goterm_bp_{—} | Canonical | 11 | 1.2E−10 | 0.0000001 |
| direct | Glycolysis | | | |
| Kegg_pathway | Biosynthesis | 20 | 5.1E−10 | 0.000000032 |
| | of Amino Acids | | | |
| Goterm_bp_{—} | Gluconeogenesis | 12 | 0.000000085 | 0.000023 |
| direct | | | | |
| Kegg_pathway | HIF-1 Signaling | 15 | 0.00041 | 0.0049 |
| | Pathway | | | |

TABLE 12
Biological Processes Altered by EDTA treatment (Cluster 3)

| | | | | Benjamini- |
|---|---|---|---|---|
| Annotation | Enrichment | | | Hochberg |
| Cluster 3 | Score: 9.26 | Count | P_Value | CV |
| Up_Kw | Fatty Acid | 28 | 2.7E−12 | 1.8E−10 |
| Biological_{—} | Metabolism | | | |
| Process | | | | |
| Goterm_Bp_Direct | Fatty | 15 | 7.9E−11 | 0.0000001 |
| | Acid Beta- | | | |
| | Oxidation | | | |
| Kegg_Pathway | Fatty Acid | 16 | 2.5E−10 | 0.000000019 |
| | Degradation | | | |
| Goterm_Bp_Direct | Fatty Acid | 19 | 5.9E−09 | 0.0000025 |
| | Metabolic | | | |
| | Process | | | |
| Kegg_Pathway | Fatty Acid | 15 | 0.00000015 | 0.000004 |
| | Metabolism | | | |

What is claimed is:

1. A method for reducing senescent cell accumulation, the method comprising:
delivering a nanoparticle comprising a chelating agent to a tissue.

2. The method of claim 1, wherein the nanoparticle comprises a liposome.

3. The method of claim 1, wherein the nanoparticle comprises a protein.

4. The method of claim 1, wherein the nanoparticle comprises a polymer.

5. The method of claim 2, wherein the liposome comprises a multilamellar vesicle.

6. The method of claim 1, wherein the chelating agent comprises 4 wt. % to 40 wt. % of the nanoparticle.

7. The method of claim 1, further comprising delivering the chelating agent to a tissue according to a dosing regimen.

8. The method of claim 1, wherein the delivering a nanoparticle comprises administering the nanoparticle after a patient evaluation.

9. The method of claim 1, wherein the chelating agent comprises EDTA, EGTA, 1PTA, NTA, IDS, EDDS, polyaspartic acid, MGDA, L-glutamic acid, N,N-diacetic acid, or GLDA, citric acid, and salts thereof.

10. The method of claim 1, wherein the nanoparticles further comprise an antibody.

11. The method of claim 10, wherein the antibody comprises one or more of SEQ ID NO: 9, SEQ ID NO: 11, SEQ ID NO: 13, SEQ ID NO: 27, SEQ ID NO: 29, and SEQ ID NO: 31.

12. A method for treating a patient with a condition, the method comprising:
administering a nanoparticle comprising a chelating agent to the patient.

13. The method of claim 12, further comprising administering the nanoparticle to the patient in a dosing regimen.

14. The method of claim 12, wherein the nanoparticle is administered to the patient in an amount of 2 mg/kg of body weight to 50 mg/kg of body weight.

15. The method of claim 12, wherein the nanoparticle is administered to the patient intravenously.

16. The method of claim 12, wherein the chelating agent comprises 4 wt. % to 40 wt. % of the nanoparticle.

17. The method of claim 12, wherein the condition is one of macular degeneration, vascular calcification, atherosclerosis or chronic kidney disease.

18. The method of claim 12, wherein the chelating agent comprises EDTA, EGTA, 1PTA, NTA, IDS, EDDS, polyaspartic acid, MGDA, L-glutamic acid, N,N-diacetic acid, or GLDA, citric acid, and salts thereof.

19. The method of claim 12, wherein the nanoparticle comprises a liposome and wherein the liposome has a negative surface charge.

20. The method of claim 12, wherein the nanoparticle further comprises an antibody.

21. The method of claim 20, wherein the antibody comprises one or more of SEQ ID NO: 9, SEQ ID NO: 11, SEQ ID NO: 13, SEQ ID NO: 27, SEQ ID NO: 29, and SEQ ID NO: 31.

22. The method of claim 12, wherein the nanoparticle is disposed within an excipient.

23. The method of claim 12, wherein the nanoparticle is delivered to the patient with systemic delivery or local delivery.


SEQUENCE LISTING (41 sequences)

SEQ ID NO: 1 - protein, 17 aa, Rattus sp.
GALGPGGKPPKPGAGLL

SEQ ID NO: 2 - protein, 47 aa, Rattus sp.
LGYPIKAPKLPGGYGLPYTTGKLPYGYPGGVAGAAGKAGYPTTGTGV

SEQ ID NO: 3 - protein, 18 aa, unidentified
  REGION 1..18: note = Description of Unknown: Elastin sequence
PGGYGLPYTTGKLPYGYP

SEQ ID NO: 4 - DNA, 1368 bp, synthetic construct
  misc_feature 1..1368: note = Description of Artificial Sequence: Synthetic polynucleotide
  source 1..1368: mol_type = other DNA
atggcttgggtgtggaccttgctattcctgatggcagctgcccaaagtgcccaagcacagatccagttggagcagtctggacctgagctgaagaagcctggagagacagtcaagatctcctgcaaggcttctgggtataccttcagaaagtatggaatgagctgggtgaagcaggctccaggaaaacatttaaagtggatgggctggataaacacctacactggaaagccaacatatgctgatgacttcaagggacggtttgccttctctttgggaacctctgccagcactgcctatttgcagatcaacaacctcagaaatgaggacacggctacatatttctgtgcaagagaagactactggggccaaggcaccactctcacagtctcctcagccaaaacgacacccccatctgtctatccactggcccctggatctgctgcccaaactaactccatggtgaccctgggatgcctggtcaagggctatttccctgagccagtgacagtgacctggaactctggatccctgtccagcggtgtgcacaccttcccagctgtcctgcagtctgacctctacactctgagcagctcagtgactgtcccctccagcacctggcccagcgagaccgtcacctgcaacgttgcccacccggccagcagcaccaaggtggacaagaaaattgtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccactctcctggtaaatga

SEQ ID NO: 5 - protein, 455 aa, synthetic construct
  REGION 1..455: note = Description of Artificial Sequence: Synthetic polypeptide
MAWVWTLLFLMAAAQSAQAQIQLEQSGPELKKPGETVKISCKASGYTFRKYGMSWVKQAPGKHLKWMGWINTYTGKPTYADDFKGRFAFSLGTSASTAYLQINSLRNEDTATYFCAREDYWGQGTTLTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 6 - DNA, 336 bp, synthetic construct
  misc_feature 1..336: note = Description of Artificial Sequence: Synthetic polynucleotide
  source 1..336: mol_type = other DNA
cagatccagttggagcagtctggacctgagctgaagaagcctggagagacagtcaagatctcctgcaaggcttctgggtataccttcagaaagtatggaatgagctgggtgaagcaggctccaggaaaacatttaaagtggatgggctggataaacacctacactggaaagccaacatatgctgatgacttcaagggacggtttgccttctctttgggaacctctgccagcactgcctatttgcagatcaacaacctcagaaatgaggacacggctacatatttctgtgcaagagaagactactggggccaaggcaccactctcacagtctcctca

SEQ ID NO: 7 - protein, 112 aa, synthetic construct
  REGION 1..112: note = Description of Artificial Sequence: Synthetic polypeptide
QIQLEQSGPELKKPGETVKISCKASGYTFRKYGMSWVKQAPGKHLKWMGWINTYTGKPTYADDFKGRFAFSLGTSASTAYLQINNLRNEDTATYFCAREDYWGQGTTLTVSS

SEQ ID NO: 8 - DNA, 15 bp, synthetic construct
  misc_feature 1..15: note = Description of Artificial Sequence: Synthetic oligonucleotide
  source 1..15: mol_type = other DNA
aagtatggaatgagc

SEQ ID NO: 9 - protein, 5 aa, synthetic construct
  REGION 1..5: note = Description of Artificial Sequence: Synthetic peptide
KYGMS

SEQ ID NO: 10 - DNA, 51 bp, synthetic construct
  misc_feature 1..51: note = Description of Artificial Sequence: Synthetic oligonucleotide
  source 1..51: mol_type = other DNA
tggataaacacctacactggaaagccaacatatgctgatgacttcaaggga

SEQ ID NO: 11 - protein, 17 aa, synthetic construct
  REGION 1..17: note = Description of Artificial Sequence: Synthetic peptide
WINTYTGKPTYADDFKG

SEQ ID NO: 12-13 - skipped (2 entries)

SEQ ID NO: 14 - DNA, 90 bp, synthetic construct
  misc_feature 1..90: note = Description of Artificial Sequence: Synthetic oligonucleotide
  source 1..90: mol_type = other DNA
cagatccagttggagcagtctggacctgagctgaagaagcctggagagacagtcaagatctcctgcaaggcttctgggtataccttcaga

SEQ ID NO: 15 - protein, 30 aa, synthetic construct
  REGION 1..30: note = Description of Artificial Sequence: Synthetic polypeptide
QIQLEQSGPELKKPGETVKISCKASGYTFR

SEQ ID NO: 16 - DNA, 42 bp, synthetic construct
  misc_feature 1..42: note = Description of Artificial Sequence: Synthetic oligonucleotide
  source 1..42: mol_type = other DNA
tgggtgaagcaggctccaggaaaacatttaaagtggatgggc

SEQ ID NO: 17 - protein, 14 aa, synthetic construct
  REGION 1..14: note = Description of Artificial Sequence: Synthetic peptide
WVKQAPGKHLKWMG

SEQ ID NO: 18 - DNA, 96 bp, synthetic construct
  misc_feature 1..96: note = Description of Artificial Sequence: Synthetic oligonucleotide
  source 1..96: mol_type = other DNA
cggtttgccttctctttgggaacctctgccagcactgcctatttgcagatcaacaacctcagaaatgaggacacggctacatatttctgtgcaaga

SEQ ID NO: 19 - protein, 32 aa, synthetic construct
  REGION 1..32: note = Description of Artificial Sequence: Synthetic polypeptide
RFAFSLGTSASTAYLQINNLRNEDTATYFCAR

SEQ ID NO: 20 - DNA, 33 bp, synthetic construct
  misc_feature 1..33: note = Description of Artificial Sequence: Synthetic oligonucleotide
  source 1..33: mol_type = other DNA
tggggccaaggcaccactctcacagtctcctca

SEQ ID NO: 21 - protein, 11 aa, synthetic construct
  REGION 1..11: note = Description of Artificial Sequence: Synthetic peptide
WGQGTTLTVSS

SEQ ID NO: 22 - DNA, 717 bp, synthetic construct
  misc_feature 1..717: note = Description of Artificial Sequence: Synthetic polynucleotide
  source 1..717: mol_type = other DNA
atgagtcctgcccagttcctgtttctgttagtgctctggattcgggaaaccaacggtgatgttgtgatgacccagactccactcactttgtcggttaccattggacaaccagcctccatctcttgcaagtcaggtcagagcctcttaaatagtgatggaaagacatatttgaattggttgttacagcggccaggccagtctccaaagcgcctaatctatctggtgtctaaactggactctggagtccctgacaggttcactggcagtggatcagggacagatttcacactaaaaatcagcagagtggaggctgaggatttgggagtttattattgctggcaaggtacacattttccgtggacgttcggtggaggcaccaagctggaaatcaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgttag

SEQ ID NO: 23 - protein, 237 aa, synthetic construct
  REGION 1..237: note = Description of Artificial Sequence: Synthetic polypeptide
MSPAQFLFLLVLWIRETNGDVVMTQTPLTLSVTIGQPASISCKSGQSLLNSDGKTYLNWLLQRPGQSPKRLIYLVSKLDSGVPDRFTGSGSGTDFTLKISRVEAEDLGVYYCWQGTHFPWTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNE

SEQ ID NO: 24 - DNA, 336 bp, synthetic construct
  misc_feature 1..336: note = Description of Artificial Sequence: Synthetic polynucleotide
  source 1..336: mol_type = other DNA
gatgttgtgatgacccagactccactcactttgtcggttaccattggacaaccagcctccatctcttgcaagtcaggtcagagcctcttaaatagtgatggaaagacatatttgaattggttgttacagcggccaggccagtctccaaagcgcctaatctatctggtgtctaaactggactctggagtccctgacaggttcactggcagtggatcagggacagatttcacactaaaaatcagcagagtggaggctgaggatttgggagtttattattgctggcaaggtacacattttccgtggacgttcggtggaggcaccaagctggaaatcaaa

SEQ ID NO: 25 - protein, 112 aa, synthetic construct
  REGION 1..112: note = Description of Artificial Sequence: Synthetic polypeptide
DVVMTQTPLTLSVTIGQPASISCKSGQSLLNSDGKTYLNWLLQRPGQSPKRLIYLVSKLDSGVPDRFTGSGSGTDFTLKISRVEAEDLGVYYCWQGTHFPWTFGGGTKLEIK

SEQ ID NO: 26 - DNA, 48 bp, synthetic construct
  misc_feature 1..48: note = Description of Artificial Sequence: Synthetic oligonucleotide
  source 1..48: mol_type = other DNA
aagtcaggtcagagcctcttaaatagtgatggaaagacatatttgaat

SEQ ID NO: 27 - protein, 16 aa, synthetic construct
  REGION 1..16: note = Description of Artificial Sequence: Synthetic peptide
KSGQSLLNSDGKTYLN

SEQ ID NO: 28 - DNA, 21 bp, synthetic construct
  misc_feature 1..21: note = Description of Artificial Sequence: Synthetic oligonucleotide
  source 1..21: mol_type = other DNA
ctggtgtctaaactggactct

SEQ ID NO: 29 - protein, 7 aa, synthetic construct
  REGION 1..7: note = Description of Artificial Sequence: Synthetic peptide
LVSKLDS

SEQ ID NO: 30 - DNA, 27 bp, synthetic construct
  misc_feature 1..27: note = Description of Artificial Sequence: Synthetic oligonucleotide
  source 1..27: mol_type = other DNA
tggcaaggtacacattttccgtggacg

SEQ ID NO: 31 - protein, 9 aa, synthetic construct
  REGION 1..9: note = Description of Artificial Sequence: Synthetic peptide
WQGTHFPWT

SEQ ID NO: 32 - DNA, 69 bp, synthetic construct
  misc_feature 1..69: note = Description of Artificial Sequence: Synthetic oligonucleotide
  source 1..69: mol_type = other DNA
gatgttgtgatgacccagactccactcactttgtcggttaccattggacaaccagcctccatctcttgc

SEQ ID NO: 33 - protein, 23 aa, synthetic construct
  REGION 1..23: note = Description of Artificial Sequence: Synthetic peptide
DVVMTQTPLTLSVTIGQPASISC

SEQ ID NO: 34 - DNA, 45 bp, synthetic construct
  misc_feature 1..45: note = Description of Artificial Sequence: Synthetic oligonucleotide
  source 1..45: mol_type = other DNA
tggttgttacagcggccaggccagtctccaaagcgcctaatctat

SEQ ID NO: 35 - protein, 15 aa, synthetic construct
  REGION 1..15: note = Description of Artificial Sequence: Synthetic peptide
WLLQRPGQSPKRLIY

SEQ ID NO: 36 - DNA, 95 bp, synthetic construct
  misc_feature 1..95: note = Description of Artificial Sequence: Synthetic oligonucleotide
  source 1..95: mol_type = other DNA
ggagtccctgacaggttcactggcagtggatcagggacagatttcacactaaaaatcagcagagtggaggctgaggatttgggagtttattattg

SEQ ID NO: 37 - protein, 32 aa, synthetic construct
  REGION 1..32: note = Description of Artificial Sequence: Synthetic polypeptide
GVPDRFTGSGSGTDFTLKISRVEAEDLGVYYC

SEQ ID NO: 38 - DNA, 30 bp, synthetic construct
  misc_feature 1..30: note = Description of Artificial Sequence: Synthetic oligonucleotide
  source 1..30: mol_type = other DNA
ttcggtggaggcaccaagctggaaatcaaa

SEQ ID NO: 39 - protein, 10 aa, synthetic construct
  REGION 1..10: note = Description of Artificial Sequence: Synthetic peptide
FGGGTKLEIK

SEQ ID NO: 40 - protein, 7 aa, synthetic construct [sequence omitted]

SEQ ID NO: 41 - protein, 11 aa, synthetic construct
  REGION 1..11: note = Description of Artificial Sequence: Synthetic peptide
CWQGTHFPWTF